(19) United States
(12) Patent Application Publication
(10) Pub. No.: US 2010/0190813 A1
(43) Pub. Date: Jul. 29, 2010

(54) SPIRO [PIPERIDINE-4- 4' -THIENO [3,2-C] PYRAN] DERIVATIVES AND RELATED COMPOUNDS AS INHIBITORS OF THE SIGMA RECEPTOR FOR THE TREATMENT OF PSYCHOSIS

(76) Inventors: Christoph Oberdorf, Munster (DE); Dirk Schepmann, Munster (DE); Bernhard Wünsch, Munster (DE); Daniel Zamanillo-Castanedo, Barcelona (ES)

Correspondence Address: Cooper & Dunham LLP, 30 Rockefeller Plaza, 20th Floor, New York, NY, 10112, US

(21) Appl. No.: 12/452,199

(22) PCT Filed: Jun. 20, 2008
(86) PCT No.: PCT/EP2008/005011

§ 371 Date: Apr. 12, 2010

(30) Foreign Application Priority Data
Jun. 20, 2007 (EP) 07384027.4

(51) Int. Cl. A61K 31/438; C07D 491/20; A61P 1/12; A61P 3/06; A61P 25/06; A61P 3/04; A61P 19/02; A61P 1/04; A61P 9/06; A61P 9/12; A61P 25/36; A61P 25/08; A61P 25/18; A61P 29/00; A61P 37/00

(52) U.S. Cl. 514/278; 546/17

(57) ABSTRACT

The present invention relates to compounds having pharmacological activity towards the sigma (σ) receptor, and more particularly to some thieno-pyrano-pyrazole derivatives, to processes of preparation of such compounds, to pharmaceutical compositions comprising them, and to their use in therapy and prophylaxis, in particular for the treatment of psychosis or pain.

### FIELD OF THE INVENTION

The present invention relates to compounds having pharmacological activity towards the sigma (σ) receptor, and more particularly to some thieno-pyrano-pyrazole derivatives, to processes of preparation of such compounds, to pharmaceutical compositions comprising them, and to their use in therapy and prophylaxis, in particular for the treatment of psychosis.

### BACKGROUND OF THE INVENTION

The search for new therapeutic agents has been greatly aided in recent years by better understanding of the structure of proteins and other biomolecules associated with target diseases. One important class of these proteins is the sigma (σ) receptor, a cell surface receptor of the central nervous system (CNS) which may be related to the dysphoric, hallucinogenic and cardiac stimulant effects of opioids. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease (Walker, J. M. et al, Pharmacological Reviews, 1990, 42, 355). It has been reported that the known sigma receptor ligand rimcazole clinically shows effects in the treatment of psychosis (Snyder, S. H., Largent, B. L. J. Neuropsychiatry 1989, 1, 7). The sigma binding sites have preferential affinity for the dextrorotatory isomers of certain opiate benzomorphans, such as (+)SKF 10047, (+)cyclazocine, and (+)pentazocine and also for some narcoleptics such as haloperidol.

The sigma receptor has at least two subtypes, which may be discriminated by stereoselective isomers of these pharmacoactive drugs. SKF 10047 has nanomolar affinity for the sigma 1 (σ-1) site, and has micromolar affinity for the sigma (σ-2) site. Haloperidol has similar affinities for both subtypes. Endogenous sigma ligands are not known, although progesterone has been suggested to be one of them. Possible sigma-site-mediated drug effects include modulation of glutamate receptor function, neurotransmitter response, neuroprotection, behavior, and cognition (Quirion, R. et al. Trends Pharmacol. Sci., 1992, 13:85-86). Most studies have implied that sigma binding sites (receptors) are plasmalemmal elements of the signal transduction cascade. Drugs reported to be selective sigma ligands have been evaluated as antipsychotics (Hanner, M. et al. Proc. Natl. Acad. Sci., 1996, 93:8072-8077). The existence of sigma receptors in the CNS, immune and endocrine systems have suggested a likelihood that it may serve as link between the three systems.

There is still a need to find compounds that have pharmacological activity towards the sigma receptor, being both effective and selective, and having good “drugability” properties, i.e. good pharmaceutical properties related to administration, distribution, metabolism and excretion.

The closest technique known comprises benzimidazoles of WO2003035065 for the inhibition of kinases. Tetrahydro-pyranopyrazole compounds displaying cannabinoid modulating activity are disclosed in WO2007001939 and FR2875230. None of the them presents spiro-pyrano-pyrazole variants or analogues.

Spiropiperidines are known as potent ligands to sigma receptors (Maier et al, J Med Chem, 2002, 45, 438-448 and Maier et al, J Med Chem, 2002, 45, 4923-4930). However, such spiropiperidines show benzofuran and benzopyran rings.

### SUMMARY OF THE INVENTION

We have now found a family of structurally distinct thieno-pyrano-pyrazole derivatives which are particularly selective inhibitors of the sigma receptor.

The invention is directed to compounds of general formula (I) or (Ia),

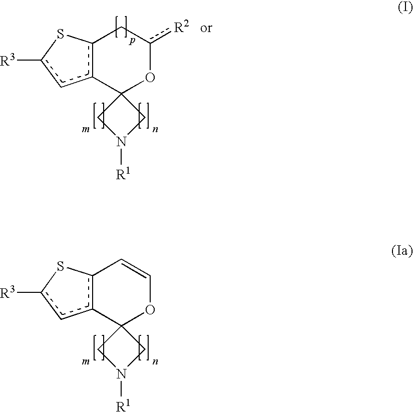

wherein
m is selected from 1, 2 or 3, n is selected from 1, 2 or 3, and m+n is either 3, 4 or 5;
p is selected from 0 or 1;
the dotted lines [symbol] are either a double or a single bond;
R¹ is selected from hydrogen; an optionally at least monosubstituted, linear or branched C₁₋₁₈-aliphatic group; an optionally at least monosubstituted aryl; an optionally at least monosubstituted heterocyclyl; an optionally at least monosubstituted cycloalkyl; an optionally at least monosubstituted alkyl-aryl; an optionally at least monosubstituted alkyl-heterocyclyl; or an optionally at least monosubstituted alkyl-cycloalkyl;
R² is selected from hydrogen; an optionally at least monosubstituted, linear or branched C₁₋₆-aliphatic group; (CH₂)_{q}CN with q being 0, 1, 2, 3 or 4; O—R with R being H or an optionally at least monosubstituted, linear or branched C₁₋₆-aliphatic group; ═O; (CH₂)ₛCOR*; (CH₂)ₛNR*R**; CONR*R**; (CH₂)ₛCO₂R*; CH═NOR*; (CH₂)ₛOR*;
   with
   R* being H; or an optionally at least monosubstituted, linear or branched C₁₋₆-aliphatic group;
   R** being an optionally at least monosubstituted, linear or branched C₁₋₆-aliphatic group;
   s being 0, 1, or 2;
R³ is selected from hydrogen; an optionally at least monosubstituted, linear or branched C₁₋₁₈-aliphatic group; NO₂; SO₃H; COR″′; (CH₂)ᵣNR′R″; CO₂R′; an optionally at least monosubstituted alkyl-aryl with alkyl optionally being substituted by OH; an optionally at least monosubstituted alkyl-heterocyclyl with alkyl optionally being substituted by OH; or an optionally at least monosubstituted alkyl-cycloalkyl with alkyl optionally being substituted by OH;
   with
   R′ being H; or an optionally at least monosubstituted, linear or branched C₁₋₆-aliphatic group;
   R″ being an optionally at least monosubstituted, linear or branched C₁₋₆-aliphatic group;
   R″′ being H; an optionally at least monosubstituted, linear or branched C₁₋₆-aliphatic group; or an optionally at least monosubstituted aryl, an optionally at least monosubstituted heterocyclyl, or an optionally at least monosubstituted cycloalkyl;
   r being 0, 1, or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment the invention is also directed to compounds of general formula (I) or (Ia),

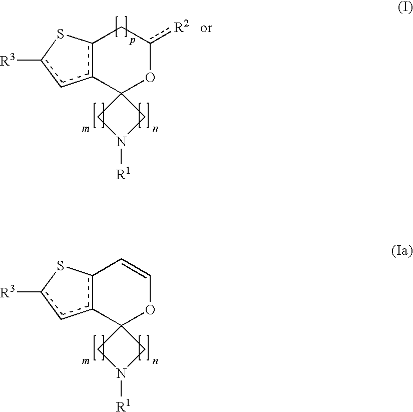

wherein
m is selected from 1, 2 or 3, n is selected from 1, 2 or 3, and m+n is either 3, 4 or 5;
p is selected from 0 or 1;
the dotted lines [symbol] are either a double or a single bond;
R¹ is selected from hydrogen; an optionally at least monosubstituted, linear or branched C₁₋₁₈-aliphatic group; an optionally at least monosubstituted aryl; an optionally at least monosubstituted heterocyclyl; an optionally at least monosubstituted cycloalkyl; an optionally at least monosubstituted alkyl-aryl; an optionally at least monosubstituted alkyl-heterocyclyl; or an optionally at least monosubstituted alkyl-cycloalkyl;
R² is selected from hydrogen; an optionally at least monosubstituted, linear or branched C₁₋₆-aliphatic group; (CH₂)_{q}CN with q being 0, 1, 2, 3 or 4; O—R with R being H or an optionally at least monosubstituted, linear or branched C₁₋₆-aliphatic group; (CH₂)ₛCOR*; (CH₂)ₛNR*R**; CONR*R**; (CH₂)ₛCO₂R*; CH═NOR*; (CH₂)ₛOR*;
   with
   R* being H; or an optionally at least monosubstituted, linear or branched C₁₋₆-aliphatic group;
   R** being an optionally at least monosubstituted, linear or branched C₁₋₆-aliphatic group; or an optionally at least monosubstituted aryl or alkyl-aryl;
   s being 0, 1, or 2;
R³ is selected from hydrogen; halogen; an optionally at least monosubstituted, linear or branched C₁₋₁₈-aliphatic group; CN; CH(═NOH); NO₂; SO₃H; COR″′; (CH₂)ᵣNR′R″; CH(OR′)(OR″); CO₂R′; an optionally at least monosubstituted alkyl-aryl with alkyl optionally being substituted by OH; an optionally at least monosubstituted alkyl-heterocyclyl with alkyl optionally being substituted by OH or forming a C(O)-keto-group; or an optionally at least monosubstituted alkyl-cycloalkyl with alkyl optionally being substituted by OH;
   with
   R′ being H; or an optionally at least monosubstituted, linear or branched C₁₋₆-aliphatic group;
   R″ being an optionally at least monosubstituted, linear or branched C₁₋₆-aliphatic group;
   R″′ being H; an optionally at least monosubstituted, linear or branched C₁₋₆-aliphatic group; or an optionally at least monosubstituted aryl, an optionally at least monosubstituted heterocyclyl, or an optionally at least monosubstituted cycloalkyl;
   r being 0, 1, or 2;
      optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof;

In one embodiment, one or both of the following provisos apply:
if R² and R³ are both hydrogen, R¹ may not be methyl; and/or
if R² and R³ are both hydrogen, R¹ may not be hydrogen.

In the context of this invention, alkyl radical or group is understood as meaning saturated and unsaturated, linear or branched hydrocarbons, which can be unsubstituted or mono- or polysubstituted. Thus unsaturated alkyl is understood to encompass alkenyl and alkinyl groups, like e.g. —CH═CH—CH₃ or —C≡C—CH₃, while saturated alkyl encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₆-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₈-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc.

In the context of this invention aliphatic group or radical includes alkyl (saturated), alkenyl (unsaturated alkyl) and alkinyl (unsaturated alkyl) and thus is synonymous for: saturated or unsaturated alkyl (see above).

In the context of this invention cycloalkyl radical or group is understood as meaning saturated and unsaturated (but not aromatic) cyclic hydrocarbons (without a heteroatom in the ring), which can be unsubstituted or mono- or polysubstituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-, C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. However, mono- or polyunsaturated, preferably monounsaturated, cycloalkyls also in particular fall under the term cycloalkyl as long as the cycloalkyl is not an aromatic system. The alkyl and cycloalkyl radicals are preferably methyl, ethyl, vinyl (ethenyl), propyl, allyl (2-propenyl), 1-propinyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, cyclopropyl, 2-methylcyclopropyl, cyclopropylmethyl, cyclobutyl, cyclopentyl, cyclopentylmethyl, cyclohexyl, cycloheptyl, cyclooctyl, and also adamantly.

In the context of this invention alkyl-cycloalkyl is understood as meaning a cycloalkyl group (see above) being connected to another atom through a C₁₋₆-alkyl group (see above), whereas the C₁₋₆-alkyl-group is always saturated and unsubstituted, and linear or branched.

In connection with alkyl or aliphatic group—unless defined otherwise—the term substituted in the context of this invention is understood as meaning replacement of at least one hydrogen radical by F, Cl, Br, I, NH₂, SH or OH, “polysubstituted” radicals being understood as meaning that the replacement takes effect both on different and on the same atoms several times with the same or different substituents, for example three times on the same C atom, as in the case of CF₃, or at different places, as in the case of e.g. —CH(OH)—CH═CH—CHCl₂.

The term (CH₂)₃₋₆ is to be understood as meaning —CH₂—CH₂—CH₂—, —CH₂—CH₂—CH₂—CH₂—, —CH₂—CH₂—CH₂—CH₂—CH₂— and —CH₂—CH₂—CH₂—CH₂—CH₂—CH₂—, (CH₂)₁₋₄ is to be understood as meaning —CH₂—, —CH₂—CH₂—, —CH₂—CH₂—CH₂— and —CH₂—CH₂—CH₂—CH₂—, (CH₂)₄₋₅ is to be understood as meaning —CH₂—CH₂—CH₂—CH₂— and —CH₂—CH₂—CH₂—CH₂—CH₂—, etc.

An aryl radical or group is understood as meaning ring systems with at least one aromatic ring but without heteroatoms even in only one of the rings. Examples are phenyl, naphthyl, fluoranthenyl, fluorenyl, tetralinyl or indanyl, in particular 9H-fluorenyl or anthracenyl radicals, which can be unsubstituted or monosubstituted or polysubstituted.

In the context of this invention alkyl-aryl is understood as meaning an aryl group (see above) being connected to another atom through a C₁₋₆-alkyl-group (see above), whereas the C₁₋₆-alkyl-group is always saturated and unsubstituted, and linear or branched.

A heterocyclyl radical or group is understood as meaning heterocyclic ring systems, saturated or unsaturated ring which contains one or more heteroatoms from the group consisting of nitrogen, oxygen and/or sulfur in the ring and can also be mono- or polysubstituted. Examples which may be mentioned from the group of heteroaryls are furan, benzofuran, thiophene, benzothiophene, pyrrole, pyridine, pyrimidine, pyrazine, quinoline, isoquinoline, phthalazine, benzo-1,2,5-thiadiazole, benzothiazole, indole, benzotriazole, benzodioxolane, benzodioxane, carbazole and quinazoline.

In the context of this invention alkyl-heterocycyl is understood as meaning a heterocyclyl group (see above) being connected to another atom through a C₁₋₆-alkyl group (see above), whereas the C₁₋₆-alkyl-group is always saturated and unsubstituted, and linear or branched.

In connection with aryl or alkyl-aryl, cycloalkyl or alkyl-cycloalkyl, heterocyclyl or alkyl-heterocyclyl, substituted is understood—unless defined otherwise—as meaning substitution of the ring-system of the aryl or alkyl-aryl, cycloalkyl or alkyl-cycloalkyl; heterocyclyl or alkyl-heterocyclyl by OH, SH, ═O, halogen (F, Cl, Br, I), CN, NO₂, COOH; NRₓR_{y}, with Rₓ and R_{y} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆-alkyl-group; a saturated or unsaturated, linear or branched, substituted or unsubstituted —C(O)—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes which are complexed via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH4, but in particular (mono)- or (di)sodium, (mono)- or (di)potassium, magnesium or calcium salts.

These physiologically acceptable salts can also be formed with anions or acids in the context of this invention is understood as meaning salts of at least one of the compounds used according to the invention—usually protonated, for example on the nitrogen—as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be in crystalline form or either as free compounds or as solvates and it is intended that those forms are within the scope of the present invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent) especially including hydrates and alcoholates, e.g. methanolate.

Any compound that is a prodrug of a compound of formula (I) is within the scope of the invention. The term “prodrug” is used in its broadest sense and encompasses those Derivatives that are converted in vivo to the compounds of the invention. Such Derivatives would readily occur to those skilled in the art, and include, depending on the functional groups present in the molecule and without limitation, the following Derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or ¹⁵N-enriched nitrogen are within the scope of this invention.

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I) or, or of its salts, solvates or prodrugs.

Another preferred embodiment of the invention refers to a compound of general formula Ic,

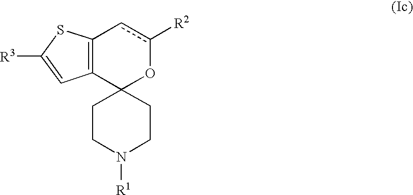

wherein
m is selected from 1, 2 or 3, n is selected from 1, 2 or 3, and m+n is either 3, 4 or 5;
p is selected from 0 or 1;
the dotted lines [symbol] are either a double or a single bond;
R¹ is selected from hydrogen; an optionally at least monosubstituted, linear or branched C₁₋₁₈-aliphatic group; an optionally at least monosubstituted aryl; an optionally at least monosubstituted heterocyclyl; an optionally at least monosubstituted cycloalkyl; an optionally at least monosubstituted alkyl-aryl; an optionally at least monosubstituted alkyl-heterocyclyl; or an optionally at least monosubstituted alkyl-cycloalkyl;
R² is selected from hydrogen; an optionally at least monosubstituted, linear or branched C₁₋₆-aliphatic group; (CH₂)_{q}CN with q being 0, 1, 2, 3 or 4; O—R with R being H or an optionally at least monosubstituted, linear or branched C₁₋₆-aliphatic group; (CH₂)ₛCOR*; (CH₂)ₛNR*R**; CONR*R**; (CH₂)ₛCO₂R*; CH═NOR*; (CH₂)ₛOR*;
   with
   R* being H; or an optionally at least monosubstituted, linear or branched C₁₋₆-aliphatic group;
   R** being an optionally at least monosubstituted, linear or branched C₁₋₆-aliphatic group; or an optionally at least monosubstituted aryl or alkyl-aryl;
   s being 0, 1, or 2;
R³ is selected from hydrogen; halogen; an optionally at least monosubstituted, linear or branched C₁₋₁₈-aliphatic group; CN; CH(═NOH); NO₂; SO₃H; COR″′; (CH₂)ᵣNR′R″; CH(OR′)(OR″); CO₂R′; an optionally at least monosubstituted alkyl-aryl with alkyl optionally being substituted by OH; an optionally at least monosubstituted alkyl-heterocyclyl with alkyl optionally being substituted by OH or forming a C(O)-keto-group; or an optionally at least monosubstituted alkyl-cycloalkyl with alkyl optionally being substituted by OH;
   with
   R′ being H; or an optionally at least monosubstituted, linear or branched C₁₋₆-aliphatic group;
   R″ being an optionally at least monosubstituted, linear or branched C₁₋₆-aliphatic group;
   R″′ being H; an optionally at least monosubstituted, linear or branched C₁₋₆-aliphatic group; or an optionally at least monosubstituted aryl, an optionally at least monosubstituted heterocyclyl, or an optionally at least monosubstituted cycloalkyl;
   r being 0, 1, or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In one embodiment, one or both of the following provisos apply:
if R² and R³ are both hydrogen, R¹ may not be methyl; and/or
if R² and R³ are both hydrogen, R¹ may not be hydrogen.

In a preferred embodiment of the compound according to the invention according to general formula (I), (Ia) or (Ic) R¹ is selected from H; an optionally at least monosubstituted, linear or branched C₁₋₁₈-aliphatic group; an optionally at least monosubstituted alkyl-aryl; an optionally at least monosubstituted alkyl-heterocyclyl; or an optionally at least monosubstituted alkyl-cycloalkyl.

In another preferred embodiment of the compound according to the invention according to general formula (I) or (Ia) R² is selected from H; (CH₂)_{q}CN with q being 0, 1, 2, 3 or 4; or OR with R being H or an optionally at least monosubstituted, linear or branched C₁₋₆-aliphatic group; preferably R² is selected from H; CN; CH₂CN; or OR with R being H or a linear or branched C₁₋₄-alkyl group, more preferably R² is selected from H, CN; CH₂CN; OH or OCH₃.

In another preferred embodiment of the compound according to the invention according to general formula (I), (Ia) or (Ic) R² is selected from hydrogen; an optionally at least monosubstituted, linear or branched C₁₋₆-aliphatic group; or OR with R being H or an optionally at least monosubstituted, linear or branched C₁₋₆-aliphatic group; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O)—O—R²², with q being 0, or 1 and R²² being H; or an optionally at least monosubstituted, linear or branched C₁₋₆-aliphatic group.

In another preferred embodiment of the compound according to the invention according to general formula (I) or (Ia) R³ is selected from H; or an optionally at least monosubstituted, linear or branched C₁₋₆-aliphatic group; especially R³ is selected from H; or an optionally at least monosubstituted, linear or branched C₁₋₆-alkyl group.

In another preferred embodiment of the compound according to the invention according to general formula (I), (Ia) or (Ic) R³ is selected from hydrogen; halogen; CN; CH(═NOH); an optionally at least monosubstituted, linear or branched C₁₋₁₀-aliphatic group; CH₂—R³³; C(O)—R³³; C(O)O—R³³; CHOH—R³³; CH₂N(R³³)₂ with R³³ being H; or an optionally at least monosubstituted, linear or branched C₁₋₆-aliphatic group; or an optionally at least monosubstituted aryl.

In another preferred embodiment of the compound according to the invention according to general formula (I) or (Ia) m and n are selected from 1 or 2 and m+n are selected from 3 or 4.

In another very preferred embodiment of the compound according to the invention the compounds are compounds according to general formula (Id)

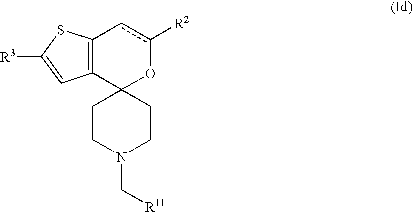

wherein
the bond with the dotted line [symbol] either is a double or a single bond;
R¹¹ is selected from an optionally at least monosubstituted, linear or branched C₁₋₉-aliphatic group; an optionally at least monosubstituted aryl or alkyl-aryl; an optionally at least monosubstituted heterocyclyl or alkyl-heterocyclyl; an optionally at least monosubstituted cycloalkyl or alkyl-cycloalkyl;
R² is selected from hydrogen; an optionally at least monosubstituted, linear or branched C₁₋₆-aliphatic group; or OR with R being H or an optionally at least monosubstituted, linear or branched C₁₋₆-aliphatic group; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O)—O—R²², with q being 0, or 1 and R²² being H; or an optionally at least monosubstituted, linear or branched C₁₋₆-aliphatic group;
R³ is selected from hydrogen; halogen; CN; CH(═NOH); an optionally at least monosubstituted, linear or branched C₁₋₁₀-aliphatic group; CH₂—R³³; C(O)—R³³; C(O)O—R³³; CHOH—R³³; CH₂N(R³³)₂ with R³³ being H; an optionally at least monosubstituted, linear or branched C₁₋₆-aliphatic group; or an optionally at least monosubstituted aryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compounds according to general formula (Id) R¹¹ is selected from an optionally at least monosubstituted, linear or branched C₁₋₉-aliphatic group; an optionally at least monosubstituted aryl or alkyl-aryl; an optionally at least monosubstituted heterocyclyl; or an optionally at least monosubstituted cycloalkyl; preferably is phenyl, p-methoy-phenyl, cyclohexyl, thiophene, benzyl; branched or linear C₃₋₇-alkyl; or branched or linear C₃₋₇-alkenyl.
In a preferred embodiment of the compounds according to general formula (Id) R² is selected from hydrogen; an unsubstituted or by at least one of OH, F, or Cl substituted, linear or branched C₁₋₆-aliphatic group; or OR with R being H or CH3; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O)—O—R²², with q being 0, or 1 and R²² being H, CH₃, or C₂H₅.

In a preferred embodiment of the compounds according to general formula (Id) R³ is selected from hydrogen; halogen; CN; CH(═NOH); a linear or branched, unsubstituted or by at least one of OH, Cl, or F substituted C₁₋₆-aliphatic group; CH₂—R³³; C(O)—R³³; C(O)O—R³³; CHOH—R³³; CH₂N(R³³)₂ with R³³ being H; CH₃; C₂H₅; or phenyl.

In another very preferred embodiment of the compound according to the invention the compounds are compounds according to general formula (Ib)

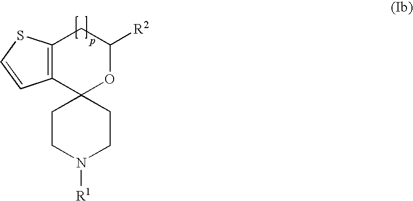

wherein
p is selected from 0 or 1;
R¹ is selected from hydrogen; an optionally at least monosubstituted, linear or branched C₁₋₁₈-aliphatic group; an optionally at least monosubstituted aryl; an optionally at least monosubstituted heterocyclyl; an optionally at least monosubstituted cycloalkyl; an optionally at least monosubstituted alkyl-aryl; an optionally at least monosubstituted alkyl-heterocyclyl; an optionally at least monosubstituted alkyl-cycloalkyl; or COOR′ with R′ being either H or C₁₋₄-alkyl;
R² is selected from hydrogen; an optionally at least monosubstituted, linear or branched C₁₋₆-aliphatic group; (CH₂)_{q}CN with q being 0, or 1; or OR with R being H or an optionally at least monosubstituted, linear or branched C₁₋₆-aliphatic group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention according to general formula (Ib) R² is selected from H; CH₂CN; CN; or OR with R being H or an optionally at least monosubstituted, linear or branched C₁₋₆-alkyl group; especially R² is selected from H; CH₂CN; CN; OH or a linear or branched OC₁₋₄-alkyl group; more especially R² is selected from H, CN, CH₂CN, OH or OCH₃.

In another preferred embodiment of the compound according to the invention according to general formula (Ib) R¹ is selected from hydrogen; an optionally at least monosubstituted, linear or branched C₁₋₁₈-aliphatic group; an optionally at least monosubstituted alkyl-aryl; an optionally at least monosubstituted alkyl-heterocyclyl; or an optionally at least monosubstituted alkyl-cycloalkyl.

In another preferred embodiment of the compound according to the invention according to general formula (Ib) R¹ is selected from
hydrogen, C₁₋₁₀-alkyl, linear or branched; C₁₋₁₀-alkenyl, linear or branched; optionally at least mono-substituted C₁₋₆-alkyl-aryl; optionally at least mono-substituted C₁₋₆-alkyl-heterocyclyl; or optionally at least mono-substituted C₁₋₆-alkyl-cycloalkyl;
preferably R¹ is selected from hydrogen; C₁₋₁₀-alkyl, linear or branched; C₂₋₈-alkenyl, linear or branched; optionally at least mono-substituted C₁₋₆-alkyl-aryl; optionally at least mono-substituted C₁₋₆-alkyl-heterocyclyl; or optionally at least mono-substituted C₁₋₆-alkyl-C₄₋₈-cycloalkyl.

In another highly preferred embodiment of the compound according to the invention the compounds are compounds according to general formula (Ib)

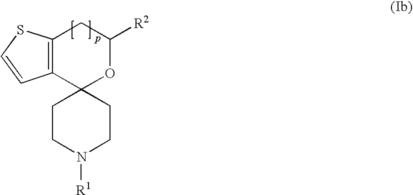

wherein
p is selected from 0 or 1;
R¹ is selected from hydrogen; C₁₋₁₀-alkyl, linear or branched; C₂₋₈-alkenyl, linear or branched; optionally at least mono-substituted C₁₋₆-alkyl-aryl; optionally at least mono-substituted C₁₋₆-alkyl-heterocyclyl; or optionally at least mono-substituted C₁₋₆-alkyl-C₄₋₈-cycloalkyl;
R² is selected from H; CN; CH₂CN; OH or a linear or branched OC₁₋₄-alkyl group;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention the compounds according to general formula (I) or (Ia) are selected from:
Ethyl-6′-methoxy-6′,7′-dihydrospiro[piperidin-4,4′-thieno[3,2-c]pyran]-1-carboxylate;
6′-Methoxy-6′,7′-dihydrospiro[piperidin-4,4′-thieno[3,2-c]pyran;
1-Benzyl-6′-methoxy-6′,7′-dihydrospiro[piperidin-4,4′-thieno[3.2-c]pyran];
6′-Methoxy-1-(2-phenylethyl)-6′,7′-dihydrospiro[piperidin-4,4′-thieno[3.2-c]pyran];
1-(Cyclohexylmethyl)-6′-Methoxy-6′,7′-dihydrospiro[piperidin-4,4′-thieno[3.2-c]pyran];
6′-Methoxy-1-(3-methylbutyl)-6′,7′-dihydrospiro[piperidin-4,4′-thieno[3.2-c]pyran];
6′-Methoxy-1-(3-methylbut-2-enyl)-6′,7′-dihydrospiro[piperidin-4,4′-thieno[3.2-c]pyran];
1-Butyl-6′-methoxy-6′,7′-dihydrospiro[piperidin-4,4′-thieno[3.2-c]pyran];
6′-Methoxy-1-pentyl-6′,7′-dihydrospiro[piperidin-4,4′-thieno[3.2-c]pyran];
6′-Methoxy-1-octyl-6′,7′-dihydrospiro[piperidin-4,4′-thieno[3.2-c]pyran];
3-Methoxy-1-(3-phenylpropyl)-6′,7′-dihydrospiro[piperidin-4,4′-thieno[3.2-c]pyran];
3′-Methoxy-1-(4-phenylbutyl)-6′,7′-dihydrospiro[piperidin-4,4′-thieno[3.2-c]pyran];
1-(4-Fluorbenzyl)-6′-Methoxy-6′,7′-dihydrospiro[piperidin-4,4′-thieno[3.2-c]pyran];
6′-Methoxy-1-(4-methoxybenzyl)-6′,7′-dihydrospiro[piperidin-4,4′-thieno[3.2-c]pyran];
6′-Methoxy-1-(thien-2-ylmethyl)-6′,7′-dihydrospiro[piperidin-4,4′-thieno[3.2-c]pyran];
1-Benzylspiro[piperidin-4,4′-thieno[3,2-c]pyran];
1-Benzyl-6′,7′-dihydrospiro[piperidin-4,4′-thieno[3,2-c]pyran]-6′-carbonitrile;
{1-Benzyl-6′,7′-dihydrospiro[piperidin-4,4′-thieno[3,2-c]pyran]-6′-yl}acetonitrile;
1-Benzyl-6′,7′-dihydrospiro[piperidin-4,4′-thieno[3,2-c]pyran]; or
1-Benzyl-6′,7′-dihydrospiro[piperidin-4,4′-thieno[3,2-c]pyran]-6′-ol;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof;
   preferably from
1-Benzyl-6′-methoxy-6′,7′-dihydrospiro[piperidin-4,4′-thieno[3.2-c]pyran];
6′-Methoxy-1-(2-phenylethyl)-6′,7′-dihydrospiro[piperidin-4,4′-thieno[3.2-c]pyran];
1-(Cyclohexylmethyl)-6′-Methoxy-6′,7′-dihydrospiro[piperidin-4,4′-thieno[3.2-c]pyran];
6′-Methoxy-1-(3-methylbutyl)-6′,7′-dihydrospiro[piperidin-4,4′-thieno[3.2-c]pyran];
6′-Methoxy-1-(3-methylbut-2-enyl)-6′,7′-dihydrospiro[piperidin-4,4′-thieno[3.2-c]pyran];
1-Butyl-6′-methoxy-6′,7′-dihydrospiro[piperidin-4,4′-thieno[3.2-c]pyran];
6′-Methoxy-1-pentyl-6′,7′-dihydrospiro[piperidin-4,4′-thieno[3.2-c]pyran];
6′-Methoxy-1-octyl-6′,7′-dihydrospiro[piperidin-4,4′-thieno[3.2-c]pyran];
3′-Methoxy-1-(3-phenylpropyl)-6′,7′-dihydrospiro[piperidin-4,4′-thieno[3.2-c]pyran];
3′-Methoxy-1-(4-phenylbutyl)-6′,7′-dihydrospiro[piperidin-4,4′-thieno[3.2-c]pyran];
1-(4-Fluorbenzyl)-6′-Methoxy-6′,7′-dihydrospiro[piperidin-4,4′-thieno[3.2-c]pyran];
6′-Methoxy-1-(4-methoxybenzyl)-6′,7′-dihydrospiro[piperidin-4,4′-thieno[3.2-c]pyran];
6′-Methoxy-1-(thien-2-ylmethyl)-6′,7′-dihydrospiro[piperidin-4,4′-thieno[3.2-c]pyran];
1-Benzyl-6′,7′-dihydrospiro[piperidin-4,4′-thieno[3,2-c]pyran]-6′-carbonitrile;
{1-Benzyl-6′,7′-dihydrospiro[piperidin-4,4′-thieno[3,2-c]pyran]-6′-yl}acetonitrile;
1-Benzyl-6′,7′-dihydrospiro[piperidin-4,4′-thieno[3,2-c]pyran]; or
1-Benzyl-6′,7′-dihydrospiro[piperidin-4,4′-thieno[3,2-c]pyran]-6′-ol;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the compound according to the invention the compounds according to general formula (I), (Ia), (Ic) or (Id) are selected from:
6′-Methoxy-6′,7′-dihydrospiro[piperidin-4,4′-thieno[3,2-c]pyran;
1-Benzyl-6′-methoxy-6′,7′-dihydrospiro[piperidin-4,4′-thieno[3.2-c]pyran];
6′-Methoxy-1-(2-phenylethyl)-6′,7′-dihydrospiro[piperidin-4,4′-thieno[3.2-c]pyran];
1-(Cyclohexylmethyl)-6′-Methoxy-6′,7′-dihydrospiro[piperidin-4,4′-thieno[3.2-c]pyran];
6′-Methoxy-1-(3-methylbutyl)-6′,7′-dihydrospiro[piperidin-4,4′-thieno[3.2-c]pyran];
6′-Methoxy-1-(3-methylbut-2-enyl)-6′,7′-dihydrospiro[piperidin-4,4′-thieno[3.2-c]pyran];
1-Butyl-6′-methoxy-6′,7′-dihydrospiro[piperidin-4,4′-thieno[3.2-c]pyran];
6′-Methoxy-1-pentyl-6′,7′-dihydrospiro[piperidin-4,4′-thieno[3.2-c]pyran];
6′-Methoxy-1-octyl-6′,7′-dihydrospiro[piperidin-4,4′-thieno[3.2-c]pyran];
3′-Methoxy-1-(3-phenylpropyl)-6′,7′-dihydrospiro[piperidin-4,4′-thieno[3.2-c]pyran];
3′-Methoxy-1-(4-phenylbutyl)-6′,7′-dihydrospiro[piperidin-4,4′-thieno[3.2-c]pyran];
1-(4-Fluorbenzyl)-6′-Methoxy-6′,7′-dihydrospiro[piperidin-4,4′-thieno[3.2-c]pyran];
6′-Methoxy-1-(4-methoxybenzyl)-6′,7′-dihydrospiro[piperidin-4,4′-thieno[3.2-c]pyran];
6′-Methoxy-1-(thien-2-ylmethyl)-6′,7′-dihydrospiro[piperidin-4,4′-thieno[3.2-c]pyran];
1-Benzylspiro[piperidin-4,4′-thieno[3,2-c]pyran];
1-Benzyl-6′,7′-dihydrospiro[piperidin-4,4′-thieno[3,2-c]pyran]-6′-carbonitril;
{1-Benzyl-6′,7′-dihydrospiro[piperidin-4,4′-thieno[3,2-c]pyran]-6′-yl}acetonitril;
1-Benzyl-6′,7′-dihydrospiro[piperidin-4,4′-thieno[3,2-c]pyran];
1-Benzyl-6′,7′-dihydrospiro[piperidin-4,4′-thieno[3,2-c]pyran]-6′-ol
1-(Cyclohexylmethyl)spiro[piperidine-4,4′-thieno[3,2-c]pyrano]
1-(Cyclohexylmethyl)-6′,7′-dihydrospiro[piperidine-4,4′-thieno[3,2-c]pyrano]-6′-carbonitrile
1-(1-Benzyl-6′,7′-dihydrospiro[piperidine-4,4′-thieno[3,2-c]pyrano]-6′-yl)acetone
Methyl-2-(1-benzyl-6′,7′-dihydrospiro[piperidin-4,4′-thieno[3,2-c]pyrano]-6′-yl)-acetate
Ethyl-2-(1-benzyl-6′,7′-dihydrospiro[piperidine-4,4′-thieno[3,2-c]pyrano]-6′-yl)acetate
1-(Cyclohexylmethyl)-6′,7′-dihydrospiro[piperidine-4,4′-thieno[3,2-c]pyrano]
2-(1-Benzyl-6′,7′-dihydrospiro[piperidine-4,4′-thieno[3,2-c]pyrano]-6′-yl)ethanol
1-Benzyl-6′,7′-dihydrospiro[piperidine-4,4′-thieno[3,2-c]pyrano]-6′-carboxamide
1-Benzyl-6′,7′-dihydrospiro[piperidine-4,4′-thieno[3,2-c]pyrano]-6′-carbaldehyde
(1-Benzyl-6′,7′-dihydrospiro[piperidine-4,4′-thieno[3,2-c]pyrano]-6′-yl)methanamine
Methyl-1-benzyl-6′,7′-dihydrospiro[piperidine-4,4′-thieno[3,2-c]pyrano]-6′-carboxylate
Ethyl-1-benzyl-6′,7′-dihydrospiro[piperidine-4,4′-thieno[3,2-c]pyrano]-6′-carboxylate
(1-Benzyl-6′,7′-dihydrospiro[piperidine-4,4′-thieno[3,2-c]pyrano]-6′-yl)methanol
(1-Benzyl-6′,7′-dihydrospiro[piperidine-4,4′-thieno[3,2-c]pyrano]-6′-yl)-N,N-dimethylmethanamine
(1-Benzyl-6′,7′-dihydrospiro[piperidine-4,4′-thieno[3,2-c]pyrano]-6′-yl)-N-benzyl-methanamine
(1-Benzyl-6′-methoxy-6′,7′-dihydrospiro[piperidine-4,4′-thieno[3.2-c]pyrano]-2-carbaldehyde-dimethylacetal
1-Benzyl-2′-chloro-6′-methoxy-6′,7′-dihydrospiro[piperidine-4,4′-thieno[3.2-c]-pyrano]
1,2′-Dibenzyl-6′-methoxy-6′,7′-dihydrospiro[piperidine-4,4′-thieno[3.2-c]pyrano]
(1-Benzyl-6′-methoxy-6′,7′-dihydrospiro[piperidine-4,4′-thieno[3.2-c]pyrano]-2-carbaldehyde
(1-Benzyl-6′-methoxy-6′,7′-dihydrospiro[piperidine-4,4′-thieno[3.2-c]pyrano]-2′-yl)-methanol
(1-Benzyl-6′-methoxy-6′,7′-dihydrospiro[piperidine-4,4′-thieno[3.2-c]pyrano]-2′-yl)-N,N-dimethylmethanamine
(E/Z)-1-Benzyl-6′-methoxy-6′,7′-dihydrospiro[piperidine-4,4′-thieno[3,2-c]pyrano]-2-carbaldehydroxim
1-Benzyl-6′-methoxy-6′,7′-dihydrospiro[piperidine-4,4′-thieno[3,2-c]pyrano]-2-carbonitrile
Methyl-1-benzyl-6′-methoxy-6′,7′-dihydrospiro[piperidine-4,4′-thieno[3,2-c]pyrano]-2-carboxylate
1-(1-Benzyl-6′-methoxy-6′,7′-dihydrospiro[piperidine-4,4′-thieno[3.2-c]pyrano]-2-yl)-1-phenylmethanol
1-(1-Benzyl-6′-methoxy-6′,7′-dihydrospiro[piperidine-4,4′-thieno[3.2-c]pyrano]-2-yl)-1-phenylmethanone;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

The term “pharmacological tool” refers to the property of compounds of the invention through which they are particularly selective ligands for Sigma receptors which implies that compound of formula (I), described in this invention, can be used as a model for testing other compounds as sigma ligands, ex. a radiactive ligands being replaced, and can also be used for modeling physiological actions related to sigma receptors.

The compounds of the present invention represented by the above described formula (I) may include enantiomers depending on the presence of chiral centres or isomers depending on the presence of multiple bonds (e.g. Z, E). The single isomers, enantiomers or diastereoisomers and mixtures thereof fall within the scope of the present invention.

In general the processes are described below in the experimental part. The starting materials are commercially available or can be prepared by conventional methods.

A preferred aspect of the invention is also a process for the production of a compound according to the invention according to general formula (I), wherein a compound of formula (III)

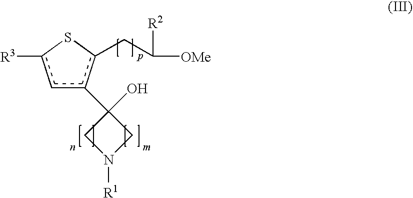

, wherein R¹, R², R³, m, n and p are as defined above, is reacted with p-toluol sulphonic acid to form a compound according the formula I. It is preferred if in this process
   R² is OCH₃; and/or
   R³ is H and/or
   p is H.

A preferred aspect of the invention is also a process for the production of a compound according to the invention according to general formula (Ib), wherein a compound of formula (IIIb)

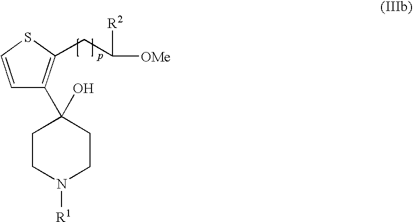

, wherein R¹, R² and p are as defined above is reacted with p-toluol sulphonic acid to form a compound according the formula (Ib). It is preferred if
R² is OCH₃ and/or
p is 1.

It is further preferred if in the process above as a next step a compound according to formula (I) or (Ib) in which R³ is C(O)OR′ is reacted with a alkaline solution in water to form a compound according to formulas (I) or (Ib) with R¹ being H.

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form is the crystalline form, including such form in pharmaceutical composition. In the case of salts and solvates the additional ionic and solvent moieties must also be non-toxic. The compounds of the invention may present different polymorphic forms, it is intended that the invention encompasses all such forms.

Another aspect of the invention refers to a pharmaceutical composition which comprises a compound according to the invention or a pharmaceutically acceptable salt, prodrug, isomer or solvate thereof, and a pharmaceutically acceptable carrier, adjuvant or vehicle. The present invention thus provides pharmaceutical compositions comprising a compound of this invention, or a pharmaceutically acceptable salt, derivative, prodrug or stereoisomers thereof together with a pharmaceutically acceptable carrier, adjuvant, or vehicle, for administration to a patient. This aspect also refers to a pharmaceutical composition which comprises a compound according to the invention or a pharmaceutically acceptable salt or solvate thereof, and a pharmaceutically acceptable carrier, adjuvant or vehicle. The present invention thus also provides pharmaceutical compositions comprising a compound of this invention, or a pharmaceutically acceptable salt thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound according to the invention in the manufacture of a medicament.

Another aspect of the invention refers to the use of a compound according to the invention in the manufacture of a medicament for the treatment or prophylaxis of a sigma receptor mediated disease or condition. A parallel aspect of the invention refers to the use of a compound according to the invention for the treatment or prophylaxis of a sigma receptor mediated disease or condition. A preferred embodiment of this is this use wherein the disease is diarrhoea, lipoprotein disorders, metabolic syndrome, treatment of elevated triglyceride levels, chylomicronemia, hyperlipoproteinemia; hyperlipidemia, especially mixed hyperlipidemia; hypercholesterolemia, dysbetalipoproteinemia, hypertriglyceridemia including both the sporadic and familial disorder (inherited hypertriglyceridemia), migraine, obesity, arthritis, hypertension, arrhythmia, ulcer, learning, memory and attention deficits, cognition disorders, neurodegenerative diseases, demyelinating diseases, addiction to drugs and chemical substances including cocaine, amphetamine, ethanol and nicotine, tardive diskinesia, ischemic stroke, epilepsy, stroke, depression, stress, psychotic condition, schizophrenia; inflammation, autoimmune diseases or cancer.

A preferred embodiment of this is this use wherein the disease is pain, especially neuropathic pain, inflammatory pain or other pain conditions, allodynia and/or hyperalgesia, especially mechanical allodynia.

Another aspect of the invention refers to the use of a compound according to the invention as pharmacological tool or as anxiolytic or immunosuppressant.

The term “pharmacological tool” refers to the property of compounds of the invention through which they are particularly selective ligands for Sigma receptors which implies that compound of formula I, described in this invention, can be used as a model for testing other compounds as Sigma ligands, ex. a radiactive ligands being replaced, and can also be used for modeling physiological actions related to Sigma receptors.

Another aspect of this invention relates to a method of treating or preventing a sigma receptor mediated disease which method comprises administering to a patient in need of such a treatment a therapeutically effective amount of a compound as above defined or a pharmaceutical composition thereof. Among the sigma mediated diseases that can be treated are diarrhoea, lipoprotein disorders, metabolic syndrome, treatment of elevated triglyceride levels, chylomicronemia, hyperlipoproteinemia; hyperlipidemia, especially mixed hyperlipidemia; hypercholesterolemia, dysbetalipoproteinemia, hypertriglyceridemia including both the sporadic and familial disorder (inherited hypertriglyceridemia), migraine, obesity, arthritis, hypertension, arrhythmia, ulcer, learning, memory and attention deficits, cognition disorders, neurodegenerative diseases, demyelinating diseases, addiction to drugs and chemical substances including cocaine, amphetamine, ethanol and nicotine, tardive diskinesia, ischemic stroke, epilepsy, stroke, depression, stress, pain, especially neuropathic pain, inflammatory pain or other pain conditions, allodynia and/or hyperalgesia, especially mechanical allodynia, psychotic condition, schizophrenia; inflammation, autoimmune diseases or cancer; disorders of food ingestion, the regulation of appetite, for the reduction, increase or maintenance of body weight, for the prophylaxis and/or treatment of obesity, bulimia, anorexia, cachexia or type II diabetes, preferably type II diabetes caused by obesity. The compounds of the invention can also be employed as pharmacological tool or as anxiolytic or immunosuppressant.

The compounds of the invention may be synthesized following one of the Reaction Schemes A, B, C, D, E or F set out below:

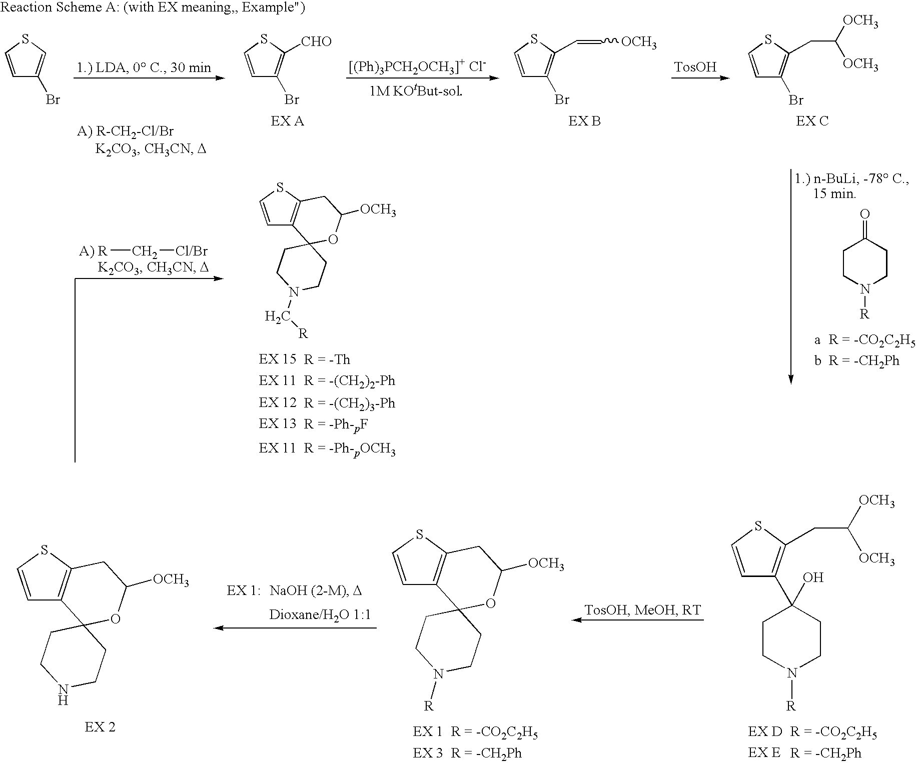

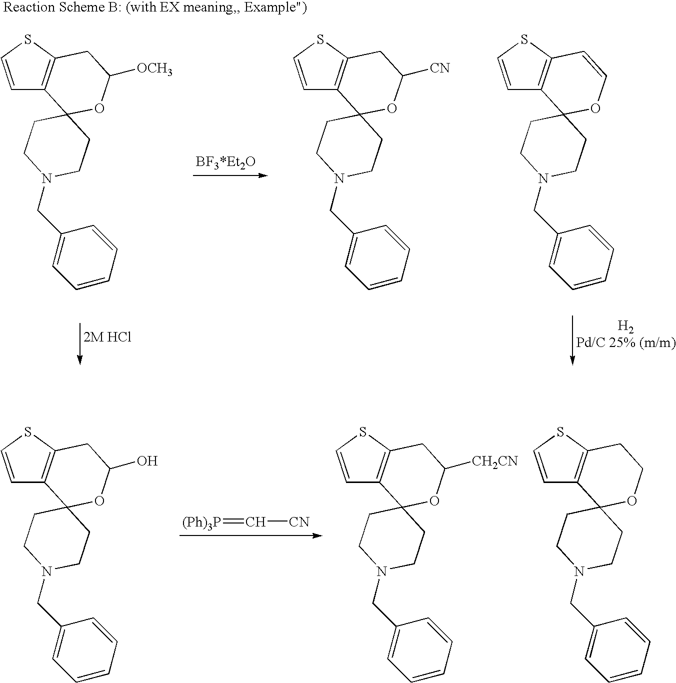

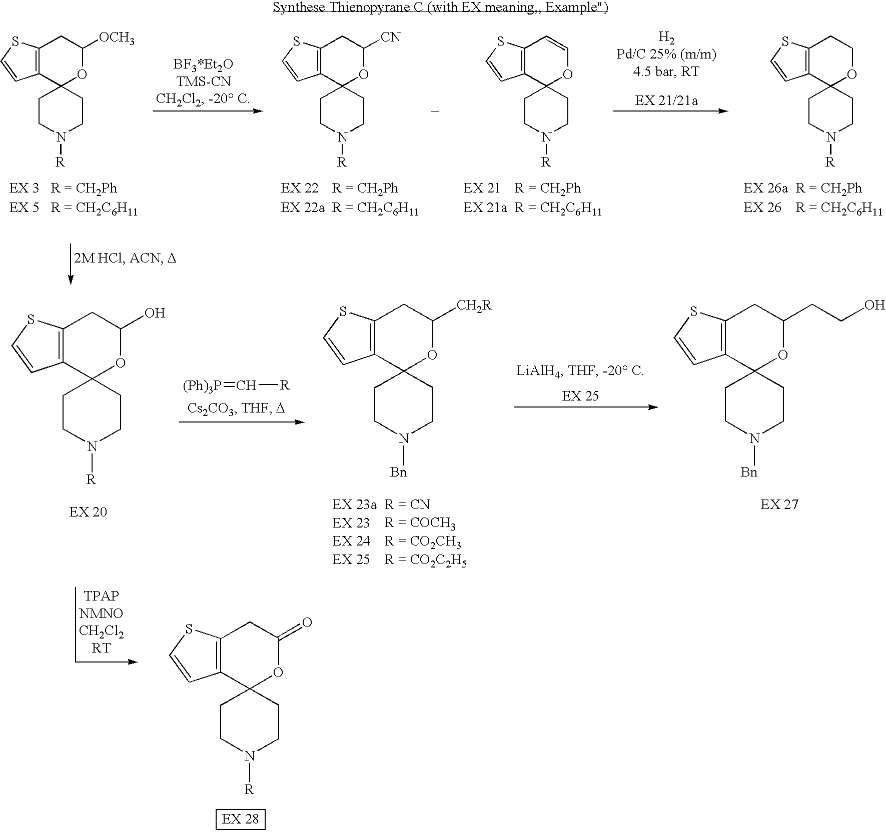

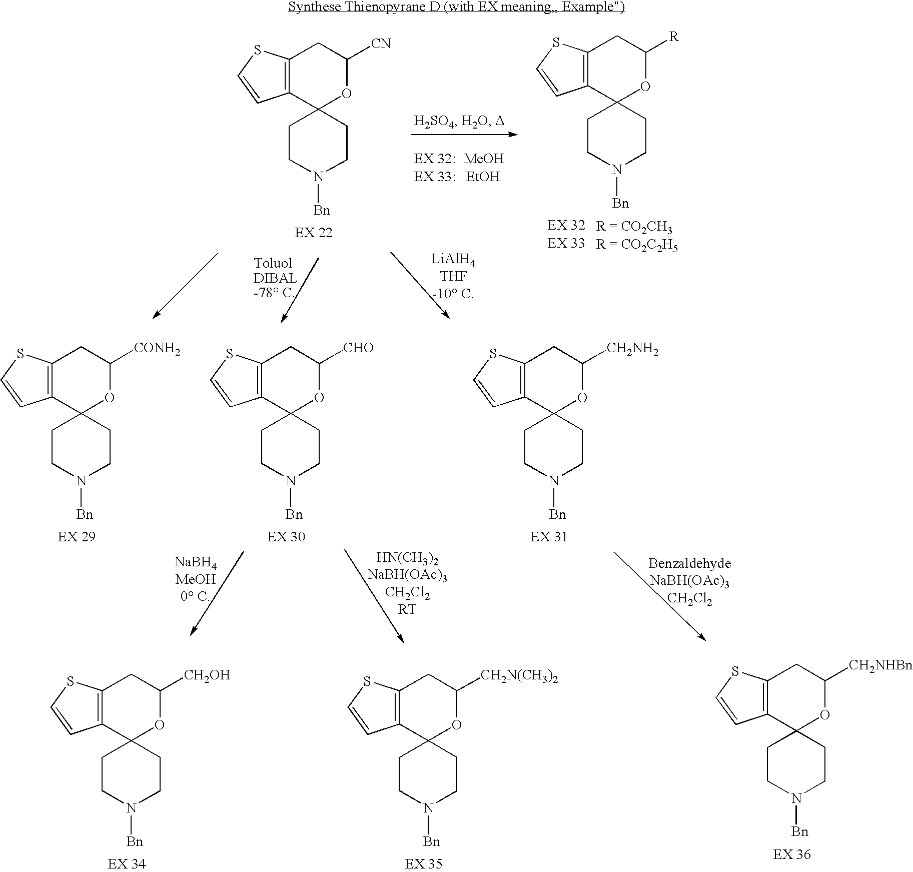

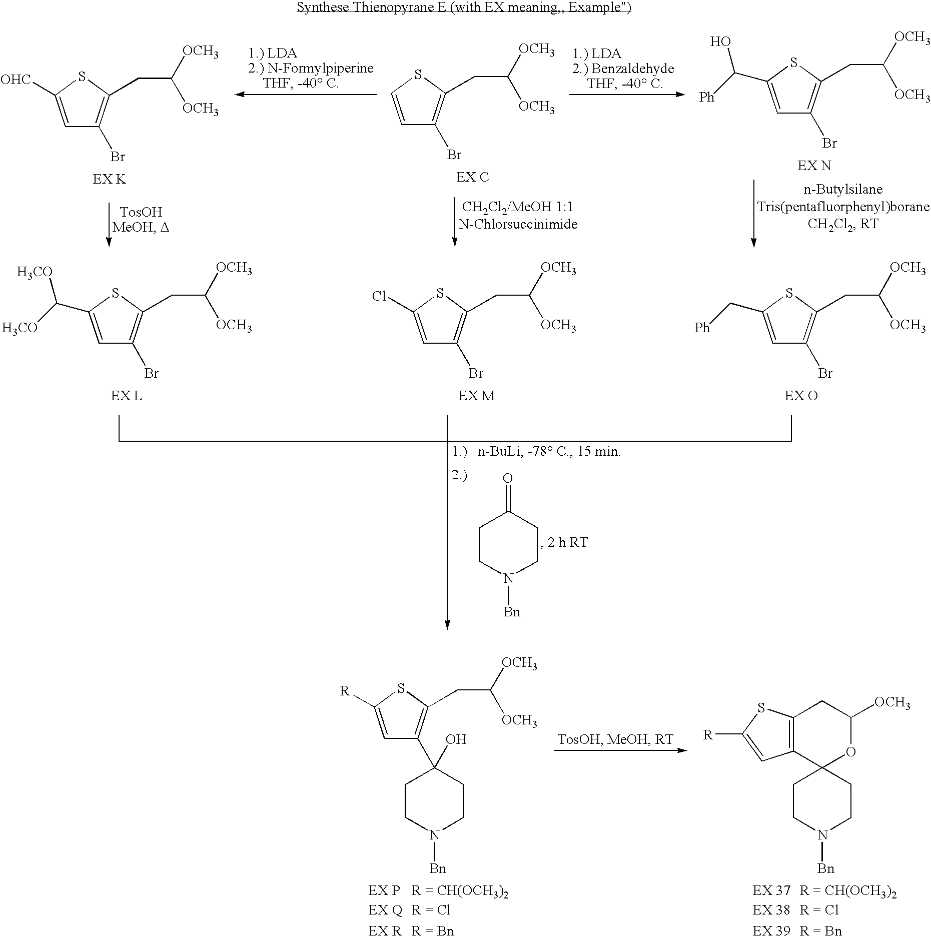

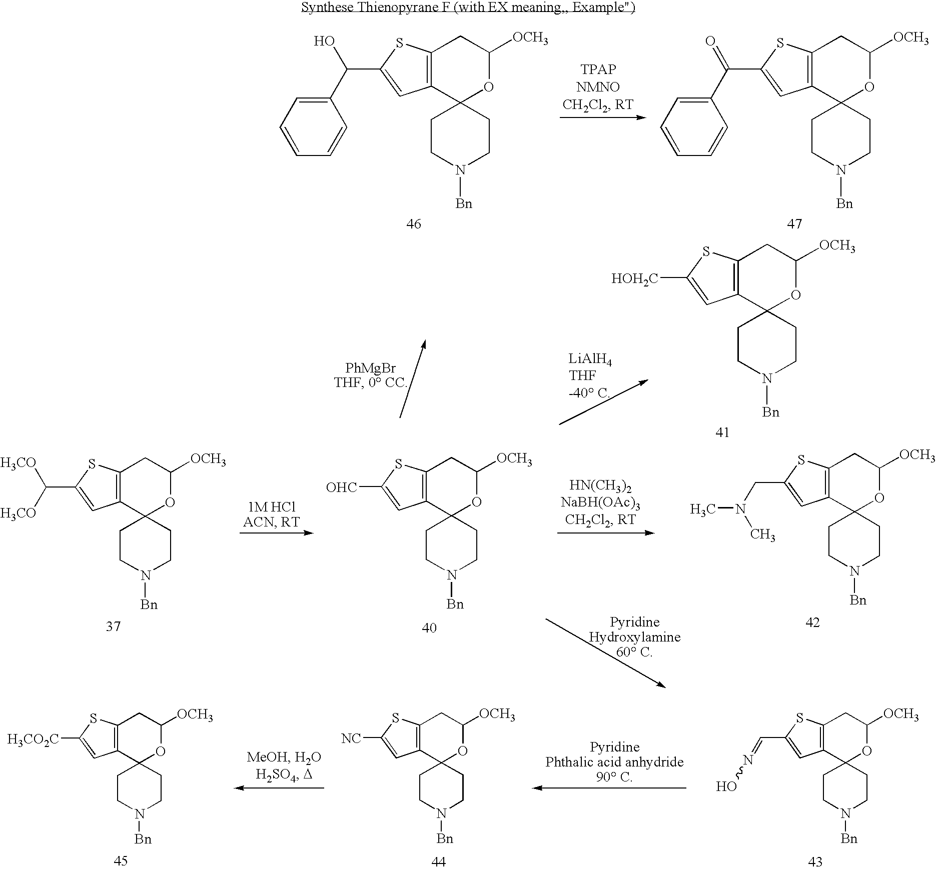

The present invention is illustrated below with the aid of examples. These illustrations are given solely by way of example and do not limit the general spirit of the present invention.

### EXAMPLES

### General Experimental Part (Methods and Equipment of the Synthesis and Analysis

All solvents used for synthesis were p. a. quality.

The separation of mixtures of substances using thin-layer chromatography was done on glass plates layered by silica gel. Analysis of the plates was done after treatment with iodine gas or incubation with Dragendorffs reagent under UV light.

As a rule synthesized products were purified using flash-chromatography.

Melting points were measured using capillaries. As sometimes the products were mixtures of diastereoisomers, the melting point: had to be expressed as a range.

IR-spectra were measured using the FT-IR-480 Plus Fourier Transform Spectrometer with ATR (Fa. Jasco). All substances were either measured directly as solids or in oil.

NMR-Spectra were measured using Mercury-400BB (Fa. Varian) at a temperature of 21° C. δ, measured in ppm, is based on the signal TMS measured in comparison to the residue signal (CHCl₃) of the solvent (CDCl₃):

¹H-NMR-Spectroscopy:

δ(TMS)=δ(CHCl₃)−7.26

¹³C-NMR-Spectroscopy:

δ(TMS)=δ(CHCl₃)−77.0

Mass-spectra (MS) were measured using the GCQ Finnigan MAT (Fa. Finnigan) with Xcalibur Version 1.1. The method of ionisation is shown in brackets: EI=electronic ionisation (70 eV); CI=Chemical ionisation (Isobutane or NH₃, 170 eV).

Elementary analysis was done with VarioEL (Fa. Elementar).

Before using HPLC the maximum absorption of the substances was measured using the UV/Vis-Spectra done with a 50Bio Cary Spektrophotometer (Fa. Varian).

For determination of the purity and for the separation of the diastereomers a HPLC Hitachi L6200A Intelligent Pump with a UV-Detector (Merck) was used.

Synthesis was done in the synthetic microwave Discover (Fa. CEM).

Some reactions were done using protective gas.

Reactions at −78° C. were done in an acetone bath in a Dewar.

### Example A

### 3-Bromthiophen-2-carbaldehyde

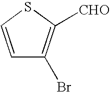

### Experimental Procedure

3-Bromo-thiophene (6.523 g, 40 mmol) was dissolved under N₂ in THF (100 mL). After cooling to 0-2° C. it was mixed dropwise with a 2M LDA solution (20 mL, 40 mmol) and stirred for 0.5 h. Following that 1-Formylpiperidin (4.52 g, 40 mmol) was added. After 2 h the reaction was stopped by adding a surplus of a saturated solution of NH₄Cl. For purification it was extracted three times with Et₂O (10 mL) and the pooled organic phases subsequently dried over Na₂SO₄. After filtration the solvent was removed under vacuum. The crude product (approx. 8.3 g) was purified using flash-chromatography (8 cm, cyclohexane:ethylacetate 9:1, 30 mL, R_{f}=0.65) and by distillation under vacuum (Bp. 62° C., 5.6·10⁻² mbar).

Colourless liquid, yield 6.05 g (79.2%).

C₅H₃BrOS (191.1)

MS (EI): m/z=190/192 [M⁺], 162 [M⁺-CHO].

IR (Film): v (cm⁻¹)=3103 (C—H), 2843 (C—H), 1657 (C═O).

¹H-NMR (CDCl₃):

δ (ppm)=7.15 (d, J=5.5 Hz, 1H, 4-H—Th), 7.71 (dd, J=5.1/1.5 Hz, 1H, 5-H—Th), 9.99 (d, J=1.5 Hz, 1H, Th—CHO).

### Example B

### cis/trans-3-Brom-2-(2-methoxyvinyl)-thiophene

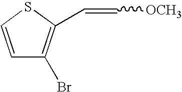

### Experimental Procedure

Methoxymethyltriphenylphosphoniumchloride (13.1 g, 38.3 mmol) was suspended in a 500 mL flask under N₂ in THF (200 ml). After cooling to T<−10° C. it was mixed with a 1M solution of potassium tertiary butylate (38.3 mL, 38.3 mmol) (change of colour to red). Few minutes later Example A (5.64 g, 29.5 mmol) was added (change of colour to yellow). After 2 h of reaction time at −10° C. purification followed. Thus, H₂O (80 mL) was added and three times extracted with Et₂O (15 mL). The pooled organic phases were dried over Na₂SO₄ and subsequently filtered. Then the solvent was removed under vacuum. Triphenylphosphaneoxide was separated from the crude product by extracting with a mixture of cyclohexane/ethylacetate. The crude product was purified by distillation under vacuum (Bp. 72° C. 5.6·10⁻² mbar).

Colourless liquid, yield: 5.39 g (83%).

C₇H₇BrOS (219.1).

MS (EI): m/z=218/220 [M⁺], 204 [M⁺-CH₃], 175 [M⁺-CH(OCH₃)].

IR (Film): v (cm⁻¹)=3008 (C═C—H), 2833 (C—H), 1634 (C═C), 1225 (C═C—O).

### cis-3-Brom-2-(2-methoxyvinyl)-thiophene

¹H-NMR (CDCl₃):

δ (ppm)=3.77 (s, 3H, OCH₃), 5.70 (dd, J=6.6/1.9 Hz, 1H, Th—CH═CHOCH₃), 6.18 (d, J=6.6 Hz, 1H, Th—CH═CHOCH₃), 6.85 (d, J=5.4 Hz, 1H, 4-H—Th), 7.09 (dd, J=5.4/0.9 Hz, 1H, 5-H—Th).

### trans3-Brom-2-(2-methoxyvinyl)-thiophene

¹H-NMR (CDCl₃):

δ (ppm)=3.64 (s, 3H, OCH₃), 5.89 (d, J=12.9 Hz, 1H, Th—CH═CH(OCH₃)), 6.82 (d, J=5.4 Hz, 1H, 4-H—Th), 6.89 (d, J=5.4 Hz, 1H, 5-H—Th), 6.97 (d, J=12.9 Hz, 1H, Th—CH═CHOCH₃).

Cis- and trans- are occurring in a ratio of 2:1.

### Example C

### 2-(3-Bromthiophen-2-yl)-acetaldehyddimethylacetal

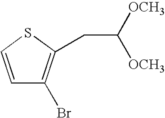

### Experimental Procedure

Example B (5.39 g, 24.6 mmol) was dissolved under N₂ in 80 mL MeOH. After addition of 10 mol % of p-toluene sulphonic acid (470 mg, 2.46 mmol) and trimethylorthoformiate (5 mL) it was heated for 24 h under reflux. For purification it was alkalised with 2M NaOH (15 mL) (pH=10) and H₂O (30 mL) added and then extracted three times with CH₂Cl₂ (10 mL). The pooled organic phases were dried over Na₂SO₄ and subsequently filtered. The solvent was removed under vacuum. Purification of crude product (approx. 5.8 g) was done by distillation under vacuum (Bp. 78° C., 5.6·10⁻² mbar).

Colourless liquid, yield 5.73 g (93%)).

C₈H₁₁BrO₂S (251.1).

MS (EI): m/z=251 [M⁺], 220 [M⁺-OCH₃], 140 [M⁺-Br, —OCH₃].

IR (Film): v (cm⁻¹)=3106 (C—H), 2830 (C—H), 1057 (C—O).

¹H-NMR (CDCl₃):

δ (ppm)=3.03 (d, J=5.6 Hz, 2H, Th—CH₂CH(OCH₃)₂), 3.31 (s, 6H, Th—CH₂CH(OCH₃)₂), 4.47 (t, J=5.6 Hz, 1H, Th—CH₂CH(OCH₃)₂), 6.84 (d, J=5.4 Hz, 1H, 4-H—Th), 7.15 (d, J=5.4 Hz, 1H, 5-H—Th).

### Example D

### Ethyl-4-[2-(2,2-dimethoxyethyl)-thiophen-3-yl]-4-hydroxypiperidin-1-carboxylat

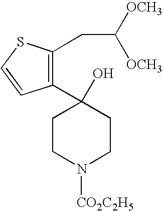

### Experimental Procedure

Example A (1.52 g, 6 mmol) was dissolved under N₂ in THF (80 mL) and cooled to −80° C. A solution of 1.35M n-butyllithium in n-hexane (5.2 mL, 7.8 mmol) was added dropwise within 2-3 min and stirred for 15 min. Following that 1-ethoxycarbonylypiperidin-4-one (1.02 g, 6.0 mmol), dissolved in THF (1.5 mL), was added and it was first stirred for one hour at −80° C. and subsequently for 3 h at RT. For purification H₂O (30 mL) was added and extracted three times with 10 mL CH₂Cl₂ extracted. The pooled organic phases were dried over Na₂SO₄ and subsequently filtered. The solvent was removed under vacuum. The crude product (approx. 2.22 g) was purified using flash-chromatography (4 cm, Cyclohexane:ethylacetate 7:3, 30 mL, R_{f}=0.3).

Yellowish oil, yield 1.48 g (approx. 70%, slightly polluted with some 1-ethoxy-carbonylpiperidin-4-one).

C₁₆H₂₅NO₅S (343.4).

MS (EI): m/z=343 [M⁺], 264 [M⁺-OH, —OCH₃ (2×)], 210 [M⁺-OCH₃ (2×), —COOC₂H₅].

IR (Film): v (cm⁻¹)=3436 (O—H), 2843 (C—H), 1671 (C═O), 1051 (C—O).

¹H-NMR (CDCl₃):

δ (ppm)=1.25 (t, J=7.2 Hz, 3H, CO₂CH₂CH₃), 1.65 (d, J=14.4 Hz, 2H, N(CH₂CH₂)₂), 1.87 (t, J=14.4 Hz, 2H, N(CH₂CH₂)₂), 3.09-3.25 (m, 4H, (Th—CH₂CH)(N(CH₂CH₂)₂)), 3.32 (s, 6H, CH(OCH₃)₂), 3.90-4.03 (m, 2H, N(CH₂CH₂)₂), 4.07 (q, J=7.2 Hz, 2H, CO₂CH₂CH₃), 4.43 (t, J=5.2 Hz, 1H, Th—CH₂CH), 6.73 (d, J=5.6 Hz, 1H, 4-H—Th), 7.11 (d, J=5.2 Hz, 1H, 5-H—Th).

### Example 1

### Ethyl-6′-methoxy-6′,7′-dihydrospiro[piperidine-4,4′-thieno[3,2-c]pyran]-1-carboxylate

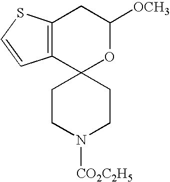

### Experimental Procedure

Example D (1.49 g, 4.3 mmol) was dissolved under N₂ in MeOH (50 mL). After addition of 1.2 equivalents of p-toluene sulphonic acid (1 g, 5.2 mmol) and trimethylorthoformiate (5 mL) it was stirred for 1 h at RT. For purification it was alkalised with 2M NaOH and H₂O (30 mL) added. Following that it was extracted three times with CH₂Cl₂ (10 mL). The pooled organic phases were dried over Na₂SO₄ and subsequently filtered. The solvent was removed under vacuum. The crude product (approx. 1.56 g) was purified using flash-chromatography (3 cm, cyclohexane:ethylacetate 7:3, 30 mL, R_{f}=0.54).

Colourless oil, yield 990 mg (53% over two steps).

C₁₅H₂₁NO₄S (311.4).

MS (EI): m/z=311 [M⁺], 280 [M⁺-OCH₃].

IR (Film): v (cm⁻¹)=2924 (C—H), 1693 (C═O), 1059 (C—O), 718 (C—H).

¹H-NMR (CDCl₃):

δ (ppm)=1.22 (t, J=7.2 Hz, 3H, CO₂CH₂CH₃), 1.69 (td, J=13.5/4.6 Hz, 1H, N(CH₂CH₂)₂), 1.76 (dd, J=13.5/4.6 Hz, 1H N(CH₂CH)₂), 1.83 (td, J=13.5/4.6 Hz, 1H, N(CH₂CH₂)₂), 1.89 (dd, J=13.5/4.6 Hz, 1H, N(CH₂CH₂)₂), 2.80 (dd, J=15.9/7.2 Hz, 1H, Th—CH₂), 2.95 (dd, J=15.9/3.3 Hz, 1H, Th—CH₂), 3.10-3.25 (m, 2H, N(CH₂CH₂)₂), 3.50 (s, 3H, OCH₃), 3.98-4.15 (m, 2H, N(CH₂CH₂)₂), 4.10 (q, J=7.2 Hz, 2H, CO₂CH₂CH₃), 4.83 (dd, J=7.2/3.3 Hz, 1H, Th—CH₂CH), 6.65 (d, J=5.1 Hz, 1H, 4-H—Th), 7.05 (d, J=5.1 Hz, 1H, 5-H—Th).

### Example 2

### 6′-Methoxy-6′,7′-dihydrospiro[piperidin-4,4′-thieno[3,2-c]pyran

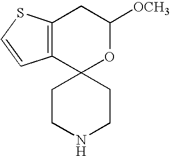

### Experimental Procedure

Example 1 (0.92 g, 2.96 mmol) was dissolved in a 100 mL flask in dioxane/H₂O 1:1 (50 mL). After addition of 70 equivalents of 2M NaOH (100 mL) it was heated for 5 h under reflux. For purification Et₂O (20 mL) was added and again extracted three times with Et₂O (5 mL). The pooled organic phases were dried over Na₂SO₄. Following filtration the solvent was removed under vacuum. The crude product (approx. 0.72 g) was purified using flash-chromatography (3 cm, MeOH:NH₃ 98:2, 15 mL, R_{f}=0.25).

yield: Colourless Solid, Mp. 103° C., yield 0.385 g (54%).

C₁₂H₁₇NO₂S (239.3).

MS (EI): m/z=239 [M⁺], 208 [M⁺-OCH₃], 151 (M⁺-OCH₃, —CH₂NHCH₂CH₂).

IR (Film): v (cm⁻¹)=3276 (N—H), 2915 (C—H), 1057 (C—O), 746 (C—H).

¹H-NMR (CDCl₃):

δ (ppm)=1.68 (td, J=14.0/4.7 Hz, 1H, N(CH₂CH₂)₂), 1.76 (dd, J=14.0/2.7 Hz, 1H, N(CH₂CH₂)₂), 1.84-1.96 (m, 2H, N(CH₂CH₂)₂), 2.78 (dd, J=15.6/7.5 Hz, 1H, Th—CH₂—CHOCH₃), 2.84-2.88 (m, 1H, N(CH₂CH₂)₂), 2.88-2.93 (m, 1H, N(CH₂CH₂)₂), 2.92 (dd, J=15.6/3.3 Hz, 1H, Th—CH₂CHOCH₃), 3.04 (td, J=12.3/2.7 Hz, 1H, N(CH₂CH₂)₂), 3.15 (td, J=12.3/3.0 Hz, 1H, N(CH₂CH₂)₂), 3.52 (s, 3H, Th—CH₂—CHOCH₃), 4.83 (dd, J=7.2/3.3 Hz, 1H, Th—CH₂—CHOCH₃), 6.72 (d, J=5.1 Hz, 1H, 4-H—Th), 7.03 (d, J=5.1 Hz, 1H, 5-H—Th). No signal for NH can be seen.

### Example E

### 2-[3-(1-Benzyl-4-hydroxypiperidin-4-yl)thiophen-2-yl]acetaldehyddimethylacetal

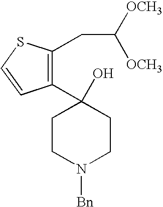

### Experimental Procedure

Example C (2.18 g, 8.7 mmol) was dissolved in THF (100 mL) and cooled under N₂ to −84° C. A solution of 1.35M in n-butyllithium in hexane (8.3 mL, 11.3 mmol) was added dropwise within 2-3 min under stirring, until after 15 min 1-benzyl piperidin-4-one (1.81 g, 9.57 mmol) was added. After 1 h of stirring at −84° C. a further 2 h it was stirred at RT. The reaction was stopped by adding 20 mL of water. Then a solution of NaHSO₃ (10 mL, 10%.) was added and the mixture extracted three times with 10 mL of CH₂Cl₂. The organic phases were pooled and dried over Na₂SO₄, followed by filtration and the solvent was removed under vacuum. The crude product (approx. 3.31 g) was purified using flash-chromatography (5 cm, cyclohexane:ethylacetate 7:3, 30 mL, R_{f}=0.3).

Yellowish oil, yield 2.14 g (68%).

C₂₀H₂₇NO₃S (361.5).

MS (EI): m/z=361 [M⁺], 330 [M⁺-OCH₃], 270 [M⁺-CH₂Ph], 91 [—CH₂Ph].

IR (Film): v (cm⁻¹)=3450 (O—H), 2851 (C—H), 1051 (C—O), 698 (C—H).

¹H-NMR (CDCl₃):

δ (ppm)=1.67 (dd, J=14.0/2.5 Hz, 2H, N(CH₂CH₂)₂), 2.04 (td, J=13.2/4.5 Hz, 2H, N(CH₂CH₂)₂), 2.45 (td, J=12.3/2.1 Hz, 2H, N(CH₂CH₂)₂), 2.62-2.71 (m, 2H, N(CH₂CH₂)₂), 3.30 (s, 6H, Th—CH₂—CH(OCH₃)₂), 3.38 (d, J=5.4 Hz, 2H, Th—CH₂—CH(OCH₃)₂), 3.50 (s, 2H, N—CH₂-Ph), 3.78 (s, 1H, OH), 4.44 (t, J=5.4 Hz, 1H, Th—CH₂—CH(OCH₃)₂), 6.83 (d, J=5.4 Hz, 1H, 4-H—Th), 6.99 (d, J=5.4 Hz, 1H, 5-H—Th) 7.10-7.21 (m, 5H, Ph-H).

### Example 3

### 1-Benzyl-6′-methoxy-6′,7′-dihydrospiro[piperidin-4,4′-thieno[3.2-c]pyran]

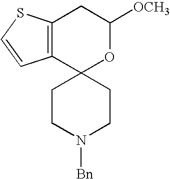

### Experimental Procedure

Example E (287 mg, 0.8 mmol) was dissolved in MeOH (15 mL) and mixed with trimethylorthoformiate (2 mL). After addition of p-toluene sulphonic acid (183 mg, 0.96 mmol) it was stirred for 24 h under N₂ 24 h at RT. For purification it was alkalised with 2M NaOH and H₂O (10 mL) added. Subsequently it was extracted three times with CH₂Cl₂ (5 mL). The organic phases were pooled and dried over Na₂SO₄, then it was filtered and the solvent removed under vacuum. The crude product (approx. 235 mg) was purified using flash-chromatography (2 cm, cyclohexane:ethylacetate 7:3, 15 mL, R_{f}=0.21).

Colourless crystalline solid, Mp. 78° C., yield 199 mg (75%).

C₁₉H₂₃NO₂S (329.5).

HPLC: Method 1: 95.0%, Rt=18.0 min
Method 2:

MS (EI): m/z=329 [M⁺], 298 [M⁺-OCH₃], 238 [M⁺-CH₂Ph], 91 [⁺CH₂Ph].

IR (Film): v (cm⁻¹)=3099 (C—H), 2810 (C—H), 1057 (C—O), 746 (C—H).

¹H-NMR (CDCl₃):

δ (ppm)=1.76 (dd, J=13.5/2.1 Hz, 1H, N(CH₂CH₂)₂), 1.80-1.93 (m, 2H, N(CH₂CH₂)₂), 2.04 (td, J=13.4/4.5 Hz, 1H, N(CH₂CH₂)₂), 2.37 (td, J=11.6/3.7 Hz, 1H, N(CH₂CH₂)₂), 2.45 (td, J=12.4/2.6 Hz, 1H, N(CH₂CH₂)₂), 2.65-2.73 (m, 2H, N(CH₂CH₂)₂), 2.77 (dd, J=15.6/7.5 Hz, 1H, Th—CH₂CHOCH₃), 2.92 (dd, J=15.6/3.3 Hz, 1H, Th—CH₂CHOCH₃), 3.48 (d, J=13.0 Hz, 1H, N—CH₂-Ph), 3.49 (s, 3H, Th—CH₂CHOCH₃), 3.54 (d, J=13.0 Hz, 1H, N—CH₂-Ph), 4.81 (dd, J=7.2/3.3 Hz, 1H, Th—CH₂CHOCH₃), 6.73 (d, J=5.4 Hz, 1H, 4-H—Th), 7.02 (d, J=5.4 Hz, 1H, 5-H—Th), 7.15-7.31 (m, 5H, Ph-H).

### Example 2

### 6′-Methoxy-6′,7′-dihydrospiro[piperidin-4,4′-thieno[3,2-c]pyran (Different Approach)

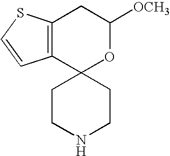

### Experimental Procedure

Example 3 (100 mg, 0.31 mmol) was dissolved in THF (8 mL) and cooled to −78° C. under N₂. After addition of chloro-formic-acid-chloroethylester (45 μL, 0.40 mmol) it was stirred for 20 min, then heated to RT and THF removed under vacuum. Following that the product was mixed with MeOH (8 mL) and heated for 40 min under reflux. Subsequently the methanol was removed under vacuum. The crude product (approx. 75 mg) was purified using flash-chromatography (0.7 cm, ethyl-acetate:MeOH:NH₃ 90:10:2, 3 mL, R_{f}=0.18).

Colourless solid, Mp. 103° C., yield 48 mg (65%).

### Example F

### 2-{3-[4-Hydroxy-1-(2-phenylethyl)piperidin-4-yl]thiophen-2yl}acetaldehyd-dimethylacetal

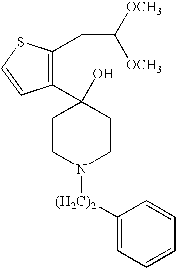

### Experimental Procedure

Example C (0.5 g, 2 mmol) was dissolved in THF (25 mL) and cooled to −80° C. under N₂. A solution of 1.35M n-butyllithium in hexane (1.7 mL, 2.4 mmol) was added dropwise within 2-3 min while stirring, until after 15 min 1-(2-Phenylethyl)piperidin-4-one (450 mg, 2.2 mmol) was added. After 1.5 h of stirring at −80° C. a further 1.5 h of stirring at RT followed. The reaction was stopped by addition of H₂O (10 mL). Following that a solution of NaHSO₃ (10 mL, 10%) was added and extracted three times with CH₂Cl₂ (5 mL). The organic phases were pooled and dried over Na₂SO₄. Subsequently it was filtered and the solvent removed under vacuum. The crude product (approx. 1 g) was purified using flash-chromatography (3 cm, cyclo-hexane:ethylacetate 7:3, 30 mL, R_{f}=0.18).

Yellowish oil, yield 62 mg (8%).

C₂₁H₂₉NO₃S (375.5).

MS (EI): m/z=375 [M⁺], 344 [M⁺-OCH₃], 284 [M⁺-CH₂Ph], 253 [M⁺-OCH₃, —CH₂Ph], 91 [⁺CH₂Ph].

IR (Film): v (cm⁻¹)=3444 (O—H); 2922, 2828 (C—H); 1070, 1055 (C—O).

¹H-NMR (CDCl₃):

δ (ppm)=1.69-1.81 (m, 4H(N(CH₂CH₂)₂)(N—CH₂CH₂Ph)), 2.10 (td, J=13.3/3.4 Hz, 2H, N(CH₂CH₂)₂), 2.46-2.67 (m, 2H, N(CH₂CH₂)₂), 2.75-2.86 (m, 4H(N—CH₂CH₂Ph) (N(CH₂CH₂)₂)), 3.32 (s, 6H, Th—CH₂CH(OCH₃)₂), 3.39 (d, J=5.4 Hz, 2H, Th—CH₂CH(OCH₃)₂), 3.90 (s, 1H, —OH), 4.45 (t, J=5.4 Hz, 1H, Th—CH₂CH(OCH₃)₂), 6.85 (d, J=5.4 Hz, 1H, 4-H—Th), 7.01 (d, J=5.4 Hz, 1H, 5-H—Th), 7.13-7.25 (m, 5H, Ph-H).

### Example 4

### 6′-Methoxy-1-(2-phenylethyl)-6′,7′-dihydrospiro[piperidin-4,4′-thieno[3.2-c]pyran]

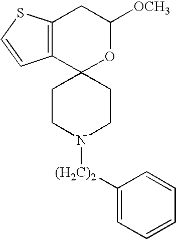

### Experimental Procedure

Example F (55 mg, 0.14 mmol) was dissolved in MeOH (5 mL) and after addition of trimethylorthoformiate (1 mL) mixed with p-toluene sulphonic acid (33 mg, 1.75 mmol). After stirring for 24 h at RT it was alkalised with 2M NaOH (2 mL) and extracted three times with CH₂Cl₂ (5 mL). The pooled organic phases were dried over Na₂SO₄, then filtered and the solvent removed under vacuum. The crude product (approx. 50 mg) was purified using flash-chromatography (1 cm, cyclohexane:ethylacetate 7:3, 5 mL, R_{f}=0.10).

Beige crystalline solid, mp. 94° C., yield 21 mg (44%).

C₂₀H₂₅NO₂S (343.5).

HPLC: Method 1: 97.9%, Rt=18.9 min
Method 2:

MS (EI): m/z=343 [M⁺], 312 [M⁺-OCH₃], 252 [M⁺-CH₂Ph], 91 [⁺CH₂Ph].

IR (Film): v (cm⁻¹)=3108 (C—H), 2954 (C—H), 1060 (C—O), 747 (C—H).

¹H-NMR (CDCl₃):

δ (ppm)=1.78-1.98 (m, 3H, N(CH₂CH₂)₂), 2.08 (td, J=12.6/4.4 Hz, 1H, N(CH₂CH₂)₂), 2.38-2.56 (m, 2H, N(CH₂CH₂)₂), 2.57-2.63 (m, 2H, N—CH₂CH₂-Ph), 2.76-2.89 (m, 5H, (Th—CH₂CHOCH₃)(N—CH₂CH₂-Ph) (N(CH₂CH₂)₂)), 2.94 (dd, J=15.6/3.3 Hz, 1H, Th—CH₂CHOCH₃), 3.51 (s, 3H, Th—CH₂CHOCH₃), 4.82 (dd, J=7.2/3.3 Hz, 1H, Th—CH₂CHOCH₃), 6.75 (d, J=5.4 Hz, 1H, 4-H—Th), 7.04 (d, J=5.4 Hz, 1H, 5-H—Th), 7.15-7.28 (m, 5H, Ph-H).

### Example G

### 2-{3-[1-(Cyclohexylmethyl)-4-hydroxypiperidin-4-yl]thiophen-2-yl}acetaldehyddimethylacetal

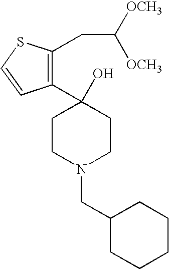

### Experimental Procedure

Example C (1.18 g, 4.7 mmol) was dissolved in THF (50 mL) and cooled to −78° C. under N₂. A solution of 1.5M n-butyllithium in hexane (5.2 mL, 7.0 mmol) was added dropwise within 2-3 min while stirring, until after 15 min 1-(Cyclohexylmethyl)piperidin-4-one (1.0 g, 5.2 mmol) was added. After stirring for 1.5 h at −78° C. it was stirred a further 2 h at RT. The reaction was stopped by addition of H₂O (50 mL). Following that it was extracted three times with CH₂Cl₂ (10 mL). The organic phases were pooled and dried over Na2SO4, then filtered and the solvent removed under vacuum. The crude product (approx. 1.8 g) was purified using flash-chromatography (4 cm, Cyclohexane:ethylacetate 1:1, 30 mL, R_{f}=0.21).

Colourless oil, yield 0.8 g (46%).

C₂₀H₃₃NO₃S (367.5).

MS (EI): m/z=367 [M⁺], 336 [M⁺-OCH₃], 284 [M⁺-cHex].

IR (Film): v (cm⁻¹)=3457 (O—H), 2918 (C—H), 1053 (C—O), 719 (C—H).

¹H-NMR (CDCl₃):

δ(ppm)=0.77-0.87 (m, 2H, cHex-H), 1.07-1.20 (m, 3H, cHex-H), 1.40-1.48 (m, 1H, cHex-H), 1.55-1.75 (m, 5H, cHex-H), 1.66 (dd, J=13.7/2.6 Hz, 2H, N(CH₂CH₂)₂), 2.03 (td, J=12.6/3.3 Hz, 2H, N(CH₂CH₂)₂), 2.11 (d, J=7.4 Hz, 2H, N—CH₂-cHex), 2.31 (t, J=11.7 Hz, 2H, N(CH₂CH₂)₂), 2.59-2.65 (m, 2H, N(CH₂CH₂)₂), 3.29 (s, 6H, Th—CH₂CH(OCH₃)₂), 3.87 (d, J=4.2 Hz, 2H, Th—CH₂CH(OCH₃)₂), 3.69 (s, 1H, OH), 4.44 (t, J=4.2 Hz, 1H, Th—CH₂CH(OCH₃)₂), 6.83 (d, J=5.2 Hz, 1H, 4-H—Th), 7.00 (d, J=5.2 Hz, 1H, 5-H—Th).

### Example 5

### 1-(Cyclohexylmethyl)-6′Methoxy-6′,7′-dihydrospiro[piperidin-4,4′-thieno[3.2-c]pyran]

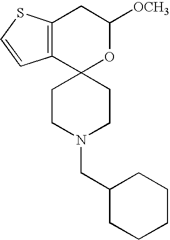

### Experimental Procedure

Example G (0.8 g, 2.2 mmol) was dissolved in MeOH (30 mL) and mixed with trimethyl orthoformiate (3 mL). Following addition of p-toluene sulphonic acid (0.5 g, 2.6 mmol) it was stirred for 3 h at RT under N₂. For purification it was alkalised with 2M NaOH and H₂O (10 mL) added. Subsequently it was extracted three times with CH₂Cl₂ (10 mL) extracted. The organic phases were pooled and dried over Na₂SO₄, then filtered and the solvent removed under vacuum. The crude product was purified using flash-chromatography (2.5 cm, Cyclohexane:ethylacetate 7:3, 15 mL, R_{f}=0.45).

Colourless crystalline solid, Mp. 78° C., yield 490 mg (68%).

### Example H

### 2-{3-[4-Hydroxy-1-(3-methylbutyl)piperidin-4-yl]thiophen-2-yl}acetaldehyddimethylacetal

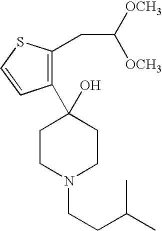

### Experimental Procedure

Example C (1.6 g, 6.4 mmol) was dissolved in THF (80 mL) and cooled to −78° C. under N₂. A solution of 1.5M n-butyllithium in hexane (5.15 mL, 7.7 mmol) was added dropwise within 2-3 min while stirring, until after 15 min 1-(3-Methylbutyl)piperidin-4-one (1.2 g, 7.0 mmol) was added. After 2 h of stirring at −78° C. another 2 h of stirring at RT was added. The reaction was stopped by addition of H₂O (50 mL). Subsequently it was extracted three times with CH₂Cl₂ (10 mL) extracted. The organic phases were pooled and dried over Na2SO4, then filtered and the solvent removed under vacuum. The crude product (approx. 2.2 g) was purified using flash-chromatography (4 cm, Cyclohexane:ethylacetate 1:1, 50 mL, R_{f}=0.22).

Colourless oil, yield 1.2 g (approx. 55%, slight impurities of 1-(3-Methylbutyl)piperidin-4-one).

C₁₈H₃₁NO₃S (341.5).

MS (EI): m/z=341 [M⁺], 310 [M⁺-OCH₃], 284 [M⁺-CH₂CH(CH₃)₂].

IR (Film): v (cm⁻¹)=3446 (O—H), 2951 (C—H), 1054 (C—O), 719 (C—H).

¹H-NMR (CDCl₃):

δ (ppm)=0.91 (d, J=4.8 Hz, 6H, CH₂CH₂CH(CH₃)₂), 1.41-1.45 (m, 1H, CH₂CH₂CH(CH₃)₂), 1.52-1.62 (m, 2H, CH₂CH₂CH(CH₃)₂), 1.76 (dd, J=13.7/2.7 Hz, 2H, N(CH₂CH₂)₂), 2.13 (td, J=12.6/4.2 Hz, 2H, N(CH₂CH₂)₂), 2.38-2.48 (m, 4H(CH₂CH₂CH(CH₃)₂)(N(CH₂CH₂)₂)), 2.77-2.82 (m, 2H, N(CH₂CH₂)₂), 3.37 (s, 6H, Th—CH₂CH(OCH₃)₂), 3.45 (d, J=4.2 Hz, 2H, Th—CH₂CH(OCH₃)₂), 3.85 (s, 1H, OH), 4.51 (t, J=4.2 Hz, 1H, Th—CH₂CH(OCH₃)₂), 6.90 (d, J=5.2 Hz, 1H, 4-H—Th), 7.06 (d, J=5.2 Hz, 1H, 5-H—Th).

### Example 6

### 6′-Methoxy-1-(3-methylbutyl)-6′,7′-dihydrospiro[piperidin-4,4′-thieno[3.2-c]pyran]

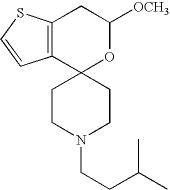

### Experimental Procedure

Example H (1.2 g, 3.5 mmol) was dissolved in MeOH (40 mL) and mixed with trimethylorthoformiate (4 mL). After addition of p-toluene sulphonic acid (0.8 g, 4.2 mmol) it was stirred under N₂ for 24 h at RT. For purification it was alkalised with 2M NaOH and H₂O (10 mL) added. Subsequently it was extracted three times with CH₂Cl₂ (10 mL). The organic phases were pooled and dried over Na2SO4, then filtered and the solvent removed under vacuum. The crude product (approx. 1.1 g) was purified using flash-chromatography (4 cm, cyclohexane:ethylacetate 7:3, 30 mL, R_{f}=0.13).

Colourless crystalline solid, Mp. 78° C., yield 950 mg (88%).

### Example 5

### 1-(Cyclohexylmethyl)-6′Methoxy-6′,7′-dihydrospiro[piperidin-4,4-thieno[3.2-c]pyran]] (alternative route)

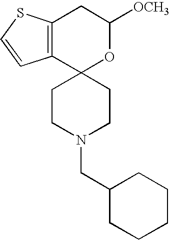

### Experimental Procedure

Example 2 (90 mg, 0.37 mmol) was dissolved in CH₃CN (8 mL), mixed with cyclohexylmethylbromide (80 mg, 0.45 mmol) and K₂CO₃ (300 mg) and heated for 24 h under reflux. For purification K₂CO₃ was removed by a frit and the solution transferred into a separating funnel. After addition of saturated NaCl solution (10 mL) it was extracted three times with Et₂O (5 mL). The organic phases were pooled and dried over Na2SO4, then filtered and the solvent removed under vacuum. The crude product (approx. 162 mg) was purified using flash-chromatography (1.5 cm, Cyclohexane:ethylacetate 7:3, 10 mL, R_{f}=0.45).

Colourless crystalline solid, Mp. 77° C., yield 118 mg (95%).

C₁₉H₂₉NO₂S (335.5).

HPLC: Method 1: 98.5%, R=19.1 min
Method 2:

MS (EI): m/z=335 [M⁺], 304 [M⁺-OCH₃], 252 [M⁺-cHex].

IR (Film): v (cm⁻¹)=3102 (C—H), 2914 (C—H), 1058 (C—O), 748 (C—H).

¹H-NMR (CDCl₃):

δ (ppm)=0.82-0.87 (m, 2H, cHex-H), 1.12-1.20 (m, 2H, cHex-H), 1.41-1.51 (m, 1H, cHex-H), 1.56-1.86 (m, 9H(N(CH₂CH₂)₂) (cHex-H), 2.05 (td, J=13.2/4.5 Hz, 1H, N(CH₂CH₂)₂), 2.12 (d, J=7.0 Hz, 2H, N—CH₂-cHex), 2.27 (td, J=12.1/3.3 Hz, 1H, N(CH₂CH₂)₂), 2.34 (td, J=12.5/2.3 Hz, 1H, N(CH₂CH₂)₂), 2.64-2.70 (m, 2H, N(CH₂CH₂)₂), 2.78 (dd, J=16.0/7.1 Hz, 1H, Th—CH₂CHOCH₃), 2.92 (dd, J=15.6/3.1 Hz, 1H, Th—CH₂CHOCH₃), 3.51 (s, 3H, Th—CH₂CHOCH₃), 4.81 (dd, J=7.4/3.1 Hz, 1H, Th—CH₂CHOCH₃), 6.72 (d, J=5.1 Hz, 1H, 4-H—Th), 7.02 (d, J=5.5 Hz, 1H, 5-H—Th).

### Example 6

### 6′-Methoxy-1-(3-methylbutyl)-6′,7′-dihydrospiro[piperidin-4,4′-thieno[3.2-c]pyran] (alternative route)

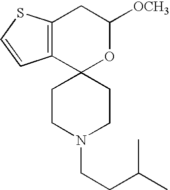

### Experimental Procedure

Example 2 (58 mg, 0.24 mmol) was dissolved in CH₂Cl₂ (5 mL), mixed with Isovaleraldehyde (22 mg, 0.25 mmol) and NaBH(OAc)₃ (80 mg, 0.38 mmol) and stirred for 2 h at RT. Following the end of the reaction a saturated solution of NaHCO₃ (5 mL) was added and extracted three times with CH₂Cl₂ (5 mL). The organic phases were pooled and dried over Na2SO4, then filtered and the solvent removed under vacuum. The crude product (approx. 100 mg) was purified using flash-chromatography (1 cm, cyclohexane:ethylacetate 7:3, 5 mL, R_{f}=0.13).

Colourless crystalline solid, Mp. 38° C., yield 61.5 mg (82.5%).

C₁₇H₂₇NO₂S (309.5).

HPLC: Method 1: 98.3%, R=18.1 min
Method 2:

MS (EI): m/z=309 [M⁺], 278 [M⁺-OCH₃], 252 [M⁺-C₄H₉].

IR (Film): v (cm⁻¹)=3098 (C—H), 2949 (C—H), 1058 (C—O), 745 (C—H).

¹H-NMR (CDCl₃):

δ (ppm)=0.85 (d, J=6.7 Hz, 6H, N—CH₂CH₂CH(CH₃)₂), 1.36-1.41 (m, 1H, N—CH₂CH₂CH(CH₃)₂), 1.51-1.58 (m, 2H, N—CH₂CH₂CH(CH₃)₂), 1.78 (dd, J=13.7/2.7 Hz, 1H, N(CH₂CH₂)₂), 1.84 (td, J=12.6/4.3 Hz, 1H, N(CH₂CH₂)₂), 1.92 (dd, J=14.2/2.8 Hz, 1H, N(CH₂CH₂)₂), 2.05 (td, J=13.2/4.5 Hz, 1H, N(CH₂CH₂)₂), 2.27-2.42 (m, 4H(N—CH₂CH₂CH(CH₃)₂) (N(CH₂CH₂)₂)), 2.74-2.81 (m, 3H, (N(CH₂CH₂)₂) (Th—CH₂CHOCH₃), 2.91 (dd, J=15.6/3.3 Hz, 1H, Th—CH₂CHOCH₃), 3.51 (s, 3H, Th—CH₂CH(OCH₃), 4.82 (dd, J=7.1/3.3 Hz, 1H, Th—CH₂CHOCH₃), 6.74 (d, J=5.1 Hz, 1H, 4-H—Th), 7.03 (d, J=5.1 Hz, 1H, 5-H—Th).

### Example 7

### 6′-Methoxy-1-(3-methylbut-2-enyl)-6′,7′-dihydrospiro[piperidin-4.4-thieno[3.2-c]pyran]

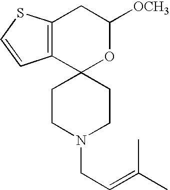

### Experimental Procedure

Example 2 (59 mg, 0.24 mmol) was dissolved in CH₃CN (5 mL), mixed with 1-Bromo-3-methyl-2-butene (45 mg, 0.3 mmol) and K₂CO₃ (200 mg) and heated for 24 h under reflux. For purification K₂CO₃ was removed by a frit. After addition of saturated NaCl-solution (5 mL) it was extracted three times with Et₂O (5 mL). The organic phases were pooled and dried over Na2SO4, then filtered and the solvent removed under vacuum. The crude product (approx. 100 mg) was purified using flash-chromatography (1 cm, cyclohexane:ethylacetate 7:3, 5 mL, R_{f}=0.15).

Colourless crystalline solid, Mp. 40° C., yield: 16 mg (21%).

C₁₇H₂₅NO₂S (307.5).

HPLC: Method 1: 97.5%, Rt=17.8 min
Method 2:

MS (EI): m/z=307 [M⁺], 276 [M⁺-OCH₃], 239 [M⁺-CH₂CH═C(CH₃)₂].

IR (Film): v (cm⁻¹)=3096 (C—H), 2813 (C—H), 1675 (C═C), 1058 (C—O), 744 (C—H).

¹H-NMR (CDCl₃):

δ (ppm)=1.60 (s, 3H, N—CH₂CH═C(CH₃)₂), 1.68 (s, 3H, N—CH₂CH═C(CH₃)₂), 1.79 (dd, J=13.7/2.7 Hz, 1H, N(CH₂CH₂)₂), 1.84 (td, J=12.5/4.5 Hz, 1H, N(CH₂CH₂)₂), 1.91 (dd, J=13.7/2.5 Hz, 1H, N(CH₂CH₂)₂), 2.05 (td, J=13.2/4.4 Hz, 1H, N(CH₂CH₂)₂), 2.32 (td, J=12.0/3.3 Hz, 1H, N(CH₂CH₂)₂), 2.41 (td, J=12.6/2.6 Hz, 1H, N(CH₂CH₂)₂), 2.76-2.81 (m, 3H(N(CH₂CH₂)₂) (Th—CH₂CHOCH₃), 2.90-2.97 (m, 3H, (Th—CH₂CHOCH₃)(N—CH₂CH═C(CH₃)₂)), 3.52 (s, 3H, Th—CH₂CHOCH₃), 4.82 (dd, J=7.1/3.3 Hz, 1H, Th—CH₂CHOCH₃), 5.25 (t, J=7.8 Hz, 1H, N—CH₂CH═C(CH₃)₂), 6.73 (d, J=5.1 Hz, 1H, 4-H—Th), 7.03 (d, J=5.1 Hz, 1H, 5-H—Th).

### Example 8

### 1-Butyl-6′-methoxy-6′,7′-dihydrospiro[piperidin-4,4′-thieno[3.2-c]pyran]

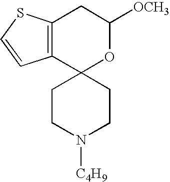

### Experimental Procedure

Example 2 (90 mg, 0.37 mmol) was dissolved in CH₃CN (8 mL), mixed with butylbromide (79 mg, 0.57 mmol) and K₂CO₃ (300 mg) and heated for 24 h under reflux. For purification K₂CO₃ was removed over a frit, and the solution transferred to separating funnel. After addition of a saturated NaCl-solution (10 mL) it was extracted three times with Et₂O (5 mL). The organic phases were pooled and dried over Na₂SO₄, then filtered and the solvent removed under vacuum. The crude product (approx. 110 mg) was purified using flash-chromatography (1 cm, Cyclohexane:ethylacetate 7:3, 10 mL, R_{f}=0.14).

Colourless crystalline solid, Mp. 47° C., yield 70 mg (65%).

C₁₆H₂₅NO₂S (295.4).

HPLC: Method 1: 97.4%, Rt=16.9 min
Method 2:

MS (EI): m/z=295 [M⁺], 264 [M⁺-OCH₃], 252 [M⁺-C₃H₇].

IR (Film): v (cm⁻¹)=2926 (C—H); 2807 (N—C—H), 1060 (C—O), 719 (C—H).

¹H-NMR (CDCl₃):

δ (ppm)=0.94 (t, J=7.2 Hz, 3H, N—CH₂CH₂CH₂CH₃), 1.36 (sext, J=7.2 Hz, 2H, N—CH₂CH₂CH₂CH₃), 1.54 (quin, J=7.2 Hz, 2H, N—CH₂CH₂CH₂CH₃), 1.85 (dd, J=13.7/2.6 Hz, 1H, N(CH₂CH₂)₂), 1.93 (td, J=14.2/4.4 Hz, 1H, N(CH₂CH₂)₂), 1.99 (dd, J=13.7/2.6 Hz, 1H, N(CH₂CH₂)₂), 2.14 (td, J=13.2/4.5 Hz, 1H, N(CH₂CH₂)₂), 2.37-2.45 (m, 3H, (N(CH₂CH₂)₂)(CH₂CH₂CH₂CH₃)), 2.49 (td, J=12.4/2.6 Hz, 1H, N(CH₂CH₂)₂), 2.80-2.89 (m, 3H(N(CH₂CH₂)₂) (Th—CH₂CHOCH₃)), 2.99 (dd, J=15.6/3.3 Hz, 1H, Th—CH₂CHOCH₃), 3.57 (s, 3H, Th—CH₂CHOCH₃), 4.88 (dd, J=7.8/3.3 Hz, 1H, Th—CH₂CHOCH₃), 6.80 (d, J=5.1 Hz, 1H, 4-H—Th), 7.08 (d, J=5.4 Hz, 1H, 5-H—Th).

### Example 9

### 6′-Methoxy-1-pentyl-6′,7′-dihydrospiro[piperidin-4,4′-thieno[3.2-c]pyran]

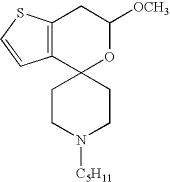

### Experimental Procedure

Example 2 (62 mg, 0.26 mmol) was dissolved in CH₂Cl₂ (5 mL), mixed with valeraldehyde (24 mg, 0.28 mmol) and NaBH(OAc)₃ (80 mg, 0.38 mmol) and stirred for 2 h at RT. After the finishing of the reaction saturated NaHCO₃-solution (5 mL) was added and three times extracted with CH₂Cl₂ (5 mL). The organic phases were pooled and dried over Na2SO4, then filtered and the solvent removed under vacuum. The crude product (approx. 100 mg) was purified using flash-chromatography (1 cm, cyclohexane:ethylacetate 7:3, 5 mL, R_{f}=0.2).

Colourless crystalline solid, Mp. 43° C., yield 69 mg (88%).

C₁₇H₂₇NO₂S (309.5).

HPLC: Method 1: 99.1%, Rt=18.4 min
Method 2:

MS (EI): m/z=309 [M⁺], 278 [M⁺-OCH₃], 252 [M⁺-C₄H₉].

IR (Film): v (cm⁻¹)=3098 (C—H), 2926 (C—H), 1058 (C—O), 744 (C—H).

¹H-NMR (CDCl₃):

δ (ppm)=0.84 (t, J=6.6 Hz, 3H, N—CH₂CH₂CH₂CH₂CH₃), 1.23-1.28 (m, 4H, N—CH₂CH₂CH₂CH₂CH₃), 1.42-1.52 (m, 2H, N—CH₂CH₂CH₂CH₂CH₃), 1.78 (dd, J=13.7/2.7 Hz, 1H, N(CH₂CH₂)₂), 1.84 (td, J=12.6/4.3 Hz, 1H, N(CH₂CH₂)₂), 1.92 (dd, J=14.2/2.8 Hz, 1H, N(CH₂CH₂)₂), 2.05 (td, J=13.2/4.4 Hz, 1H, N(CH₂CH₂)₂), 2.28-2.35 (m, 3H(N—CH₂CH₂CH₂CH₂CH₃)(N(CH₂CH₂)₂)), 2.39 (td, J=12.6/2.6 Hz, 1H, N(CH₂CH₂)₂), 2.73-2.81 (m, 3H(N(CH₂CH₂)₂) (Th—CH₂CHOCH₃), 2.92 (dd, J=15.6/3.2 Hz, 1H, Th—CH₂CHOCH₃), 3.51 (s, 3H, Th—CH₂CHOCH₃), 4.82 (dd, J=7.4/3.1 Hz, 1H, Th—CH₂CHOCH₃), 6.74 (d, J=5.1 Hz, 1H, 4-H—Th), 7.03 (d, J=5.5 Hz, 1H, 5-H—Th).

### Example 10

### 6′-Methoxy-1-octyl-6′,7′-dihydrospiro[piperidin-4,4-thieno[3.2-c]pyran]

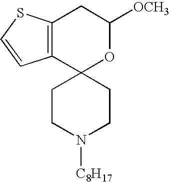

### Experimental Procedure

Example 2 (180 mg, 0.75 mmol) was dissolved in CH₃CN (20 mL), mixed with octylbromide (174 mg, 0.9 mmol) and K₂CO₃ (620 mg) and heated for 24 h under reflux. For purification K₂CO₃ was separated over a frit and the solution transferred into a separating funnel. After addition of saturated NaCl-solution (10 mL) it was extracted three times with Et₂O (5 mL). The organic phases were pooled and dried over Na₂SO₄, then filtered and the solvent removed under vacuum. The crude product (approx. 237 mg) was purified using flash-chromatography (2 cm, cyclohexane:ethylacetate 7:3, 10 mL, R_{f}=0.37).

Colourless crystalline solid, Mp. 48° C., yield 229 mg (87%).

C₂₀H₃₃NO₂S (351.1).

HPLC: Method 1: 99.1%, Rt=21.8 min
Method 2:

MS (EI): m/z=351 [M⁺], 320 [M⁺-OCH₃], 252 [M⁺-C₇H₁₅].

IR (Film): v (cm⁻¹)=3092 (C—H), 2918 (C—H), 1058 (C—O), 742 (C—H).

¹H-NMR (CDCl₃):

δ (ppm)=0.81 (t, J=6.0 Hz, 3H, N—(CH₂)₇—CH₃), 1.12 (m, 10H, N—CH₂CH₂(CH₂)₅CH₃), 1.43 (m, 2H, N—CH₂CH₂C₆H₁₃), 1.78 (dd, J=13.5/2.2 Hz, 1H, N(CH₂CH₂)₂), 1.88 (td, J=12.2/4.2 Hz, 1H, N(CH₂CH₂)₂), 1.93 (dd, J=13.5/2.2 Hz, 1H, N(CH₂CH₂)₂), 2.05 (td, J=13.2/4.5 Hz, 1H, N(CH₂CH₂)₂), 2.25-2.45 (m, 4H(N(CH₂CH₂)₂)(N—CH₂(CH₂)₆CH₃)), 2.77 (dd, J=15.3/7.2 Hz, 1H, Th—CH₂CHOCH₃), 2.75-2.80 (m, 2H, N(CH₂CH₂)₂), 2.94 (dd, J=15.3/3.3 Hz, 1H, Th—CH₂CHOCH₃), 3.51 (s, 3H, Th—CH₂CHOCH₃), 4.82 (dd, J=7.2/3.3 Hz, 1H, Th—CH₂—CHOCH₃), 6.73 (d, J=5.4 Hz, 1H, 4-H—Th), 7.02 (d, J=5.4 Hz, 1H, 5-H—Th).

### Example 11

### 3′-Methoxy-1-(3-phenylpropyl)-6′,7′-dihydrospiro[piperidin-4,4′-thieno[3.2-c]pyran]

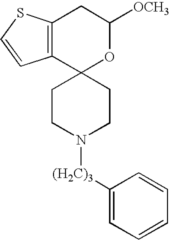

### Experimental Procedure

Example 2 (120 mg, 0.5 mmol) was dissolved in CH₃CN (15 mL), mixed with 3-phenylpropylbromide (120 mg, 0.6 mmol) and K₂CO₃ (414 mg) and heated for 24 h under reflux. For purification K₂CO₃ was removed by a frit and the solution transferred into a separating funnel. After addition of saturated NaCl-solution (10 mL) it was extracted three times with Et₂O. The organic phases were pooled and dried over Na2SO4, then filtered and the solvent removed under vacuum. The crude product was purified using flash-chromatography (2 cm, cyclohexane:ethylacetate 7:3, 10 mL, R_{f}=0.2).

Colourless Solid, Mp. 59° C., yield 68 mg (38%).

C₂₁H₂₇NO₂S (357.5).

HPLC: Method 1: %, Rt=min
Method 2:

MS (EI): m/z=357 [M⁺], 326 [M⁺-OCH₃], 252 [M⁺-(CH₂)₂-Ph].

IR (Film): v (cm⁻¹)=3097 (C—H), 2913 (C—H), 1062 (C—O), 692 (C—H).

¹H-NMR (CDCl₃):

δ (ppm)=1.75-1.89 (m, 4H(N(CH₂CH₂)₂)(N—CH₂CH₂CH₂-Ph)), 1.92 (dd, J=13.8/2.8 Hz, 1H, N(CH₂CH₂)₂), 2.04 (td, J=13.8/4.2 Hz, 1H, N(CH₂CH₂)₂), 2.26-2.46 (m, 4H(N(CH₂CH₂)₂)(N—CH₂CH₂CH₂-Ph)), 2.59 (t, J=7.8 Hz, 2H, N—CH₂CH₂CH₂-Ph), 2.71-2.79 (m, 2H, N(CH₂CH₂)₂), 2.77 (dd, J=15.6/7.2 Hz, 1H, Th—CH₂CHOCH₃), 2.92 (dd, J=15.6/3.3 Hz, 1H, Th—CH₂CHOCH₃), 3.50 (s, 3H, Th—CH₂CHOCH₃), 4.81 (dd, J=7.2/3.3 Hz, 1H, Th—CH₂CHOCH₃), 6.73 (d, J=5.1 Hz, 1H, 4-H—Th), 7.02 (d, J=5.4 Hz, 1H, 5-H—Th), 7.10-7.25 (m, 5H, Ph-H).

### Example 12

### 3′-Methoxy-1-(4-phenylbutyl)-6′,7′-dihydrospiro[piperidin-4,4% thieno[3.2-c]pyran]

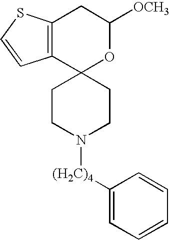

### Experimental Procedure

Example 2 (89 mg, 0.36 mmol) was dissolved in CH₃CN (10 mL), mixed with 4-phenylbutylchloride (76 mg, 0.45 mmol) and K₂CO₃ (310 mg) and heated for 24 h under reflux. For purification K₂CO₃ was removed by a frit and the solution transferred into a separating funnel. After addition of saturated NaCl-solution (10 mL) it was extracted three times with Et₂O. The organic phases were pooled and dried over Na₂SO₄, then filtered and the solvent removed under vacuum. The crude product (approx. 195 mg) was purified using flash-chromatography (1.5 cm, Cyclohexane:ethylacetate 7:3, 5 mL, R_{f}=0.13).

Colourless crystalline solid, Mp. 75° C., yield 46 mg (34%).

C₂₂H₂₉NO₂S (371.5).

HPLC: Method 1: %, R=min
Method 2:

MS (EI): m/z=371 [M⁺], 340 [M⁺-OCH₃], 252 [M⁺-(CH₂)₃Ph].

IR (Film): v (cm⁻¹)=3096 (C—H), 2922 (C—H), 1059 (C—O), 745 (C—H).

¹H-NMR (CDCl₃):

δ (ppm)=1.49-1.63 (m, 4H, N—CH₂CH₂CH₂CH₂.Ph), 1.78 (dd, J=13.7/2.7 Hz, 1H, N(CH₂CH₂)₂), 1.85 (td, J=12.4/4.5 Hz, 1H, N(CH₂CH₂)₂), 1.91 (dd, J=13.7/2.7 Hz, 1H, N(CH₂CH₂)₂), 2.04 (td, J=13.3/4.3 Hz, 1H, N(CH₂CH₂)₂), 2.26-2.45 (m, 4H(N(CH₂CH₂)₂)(N—CH2CH2CH2CH₂-Ph)), 2.58 (t, J=7.2 Hz, 2H, N—CH₂(CH₂)₃-Ph), 2.69-2.81 (m, 3H(N(CH₂CH₂)₂) (Th—CH₂CHOCH₃), 2.92 (dd, J=15.6/3.3 Hz, 1H, Th—CH₂CHOCH₃), 3.51 (s, 3H, Th—CH₂CHOCH₃), 4.81 (dd, J=7.2/3.3 Hz, 1H, Th—CH₂CHOCH₃), 6.72 (d, J=5.4 Hz, 1H, 4-H—Th), 7.02 (d, J=5.4 Hz, 1H, 5-H—Th), 7.10-7.23 (m, 5H, Ph-H).

### Example 13

### 1-(4-Flourbenzyl)-6′-Methoxy-6′,7′-dihydrospiro[piperidin-4,4′-thieno[3.2-c]pyran]

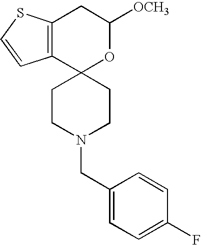

### Experimental Procedure

Example 2 (129 mg, 0.5 mmol) was dissolved in CH₃CN (20 mL), mixed with 4-fluorobenzylchloride (87 mg, 0.6 mmol) and K₂CO₃ (414 mg) and heated for 24 h under reflux. For purification K₂CO₃ was removed by a frit and the solution transferred into a separating funnel. After addition of saturated NaCl-solution (10 mL) it was extracted three times with Et₂O. The organic phases were pooled and dried over Na2SO4, then filtered and the solvent removed under vacuum. The crude product was purified using flash-chromatography (2 cm, cyclohexane:ethylacetate 7:3, 10 mL, R_{f}=0.32).

Colourless crystalline solid, Mp. 83° C., yield 83 mg (48%).

C₁₉H₂₂FNO₂S (347.4).

HPLC: Method 1: 98.8%, Rt=18.6 min
Method 2:

MS (EI): m/z=347 [M⁺], 316 [M⁺-OCH₃], 238 [M⁺-CH₂Ph-ₚF].

IR (Film): v (cm⁻¹)=3099 (C—H), 2812 (C—H), 1057 (C—O), 746 (C—H).

¹H-NMR (CDCl₃):

δ (ppm)=1.76 (dd, J=13.6/2.7 Hz, 1H, N(CH₂CH₂)₂), 1.83 (td, J=12.2/4.0 Hz, 1H, N(CH₂CH₂)₂), 1.89 (dd, J=13.6/2.5 Hz, 1H, N(CH₂CH₂)₂), 2.05 (td, J=13.2/4.5 Hz, 1H, N(CH₂CH₂)₂), 2.35 (td, J=12.6/3.3 Hz, 1H, N(CH₂CH₂)₂), 2.43 (td, J=12.6/2.6 Hz, 1H, N(CH₂CH₂)₂), 2.62-2.72 (m, 2H, N(CH₂CH₂)₂), 2.79 (dd, J=15.6/7.2 Hz, 1H, Th—CH₂CHOCH₃), 2.92 (dd, J=15.6/3.3 Hz, 1H, Th—CH₂CHOCH₃), 3.44 (d, J=13.3 Hz, 1H, N—CH₂Ph_{(4-F})), 3.47 (d, J=13.3 Hz, 1H, N—CH₂Ph_{(4-F)}), 3.49 (s, 3H, OCH₃), 4.80 (dd, J=7.2/3.3 Hz, 1H, Th—CH₂CHOCH₃), 6.72 (d, J=5.1 Hz, 1H, 4-H—Th), 6.91-6-98 (m, 2H, _{(4-F)}Ph-H), 7.02 (d, J=5.1 Hz, 1H, 5-H—Th), 7.23-7.29 (m, 2H, _{(4-F)}Ph-H).

### Example 14

### 6′-Methoxy-1-(4-methoxybenzyl)-6′,7′-dihydrospiro[piperidin-4,4′-thieno[3.2-c]pyran]

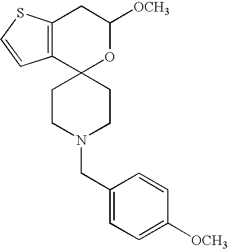

### Experimental Procedure

Example 2 (70 mg, 0.29 mmol) was dissolved in CH₂Cl₂ (5 mL), mixed with anisaldehyde (45 mg, 0.33 mmol) and NaBH(OAc)₃ (75 mg, 0.35 mmol) and stirred for 4 h at RT. After the end of the reaction a solution of NaHCO₃ (5 mL) was added and extracted three times with CH₂Cl₂ (5 mL). The organic phases were pooled and dried over Na2SO4, then filtered and the solvent removed under vacuum. The crude product (approx. 136 mg) was purified using flash-chromatography (1 cm, cyclohexane:ethylacetate 7:3, 5 mL, R_{f}=0.16).

Colourless crystalline solid, Mp. 91° C., yield 94 mg (91%).

C₂₀H₂₅NO₃S (359.5).

HPLC: Method 1: 96.8%, Rt=18.6 min
Method 2:

MS (EI): m/z=359 [M⁺], 297 [M⁺-OCH₃ (2×)], 238 [M⁺-CH₂Ph-ₚOCH₃].

IR (Film): v (cm⁻¹)=3096 (C—H), 2926 (C—H), 1060 (C—O), 719 (C—H).

¹H-NMR (CDCl₃):

δ (ppm)=1.75 (dd, J=13.7/2.5 Hz, 1H, N(CH₂CH₂)₂), 1.83 (td, J=12.5/4.5 Hz, 1H, N(CH₂CH₂)₂), 1.88 (dd, J=13.7/2.5 Hz, 1H, N(CH₂CH₂)₂), 2.03 (td, J=13.2/4.2 Hz, 1H, N(CH₂CH₂)₂), 2.34 (td, J=12.0/3.1 Hz, 1H, N(CH₂CH₂)₂), 2.43 (td, J=12.5/2.6 Hz, 1H, N(CH₂CH₂)₂), 2.65-2.75 (m, 2H, N(CH₂CH₂)₂), 2.77 (dd, J=15.6/7.1 Hz, 1H, Th—CH₂CHOCH₃), 2.92 (dd, J=15.6/3.3 Hz, 1H, Th—CH₂CHOCH₃), 3.43 (d, J=14.0 Hz, 1H, N—CH₂Ph_{(4-Ome)}), 3.47 (d, J=14.0 Hz, 1H, N—CH₂Ph_{(4-OMe)}), 3.49 (s, 3H, Th—CH₂CHOCH₃), 3.74 (s, 3H, Ph-OCH₃), 4.80 (dd, J=7.1/3.3 Hz, 1H, Th—CH₂CHOCH₃), 6.72 (d, J=5.5 Hz, 1H, 4-H—Th), 6.80 (d, J=8.4 Hz, 2H, _{(4-OMe)}Ph-H), 7.02 (d, J=5.1 Hz, 1H, 5-H—Th), 7.20 (d, J=8.4 Hz, 2H, _{(4-OMe)}Ph-H).

### Example 15

### 6′-Methoxy-1-(thien-2-ylmethyl)-6′,7′-dihydrospiro[piperidin-4,4′-thieno[3.2-c]pyran]

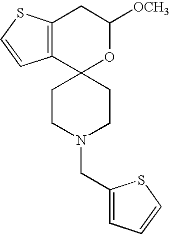

### Experimental Procedure

Example 2 (58 mg, 0.24 mmol) was dissolved in CH₂Cl₂ (5 mL), mixed with thiophene-2-carbaldehyde (31 mg, 0.28 mmol) and NaBH(OAc)₃ (80 mg, 0.38 mmol) and stirred for 2 h at RT. After the end of the reaction a saturated solution of NaHCO₃ (5 mL) was added and it was extracted three times with CH₂Cl₂ (5 mL). The organic phases were pooled and dried over Na2SO4, then filtered and the solvent removed under vacuum. The crude product (approx. 100 mg) was purified using flash-chromatography (1 cm, cyclohexane:ethylacetate 7:3, 5 mL, R_{f}=0.34).

Colourless crystalline solid, Mp. 73° C., yield 69 mg (82%).

C₁₇H₂₁NO₂S₂ (335.5).

HPLC: Method 1: 98.3%, Rt=17.5 min
Method 2:

MS (EI): m/z=335 [M⁺], 304 [M⁺-OCH₃], 238 [M⁺-CH₂Th], 97 [⁺CH₂Th].

IR (Film): v (cm⁻¹)=3100 (C—H), 2809 (C—H), 1058 (C—O), 749 (C—H).

¹H-NMR (CDCl₃):

δ (ppm)=1.77 (dd, J=13.6/2.7 Hz, 1H, N(CH₂CH₂)₂), 1.84 (td, J=12.2/4.3 Hz, 1H, N(CH₂CH₂)₂), 1.91 (dd, J=14.0/2.8 Hz, 1H, N(CH₂CH₂)₂), 2.05 (td, J=13.2/4.5 Hz, 1H, N(CH₂CH₂)₂), 2.42 (td, J=11.8/3.4 Hz, 1H, N(CH₂CH₂)₂), 2.51 (td, J=12.6/2.6 Hz, 1H, N(CH₂CH₂)₂), 2.73-2.81 (m, 3H(N(CH₂CH₂)₂) (Thₐ—CH₂CHOCH₃), 2.91 (dd, J=15.6/3.1 Hz, 1H, Thₐ—CH₂CHOCH₃), 3.48 (s, 3H, Thₐ CH₂CHOCH₃), 3.72 (d, J=13.7 Hz, 1H, N—CH₂—Th_{b}) 3.76 (d, J=13.7 Hz, 1H, N—CH₂—Th_{b}), 4.81 (dd, J=7.8/3.1 Hz, 1H, Thₐ—CH₂CHOCH₃), 6.73 (d, J=5.1 Hz, 1H, 4-H—Thₐ), 6.88 (m, 2H (3-H—Th_{b}) (4-H—Th_{b})), 7.02 (d, J=5.5 Hz, 1H, 5-H—Thₐ), 7.17 (dd, J=4.7/1.6 Hz, 1H, 5-H—Th_{b}).

### Example I

### 1-(Cyclohexylmethyl)piperidin-4-one

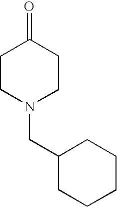

### Experimental Procedure

Piperidin-4-one hydrochloride monohydrate (0.7 g, 4.55 mmol) was dissolved in CH₃CN:H₂O 1:1 (15 mL), mixed with cyclohexylmethylbromide (0.96 mg, 5.47 mmol) and K₂CO₃ (3.8 g) and heated for 34 h under reflux. For purification it was extracted three times with CH₂Cl₂ (10 mL) and the organic phase dried over Na₂SO₄. Following filtration the solvent was removed under vacuum and the crude product purified by chromatography (3 cm, cyclohexane:ethylacetate 7:3, 15 mL, R_{f}=0.40).

Colourless oil, yield 656 mg (74%).

C₁₂H₂₁NO (195.1).

MS (EI): m/z=195 [M⁺], 112 [M⁺-cHex].

IR (Film): v (cm⁻¹)=2919 (C—H), 1718 (C═O), 758 (C—H).

¹H-NMR (CDCl₃):

δ (ppm)=0.78-0.88 (m, 2H, cHex-H), 1.05-1.25 (m, 3H, cHex-H), 1.38-1.43 (m, 1H, cHex-H), 1.58-1.69 (m, 3H, cHex-H), 1.61-1.68 (m, 2H, cHex-H), 2.17 (d, J=5.4 Hz, 2H, N—CH₂-cHex), 2.38 (t, J=4.5 Hz, 4H, N(CH₂CH₂)₂), 2.63 (t, J=4.5 Hz, 4H, N(CH₂CH₂)₂).

### Example J

### 1-(3-Methylbutyl)piperidin-4-on

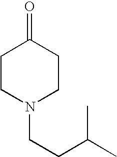

### Experimental Procedure

Piperidin-4-on hydrochlorid monohydrate (1.5 g, 9.76 mmol) was dissolved in CH₃CN:H₂O 1:1 (50 mL), mixed with 1-bromo-3-methylbutane (1.77 g, 11.7 mmol) and K₂CO₃ (8.1 g) and heated for 18 h under reflux. For purification it was extracted three times with CH₂Cl₂ (10 mL) and the organic phases dried over Na₂SO₄. Following filtration the solvent was removed under vacuum and the crude product purified by flash-chromatography (3 cm, cyclohexane:ethylacetate 7:3, 30 mL, R_{f}=0.34).

Colourless oil, yield 1.21 g (73%).

C₁₀H₁₉NO (169.3).

MS (EI): m/z=169 [M⁺], 112 [M⁺-CH₂CH(CH₃)₂].

IR (Film): v (cm⁻¹)=2953 (C—H), 1718 (C═O), 756 (C—H).

¹H-NMR (CDCl₃):

δ (ppm)=0.92 (d, J=5.1 Hz, 6H, N—CH₂CH₂CH(CH₃)₂), 1.38-1.44 (m, 1H, CH₂CH₂CH(CH₃)₂), 1.59-1.68 (m, 2H, N—CH₂CH₂CH(CH₃)₂), 2.45-2.52 (m, 6H, (N(CH₂CH₂)₂)(N—CH₂CH₂CH(CH₃)₂)), 2.86 (t, J=4.5 Hz, 4H, N(CH₂CH₂)₂).

### Example 16

### 1-Benzylspiro[piperidin-4,4′-thieno[3,2-c]pyran]

### Example 17

### 1-Benzyl-6′,7′-dihydrospiro[piperidin-4,4′-thieno[3,2-c]pyran]-6′-carbonitrile

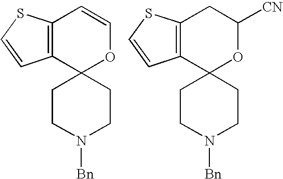

### Experimental Procedure

Example 3 (329 mg, 1 mmol) was dissolved in CH₂Cl₂ (20 mL) and cooled to −20° C. under N₂. Then TMS-CN (1 mL, 8 mmol) and BF₃.Et₂O (0.2 mL, 1.6 mmol) were added consecutively through a septum and stirred for 30 min at −20° C., and subsequently for 1 h at 0° C. in an ice bath. For purification MeOH (3 mL) and 2M NaOH (3 mL) were added (pH>9) and then extracted three times with CH₂Cl₂ (5 mL). The organic phases were pooled and dried over Na2SO4, then filtered and the solvent removed under vacuum. The crude product was purified using flash-chromatography (2 cm, cyclohexane:ethylacetate 7:3, 5 mL, R_{f}(11)=0.42, R_{f}(10)=0.34).

### Example 16

Colourless oil, yield 116 mg (38%).

C₁₈H₁₉NOS (297.4).

HPLC: Method 1: 97.0%, R=18.8 min
Method 2:

MS (EI): m/z=297 [M⁺], 206 [M⁺-CH₂Ph], 91 [⁺CH₂Ph].

IR (Film): v (cm⁻¹)=3024 (C—H), 2922 (C—H), 1598 (C═C), 1046 (C—O), 696 (C—H).

¹H-NMR (CDCl₃):

δ (ppm)=1.84 (td, J=13.4/4.6 Hz, 2H, N(CH₂CH₂)₂), 2.16 (dd, J=14.3/2.1 Hz, 2H, N(CH₂CH₂)₂), 2.37 (td, J=12.5/2.4 Hz, 2H, N(CH₂CH₂)₂), 2.68 (m, 2H, N(CH₂CH₂)₂), 3.50 (s, 2H, N—CH₂-Ph), 5.71 (d, J=5.9 Hz, 1H, Th—CH═CH), 6.38 (d, J=5.9 Hz, 1H, Th—CH═CH), 6.71 (d, J=5.1 Hz, 1H, 4-H—Th), 6.97 (d, J=5.1 Hz, 1H, 5-H—Th), 7.19-7.31 (m, 5H, Ph-H).

### Example 17

Colourless crystalline solid, Mp. 123° C., yield 178 mg (55%).

C₁₉H₂₀N₂OS (324.4).

HPLC: Method 1: 98.7%, Rt=18.4 min
Method 2:

MS (EI): m/z=324 [M⁺], 298 [M⁺-CN], 233 [M⁺-CH₂PH], 91 [⁺CH₂Ph].

IR (Film): v (cm⁻¹)=3090 (C—H), 2824 (C—H), 2243 (C≡N), 1067 (C—O), 696 (C—H).

¹H-NMR (CDCl₃):

δ (ppm)=1.77 (dd, J=13.8/2.7 Hz, 1H, N(CH₂CH₂)₂), 1.80-1.89 (m, 2H, N(CH₂CH₂)₂), 2.03 (td, J=13.2/4.5 Hz, 1H, N(CH₂CH₂)₂), 2.33 (td, J=11.8/3.1 Hz, 1H, N(CH₂CH₂)₂), 2.43 (td, J=12.6/2.7 Hz, 1H, N(CH₂CH₂)₂), 2.64-2.75 (m, 2H, N(CH₂CH₂)₂), 3.04 (dd, J=15.6/3.9 Hz, 1H, Th—CH₂CH), 3.16 (dd, J=15.6/9.0 Hz, 1H, Th—CH₂CH), 3.48 (d, J=13.0 Hz, 1H, N—CH₂-Ph), 3.53 (d, J=13.0 Hz, 1H, N—CH₂-Ph), 4.65 (dd, J=9.0/3.3 Hz, 1H, Th—CH₂CH), 6.75 (d, J=5.1 Hz, 1H, 4-H—Th), 7.09 (d, J=5.1 Hz, 1H, 5-H—Th), 7.19-7.31 (m, 5H, Ph-H).

¹³C-NMR (CDCl₃):

δ (ppm)=30.2 (1C, N(CH₂CH₂)₂), 35.6 (1C, N(CH₂CH₂)₂), 37.8 (1C, Th—CH₂CH), 49.0 (2C, N(CH₂CH₂)₂), 58.6 (1C, Th—CH₂CH), 63.6 (1C, N—CH₂-Ph), 76.4 (1C, Th—C—O), 118.6 (1C, CN), 124.2 (1C, Th—CH), 124.4 (1C, Th—CH), 127.3 (1C, Ph-CH), 128.5 (2C, Ph-CH), 128.8 (1C, Ph-C), 129.5 (2C, Ph-CH), 138.7 (1C, Th—C), 140.5 (1C, Th—C).

### Example 20

### 1-Benzyl-6′,7′-dihydrospiro[piperidin-4,4′-thieno[3,2-c]pyran]-6′-ol

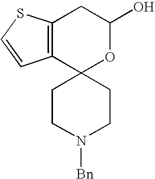

### Experimental Procedure

Example 3 (330 mg, 1.0 mmol) was dissolved in CH₃CN (7 mL), mixed with 2M HCl (3 mL) and heated for 1 h under reflux. Following that it was alkalised with 2M NaOH (pH>9) and extracted with Et₂O. For a better separation of the phases a few ml of a saturated solution of NaCl was added. The organic phases were pooled and dried over Na₂SO₄. Then it was filtered and the solvent removed under vacuum. The purification of the crude product was achieved by re-crystallization from Et₂O.

Colourless Solid, Mp. 184° C., yield: 163 g (68%).

C₁₈H₂₁NO₂S (315.4).

MS (EI): m/z=

IR (Film): v (cm⁻¹)=3100 (C—H), 1038 (C—O), 693 (C—H).

¹H-NMR (CDCl₃):

δ (ppm)=1.76 (dd, J=13.6/2.3 Hz, 1H, N(CH₂CH₂)₂), 1.80-1.89 (m, 2H, N(CH₂CH₂)₂), 2.04 (td, J=13.2/4.4 Hz, 1H, N(CH₂CH₂)₂), 2.37 (td, J=11.7/4.5 Hz, 1H, N(CH₂CH₂)₂), 2.43 (td, J=11.7/2.5 Hz, 1H, N(CH₂CH₂)₂), 2.62-2.72 (m, 2H, N(CH₂CH₂)₂), 2.74 (dd, J=15.5/7.3 Hz, Th—CH₂CH), 2.91 (s, 1H, OH), 3.00 (dd, J=15.5/3.1 Hz, 1H, Th—CH₂CH), 3.48 (d, J=14.2 Hz, 1H, N—CH₂Ph), 3.51 (d, J=14.2 Hz, 1H, N—CH₂Ph), 5.25 (dd, J=7.3/3.1 Hz, 1H, Th—CH₂CH), 6.74 (d, J=5.2 Hz, 1H, 4-H—Th), 7.04 (d, J=5.2 Hz, 1H, 5-H—Th), 7.16-7.29 (m, 5H, Ph-H).

### Example 18

### {1-Benzyl-6′,7′-dihyrospiro[piperidin-4,4′-thieno[3,2-c]pyran]-6′-yl}acetonitril

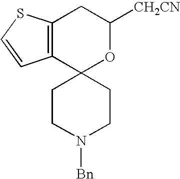

### Experimental Procedure

Example 20 (91 mg, 0.29 mmol) was dissolved in THF (5 mL), mixed with cyanomethylentriphenyl-phosphorane (126 mg, 0.42 mmol) and Cs₂CO₃ (100 mg, 0.3 mmol) and heated for 3.5 h under reflux. Subsequently H₂O (10 mL) was added and a few times extracted with CH₂Cl₂. The pooled organic phases were dried over Na₂SO₄ and then filtered. After removal of the solvent under vacuum the crude product was purified using flash-chromatography (1.5 cm, cyclohexane:ethylacetate 7:3, 10 mL, R_{f}=0.23).

Colourless crystalline solid, Mp. 133° C., yield 92 mg (94%).

C₂₀H₂₂N₂OS (338.5).

HPLC: Method 1: 98.4%, Rt=18.3 min
Method 2:

MS (EI): m/z=338 [M⁺], 298 [M⁺-CH₂CN], 247 [M⁺-CH₂Ph].

IR (Film): v (cm⁻¹)=3029 (C—H), 2923 (C—H), 2251 (C≡N), 1061 (C—O), 697 (C—H).

¹H-NMR (CDCl₃):

δ (ppm)=1.62 (dd, J=13.5/2.6 Hz, 1H, N(CH₂CH₂)₂), 1.79 (td, J=12.6/4.4 Hz, 1H, N(CH₂CH₂)₂), 1.91 (dd, J=14.3/2.7 Hz, 1H, N(CH₂CH₂)₂), 2.06 (td, J=13.1/4.6 Hz, 1H, N(CH₂CH₂)₂), 2.38-2.46 (m, 2H, N(CH₂CH₂)₂), 2.62-2.70 (m, 5H, (Th—CH₂CHCH₂CN)(N(CH₂CH₂)₂)), 2.80 (dd, J=15.6/3.1 Hz, 1H, Th—CH₂CHCH₂CN), 3.50 (s, 2H, N—CH₂Ph), 3.98-4.06 (m, 1H, Th—CH₂CHCH₂CN), 6.74 (d, J=5.1 Hz, 1H, 4-H—Th), 7.04 (d, J=5.1 Hz, 1H, 5-H—Th), 7.18-7.32 (m, 5H, Ph-H).

### Example 19

### 1-Benzyl-6′,7′-dihydrospiro[piperidin-4,4′-thieno[3,2-c]pyran]

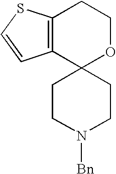

### Experimental Procedure

Unsaturated example 16 (60 mg, 0.2 mmol) was dissolved in a special pressure resistant flask in MeOH (8 mL) and mixed with Pd/C (15 mg, 20% (m/m)). The solution was shaken for 24 h under a pressure of 4.5 bar H₂ in an hydr Hydrogenation apparatus. After the end of the reaction Pd/C was filtered off and the solvent removed under vacuum. The crude product was purified using flash-chromatography (0.7 cm, cyclohexane:ethylacetate 9:1, 5 mL, R_{f}=0.20).

Colourless oil, yield 53.8 mg (90%).

C₁₈H₂₁NOS (299.4).

HPLC: Method 1: 97.2%, R=18.3 min
Method 2:

MS (EI): m/z=299 [M⁺], 208 [M⁺-CH₂PH], 91 [⁺CH₂Ph].

IR (Film): v (cm⁻¹)=3026 (C—H), 2922 (C—H), 1073 (C—O), 697 (C—H).

¹H-NMR (CDCl₃):

δ (ppm)=1.77 (dd, J=14.4/2.3 Hz, 2H, N(CH₂CH₂)₂), 1.91 (td, J=13.4/4.5 Hz, 2H, N(CH₂CH₂)₂), 2.33 (td, J=12.6/2.5 Hz, 2H, N(CH₂CH₂)₂), 2.64-2.67 (m, 2H, N(CH₂CH₂)₂), 2.75 (t, J=5.4 Hz, 2H, Th—CH₂CH₂), 3.49 (s, 2H, N—CH₂Ph), 3.85 (t, J=5.4 Hz, 2H, Th—CH₂CH₂), 6.74 (d, J=5.5 Hz, 1H, 4-H—Th), 6.98 (d, J=5.5 Hz, 1H, 5-H—Th), 7.19-7.30 (m, 5H, Ph-H).

### Example 21

### 1-(Cyclohexylmethyl)spiro[piperidine-4,4′ thieno[3,2-c]pyrano] and

### Example 22

### 1-(Cyclohexylmethyl)-6′,7′-dihydrospiro[piperidine-4,4′-thieno[3,2-c]pyrano]-6′-carbonitrile

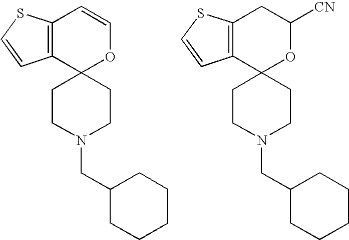

Example 5 (330 mg, 0.98 mmol) was dissolved in CH₂Cl₂ (10 mL) and cooled under N₂ to −20° C. Following that TMS-CN (1 mL, 7.8 mmol) and BF₃.Et₂O (175 μL, 1.4 mmol) were added one after the other through a septum and the mixture stirred for 30 min at −20° C. For purification 2 M NaOH (3 mL) were added (pH>9) and it was extracted with CH₂Cl₂ (3×5 mL). The organic phases were pooled and dried over Na₂SO₄. Then it was filtered and the solvent removed under vacuum. Both products were separated from one another using flash-chromatography (3 cm, cHex:EA 8:2, 10 mL, R_{f}(EX 21)=0.56, R_{f}(EX 22)=0.30).

EX 21: Colourless Solid, MP 75° C., Yield 91 mg (30%).

HPLC: 96.7%, t_{R}=19.8 min.

C₁₈H₂₅NOS (303.5)

MS (EI): m/z=303 [M⁺], 220 [M⁺-cHex].

IR (Film): v (cm⁻¹)=3043 (C—H), 2926 (C—H), 1592 (C═C), 1047 (C—O), 714 (C—H).

¹H-NMR (CDCl₃):

δ (ppm)=0.84-0.95 (m, 2H, cHex-H), 1.13-1.30 (m, 3H, cHex-H), 1.45-1.55 (m, 1H, NCH₂—CH(CH₂)₅), 1.63-1.74 (m, 3H, cHex-H), 1.75-1.84 (m, 2H, cHex-H), 1.89 (td, J=13.6/4.5 Hz, 2H, N(CH₂CH₂)₂), 2.17 (d, J=7.2 Hz, 2H, NCH₂-cHex), 2.19 (dd, J=14.4/2.8 Hz, 2H, N(CH₂CH₂)₂), 2.31 (td, J=12.8/2.8 Hz, 2H, N(CH₂CH₂)₂), 2.66-2.72 (m, 2H, N(CH₂CH₂)₂), 5.76 (d, J=6.4 Hz, 1H, ThCH═CH), 6.44 (d, J=6.4 Hz, 1H, ThCH═CH), 6.77 (d, J=5.2 Hz, 1H, 3′-H—Th), 7.03 (d, J=5.2 Hz, 1H, 2′-H—Th).

EX 22: Colourless Solid, MP 111° C., Yield 139 mg (43%).

HPLC: 95.9%, ambient temperature=19.8 min.

C₁₉H₂₆N₂OS (330.5).

MS (EI): m/z=330 [M⁺], 247 [M⁺-cHex].

IR (Film): v (cm⁻¹)=2920 (C—H), 1072 (C—O), 647 (C—H).

No band for CN is detectable.

¹H-NMR (CDCl₃):

δ (ppm)=0.84-0.95 (m, 2H, cHex-H), 1.13-1.29 (m, 3H, cHex-H), 1.45-1.55 (m, 1H, NCH₂—CH(CH₂)₅), 1.64-1.74 (m, 3H, cHex-H), 1.75-1.82 (m, 2H, cHex-H), 1.83-1.94 (m, 3H, N(CH₂CH₂)₂), 2.08 (td, J=13.6/4.8 Hz, 1H, N(CH₂CH₂)₂), 2.18 (d, J=7.2 Hz, 2H, NCH₂-cHex), 2.28 (td, J=11.6/3.2 Hz, 1H, N(CH₂CH₂)₂), 2.33 (td, J=11.6/3.2 Hz, 1H, N(CH₂CH₂)₂), 2.65-2.74 (m, 2H, N(CH₂CH₂)₂), 3.09 (dd, J=15.6/4.0 Hz, 1H, ThCH₂CH), 3.21 (dd, J=15.6/8.8 Hz, 1H, ThCH₂CH), 4.70 (dd, J=8.8/4.0 Hz, 1H, ThCH₂CH), 6.81 (d, J=5.2 Hz, 1H, 3′-H—Th), 7.15 (d, J=5.2 Hz, 1H, 2′-H—Th).

¹³C-NMR (CDCl₃):

δ (ppm)=26.0 (2C, cHex-C), 26.6 (1C, cHex-C), 26.7 (1C, cHex-C), 29.8 (1C, ThCH₂CH), 31.9 (2C, cHex-C), 35.1 (1C, N(CH₂CH₂)₂), 37.3 (1C, N(CH₂CH₂)₂), 49.0 (1C, N(CH₂CH₂)₂), 49.1 (1C, N(CH₂CH₂)₂), 58.0 (1C, ThCH₂CH), 65.7 (1C, NCH₂cHex), 76.1 (1C, ThCO), 118.2 (1C, CN), 123.7 (1C, 2′-C—Th), 124.0 (1C, 3′-C—Th), 128.3 (1C, 7a′-C—Th), 140.3 (1C, 3a′-C—Th).

### Example 23

### 1-(1-Benzyl-6′,7′-dihydrospiro[piperidine-4,4′-thieno[3.2-c]pyrano]-6′-yl)acetone

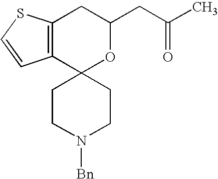

Example 20 (60 mg, 0.19 mmol) was dissolved in THF (4 mL), Acetylmethylentriphenyl-phosphorane (91 mg, 0.28 mmol) and Cs₂CO₃ (67 mg, 0.20 mmol) added and heated for 12 h under reflux. Following that H₂O (10 mL) was added and repeatedly extracted with CH₂Cl₂. The organic phases were pooled and dried over Na₂SO₄. After removing the solvent under vacuum the crude product was purified using flash-chromatography (1 cm, cHex:EA 7:3, 10 mL, R_{f}=0.25). Colourless Oil, Yield 45 mg (67%).

HPLC: 95.6%, t_{R}=18.7 min.

C₂₁H₂₅NO₂S (355.5).

MS (EI): m/z=355 [M⁺], 312 [M⁺-COCH₃], 264 [M⁺-CH₂Ph].

IR (Film): v (cm⁻¹)=3029 (C—H), 2811 (C—H), 1714 (C═O), 698 (C—H).

¹H-NMR (CDCl₃):

δ (ppm)=1.58 (dd, J=14.2/5.2 Hz, 1H, N(CH₂CH₂)₂), 1.73 (td, J=12.9/4.4 Hz, 1H, N(CH₂CH₂)₂), 1.94 (dd, J=14.2/5.2 Hz, 1H, N(CH₂CH₂)₂), 2.04 (td, J=13.2/4.2 Hz, 1H, N(CH₂CH₂)₂), 2.17 (td, J=11.8/2.4 Hz, 1H, N(CH₂CH₂)₂), 2.18 (s, 3H, CH₃), 2.30 (td, J=11.8/2.4 Hz, 1H, N(CH₂CH₂)₂), 2.49 (dd, J=15.0/4.0 Hz, 1H, ThCH₂CHCH₂), 2.55 (dd, J=15.6/10.2 Hz, 1H, ThCH₂CHCH₂), 2.58-2.67 (m, 2H, N(CH₂CH₂)₂), 2.70 (dd, J=15.6/3.1 Hz, 1H, ThCH₂CHCH₂) 2.82 (dd, J=15.0/8.5 Hz, 1H, ThCH₂CHCH₂), 3.45 (d, J=13.0 Hz, 1H, NCH₂Ph), 3.48 (d, J=13.0 Hz, 1H, NCH₂Ph), 4.18 (m, 1H, ThCH₂CHCH₂), 6.72 (d, J=5.2 Hz, 1H, 3′-H—Th), 6.99 (d, J=5.2 Hz, 1H, 2′-H—Th), 7.17-7.24 (m, 5H, Ph-H).

### Example 24

### Methyl-2-(1-benzyl-6′,7′-dihwospiro[piperidin-4,4′-thieno[3,2-c]pyrano]-6′-yl)-acetate

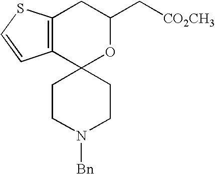

Example 20 (75 mg, 0.23 mmol) was dissolved in THF (6 mL), methoxycarbonyl-methylentriphenylphosphorane (130 mg, 0.38 mmol) and Cs₂CO₃ (83 mg, 0.25 mmol) added and heated for 6 h under reflux. Following that a solution of NaCl (10 mL) was added and it was repeatedly extracted with CH₂Cl₂. The organic phases were pooled and dried over Na₂SO₄. After removing the solvent under vacuum the crude product was purified using flash-chromatography (3 cm, cHex:EA 7:3, 30 mL, R_{f}=0.18). Colourless Solid, MP 65° C., Yield 62 mg (70%).

HPLC: 98.8%, t_{R}=19.4 min.

C₂₁H₂₅NO₃S (371.5).

MS (EI): m/z=371 [M⁺], 298 [M⁺-CH₂CO₂CH₃], 280 [M⁺-CH₂Ph], 91 [M⁺-CH₂Ph].

IR (Film): v (cm⁻¹)=3028 (C—H), 2922 (C—H), 1738 (C═O), 698 (C—H).

¹H-NMR (CDCl₃):

δ (ppm)=1.66 (dd, J=13.5/2.8 Hz, 1H, N(CH₂CH₂)₂), 1.80 (td, J=13.0/4.5 Hz, 1H, N(CH₂CH₂)₂), 2.02 (dd, J=13.5/2.8 Hz, 1H, N(CH₂CH₂)₂), 2.10 (td, J=13.0/4.5 Hz, 1H, N(CH₂CH₂)₂), 2.25 (td, J=11.2/2.8 Hz, 1H, N(CH₂CH₂)₂), 2.38 (td, J=12.8/2.8 Hz, 1H, N(CH₂CH₂)₂), 2.61 (dd, J=14.8/4.8 Hz, 1H, ThCH₂CHCH₂), 2.65 (dd, J=15.6/10.4 Hz, 1H, ThCH₂CHCH₂), 2.68-2.73 (m, 2H, N(CH₂CH₂)₂), 2.70 (dd, J=14.8/8.8 Hz, 1H, ThCH₂CHCH₂), 2.79 (dd, J=15.6/3.2 Hz, 1H, ThCH₂CHCH₂), 3.51 (d, J=13.2 Hz, 1H, NCH₂Ph), 3.54 (d, J=13.2 Hz, 1H, NCH₂Ph), 3.72 (s, 3H, CO₂CH₃), 4.21-4.29 (m, 1H, ThCH₂CHCH₂), 6.80 (d, J=5.2 Hz, 1H, 3′-H—Th), 7.06 (d, J=5.2 Hz, 1H, 2′-H—Th), 7.22-7.32 (m, 5H, Ph-H).

### Example 25

### Ethyl-2-(1-benzyl-6′,7′-dihydrospiro[piperidine-4,4′-thieno[3,2-c]pyrano]-6′-yl)acetate

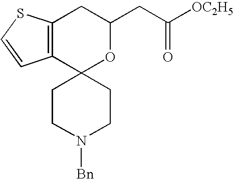

Example 20 (60 mg, 0.19 mmol) was dissolved in THF (3 mL), Ethoxycarbonyl-methylentriphenylphosphorane (129 mg, 0.42 mmol) and KO^{t}Bu (22 mg, 0.19 mmol) added and heated for 10.5 h under reflux. Following that H₂O (10 mL) was added and it was repeatedly extracted with CH₂Cl₂. The organic phases were pooled and dried over Na₂SO₄. After removing the solvent under vacuum the crude product was purified using flash-chromatography (1 cm, cHex:EA 7:3, 10 mL, R_{f}=0.18). Colourless Oil, Yield 34 mg (47%).

HPLC: 97.7%, t_{R}=20.4 min.

C₂₂H₂₇NO₃S (385.5).

MS (EI): m/z=385 [M⁺], 298 [M⁺-CH₂CO₂C₂H₅], 294 [M⁺-CH₂Ph].

IR (Film): v (cm⁻¹)=3024 (C—H), 2813 (C—H), 1732 (C═O), 698 (C—H).

¹H-NMR (CDCl₃):

δ (ppm)=1.31 (t, J=7.2 Hz, 3H, CO₂CH₂CH₃), 1.66 (dd, J=13.5/2.7 Hz, 1H, N(CH₂CH₂)₂), 1.81 (td, J=13.8/4.2 Hz, 1H, N(CH₂CH₂)₂), 2.03 (dd, J=14.0/2.7 Hz, 1H, N(CH₂CH₂)₂), 2.11 (td, J=14.2/2.7 Hz, 1H, N(CH₂CH₂)₂), 2.28 (td, J=13.2/2.2 Hz, 1H, N(CH₂CH₂)₂), 2.39 (td, J=13.0/2.3 Hz, 1H, N(CH₂CH₂)₂), 2.59 (dd, J=15.2/4.8 Hz, 1H, ThCH₂CHCH₂), 2.65-2.77 (m, 4H(N(CH₂CH₂)₂) (ThCH₂CHCH₂)), 2.79 (dd, J=15.6/3.2 Hz, 1H, ThCH₂CHCH₂), 3.52 (s, 2H, NCH₂Ph), 4.12-4.24 (m, 1H, ThCH₂CHCH₂), 4.18 (q, J=7.2 Hz, 2H, CO₂CH₂CH₃), 6.80 (d, J=5.2 Hz, 1H, 3′-H—Th), 7.07 (d, J=5.2 Hz, 1H, 2′-H—Th), 7.18-7.25 (m, 5H, Ph-H).

### Example 26

### 1-(Cyclohexylmethyl)-6′,7′-dihydrospiro[piperidine-4,4′-thieno[3,2-c]pyrano]

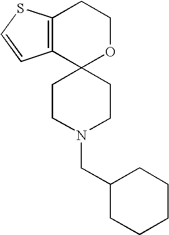

The unsaturated compound 21 (60 mg, 0.2 mmol) was dissolved in MeOH (8 mL) in a pressure resistant vessel and Pd/C (15 mg, 10% (m/m)) added. Under H₂-pressure of 4.5 bar the mixture was stirred for 6 h in a hydration apparatus. After the end of the reaction Pd/C was filtered off and the solvent removed in vacuum. The crude product was purified using flash-chromatography (0.7 cm, cHex:EA 9:1, 5 mL, R_{f}=0.2). Colourless Solid, MP 77° C., 31 mg (64%).

Analysis of elements: calc.: C, 70.77; H, 8.91; N, 4.59; found.: C, 70.85; H, 8.94; N, 4.59.

HPLC: 96.6° A), t_{R}=19.1 min.

C₁₈H₂₇NOS (305.5).

MS (EI): m/z=305 [M⁺], 222 [M⁺-cHex].

IR (Film): v (cm⁻¹)=3092 (C—H), 2925 (C—H), 1074 (C—O), 744 (C—H).

¹H-NMR (CDCl₃):

δ (ppm)=0.83-0.95 (m, 2H, cHex-H), 1.13-1.30 (m, 3H, cHex-H), 1.45-1.55 (m, 1H, NCH₂—CH(CH₂)₅), 1.63-1.74 (m, 3H, cHex-H), 1.75-1.83 (m, 2H, cHex-H), 1.83 (dd, J=14.4/2.8 Hz, 2H, N(CH₂CH₂)₂), 1.96 (td, J=13.6/4.5 Hz, 2H, N(CH₂CH₂)₂), 2.17 (d, J=7.2 Hz, 2H, NCH₂cHex), 2.27 (td, J=12.8/2.8 Hz, 2H, N(CH₂CH₂)₂), 2.65-2.71 (m, 2H, N(CH₂CH₂)₂), 2.82 (t, J=5.6 Hz, 2H, ThCH₂CH₂), 3.92 (t, J=5.6 Hz, 2H, ThCH₂CH₂), 6.81 (d, J=5.2 Hz, 1H, 3′-H —Th), 7.06 (d, J=5.2 Hz, 1H, 2′-H-TH).

### Example 27

### 2-(1-Benzyl-6′,7′-dihyrospiro[piperidine-4,4′-thieno[3,2-c]pyrano]-6′-yl)ethanol

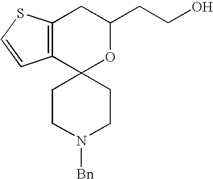

Example 30 (see below) (25 mg, 0.065 mmol) was dissolved in THF (2 mL) under N₂ and cooled to −20° C. Following careful addition of a solution of LiAlH₄ (1 M in THF, 140 μL, 0.13 mmol) it was stirred for 30 min at −20° Ct. Thereafter, the superfluous LiAlH₄ was destroyed by adding a saturated solution of NaCl. After repeated extraction with CH₂Cl₂ the organic phases were pooled, dried over Na₂SO₄ and filtered. After removing the solvent under vacuum the crude product was purified using flash-chromatography (0.8 cm, cHex:EA 2:8, 5 mL, R_{f}=0.18). Slightly yellowish resin, Yield 18.3 mg (80%).

HPLC: 98.0%, t_{R}=16.8 min.

C₂₀H₂₅NO₂S (343.5).

MS (EI): m/z=343 [M⁺], 298 [M⁺-(CH₂)₂OH], 252 [M⁺-CH₂Ph].

IR (Film): v (cm⁻¹)=3364 (O—H), 3028 (C—H), 2922 (C—H), 1056 (C—O), 697 (C—H).

¹H-NMR (CDCl₃):

δ (ppm)=1.70 (dd, J=13.6/2.8 Hz, 1H, N(CH₂CH₂)₂), 1.87 (td, J=14.4/4.4 Hz, 1H, N(CH₂CH₂)₂), 1.90-1.97 (m, 2H, CH₂CH₂OH), 2.03 (dd, J=14.4/2.8 Hz, 1H, N(CH₂CH₂)₂), 2.18 (td, J=13.2/4.8 Hz, 1H, N(CH₂CH₂)₂), 2.32-2.42 (m, 2H, N(CH₂CH₂)₂), 2.47 (s, 1H, OH), 2.71-2.74 (m, 2H, ThCH₂CH), 2.75-2.83 (m, 2H, N(CH₂CH₂)₂), 3.59 (s, 2H, NCH₂Ph), 3.88 (t, J=5.2 Hz, 2H, CH₂CH₂OH), 3.96-4.05 (m, 1 H, ThCH₂CH), 6.81 (d, J=5.2 Hz, 1H, 3′-H—Th), 7.07 (d, J=5.2 Hz, 1H, 2′-H—Th), 7.24-7.37 (m, 5H, Ph-H).

### Example 28

### 1-Benzylspiro[piperidine-4,4′-thieno[3.2-c]pyrano]-6′(7′H)-one

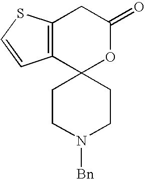

Example 20 (50 mg, 0.16 mmol) was dissolved in CH₂Cl₂ (4 mL) and N-Methylmorpholin-N-oxid (NMO) and N(iPr)₄RuO₄ (TPAP) added under N₂. After stirring for 2 h at ambient temperature a saturated solution of NaCl (5 mL) was added and extracted with CH₂Cl₂ (3×5 mL). The organic phase was dried over NaSO4 and filtered thereafter. After removing the solvent under vacuum the crude product was purified using flash-chromatography (0.7 cm, cHex:EA 1:1, 3 mL, R_{f}=0.35). Colourless crystalline Solid, MP 153° C., Yield 33 mg (66%).

HPLC: 99.6%, t_{R}=15.9 min.

C₁₈H₁₉NO₂S (313.4).

MS (EI): m/z=313 [M⁺], 222 [M⁺-CH₂Ph], 91 [⁺CH₂Ph].

IR (Film): v (cm⁻¹)=3097 (C—H), 2827 (C—H), 1722 (C═O), 696 (C—H).

¹H-NMR (CDCl₃):

δ (ppm)=1.88-1.94 (m, 2H, N(CH₂CH₂)₂), 2.21 (td, J=13.3/4.3 Hz, 2H, N(CH₂CH₂)₂), 2.61 (td, J=12.5/2.3 Hz, 2H, N(CH₂CH₂)₂), 2.78-2.82 (m, 2H, N(CH₂CH₂)₂), 3.60 (s, 2H, NCH₂Ph), 3.83 (s, 2H, ThCH₂), 6.88 (d, J=5.1 Hz, 1H, 3′-H—Th), 7.19 (d, J=5.1 Hz, 1H, 2′-H—Th), 7.22-7.33 (m, 5H, Ph-H).

### Example 29

### 1-Benzyl-6′,7′-dihyrospiro[piperidine-4,4′-thieno[3,2-c]pyrano]-6′-carboxamide

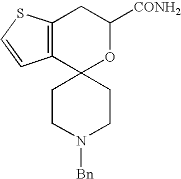

Example 17 (98 mg, 0.31 mmol) was dissolved in AcOH (3.5 mL) and cooled in ice to 2° C. Following that TiCl₄ (170 μL, 1.55 mmol) was slowly added under stirring. After 15 min H₂O (66 μL, 3.6 mmol) was added and further stirred 2 h at ambient temperature. For purification 2 M NaOH (20 mL) was added and it was repeatedly extracted with CH₂Cl₂. The organic phases were pooled and dried over Na₂SO₄ After removing the solvent under vacuum the crude product was purified using flash-chromatography (2 cm, cHex:EA 1.5:8.5, 10 mL, R_{f}=0.16). Colourless Solid, MP 63-65° C., Yield 60 mg (59%).

HPLC: 95.9%, t_{R}=15.7 min.

C₁₉H₂₂N₂O₂S (342.5).

MS (EI): m/z=342 [M⁺], 298 [M⁺-CONH₂], 251 [M⁺-CH₂Ph].

IR (Film): v (cm⁻¹)=3470 (N—H), 2807 (C—H), 1684 (C═O), 1072 (C—O), 698 (C—H).

¹H-NMR (CDCl₃):

δ (ppm)=1.75 (dd, J=13.6/2.7 Hz, 1H, N(CH₂CH₂)₂), 1.87 (td, J=12.4/4.4 Hz, 1H, N(CH₂CH₂)₂), 1.97 (dd, J=14.5/2.8 Hz, 1H, N(CH₂CH₂)₂), 2.18 (td, J=13.2/4.6 Hz, 1H, N(CH₂CH₂)₂), 2.28 (td, J=12.2/2.9 Hz, 1H, N(CH₂CH₂)₂), 2.39 (td, J=11.8/2.6 Hz, 1H, N(CH₂CH₂)₂), 2.73-2.78 (m, 1H, N(CH₂CH₂)₂), 2.80-2.86 (m, 1H, N(CH₂CH₂)₂), 2.82 (dd, J=16.0/11.1 Hz, 1H, ThCH₂CH), 3.24 (dd, J=16.0/3.6 Hz, 1H, ThCH₂CH), 3.57 (s, 2H, NCH₂Ph), 4.27 (dd, J=11.1/3.6 Hz, 1H, ThCH₂CH), 5.55 (s, 1H, NH₂), 6.68 (s, 1H, NH₂), 6.80 (d, 5.2 Hz, 1H, 3′-H—Th), 7.11 (d, J=5.2 Hz, 1H, 2′-H—Th), 7.22-7.34 (m, 5H, Ph-H).

### Example 30

### 1-Benzyl-6′,7′-dihyrospiro[piperidine-4,4′-thieno[3,2-c]pyrano]-6′-carbaldehyde

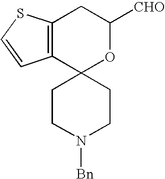

Example 22 (100 mg, 0.3 mmol) was dissolved in toluol (4 mL) and under N₂ cooled to −78° C. After slowly adding of a solution of diisobutylaluminiumhydride (1 M, 0.60 mL, 0.60 mmol) the mixture was stirred for 45 min at −78° C. and a saturated solution of NH₄Cl (5 mL) added. After heating to ambient temperature 2 M NaOH (3 mL) were added under strong stirring and precipitated Al(OH)₃ separated by a frit. Following that it was extracted with CH₂Cl₂ (3×5 mL) and the pooled organic phases dried over Na₂SO₄. After filtration the solvent was removed under vacuum and the crude product purified by flash-chromatography (1 cm, cHex:EA 1:1, 10 mL, R_{f}=0.21). Colourless, viscous oil, yield 36 mg (37%).

C₁₉H₂₁NO₂S (327.4).

MS (EI): m/z=327 [M⁺], 298 [M⁺-CHO], 236 [M⁺-CH₂Ph].

IR (Film): v (cm⁻¹)=2921 (C—H), 1737 (C═O), 1071 (C—O), 697 (C—H).

¹H-NMR (CDCl₃):

δ (ppm)=1.70 (dd, J=13.7/2.5 Hz, 1H, N(CH₂CH₂)₂), 1.82 (td, J=12.2/4.4 Hz, 1H, N(CH₂CH₂)₂), 1.90 (dd, J=13.4/2.7 Hz, 1H, N(CH₂CH₂)₂), 2.12 (td, J=13.2/4.6 Hz, 1H, N(CH₂CH₂)₂), 2.33 (td, J=11.8/3.1 Hz, 1H, N(CH₂CH₂)₂), 2.48 (td, J=11.4/2.5 Hz, 1H, N(CH₂CH₂)₂), 2.60-2.80 (m, 3H, (ThCH₂CH)(N(CH₂CH₂)₂)), 2.91 (dd, J=15.9/3.8 Hz, 1H, ThCH₂CH), 3.51 (s, 2H, NCH₂Ph)), 4.13 (dd, J=10.8/3.8 Hz, 1H, ThCH₂CH), 6.75 (d, J=5.2 Hz, 1H, 3′-H—Th), 7.04 (d, J=5.2 Hz, 1H, 2′-H—Th), 7.18-7.28 (m, 5H, Ph-H), 9.80 (s, 1H, CHO).

### Example 31

### (1-Benzyl-6′,7′-dihydrospiro[piperidine-4,4′-thieno[3,2-c]pyrano]-6′-yl)methanamine

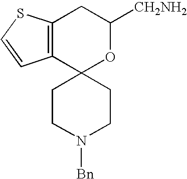

Example 22 (100 mg, 0.31 mmol) was dissolved in THF (4 mL) under N₂ and cooled to −10° C. After careful addition of LiAlH₄ (24 mg, 0.62 mmol) the mixture was stirred for 1 h at −10° C. Following that it was alkalised with 2 M NaOH and precipitated Al(OH)₃ removed through a frit. After repeated extraction of the filtrate with CH₂Cl₂ the organic phases were pooled, dried over Na₂SO₄ and filtered. After removing the solvent under vacuum the crude product was purified using flash-chromatography (1 cm, cHex:EA:MeOH 1:7:2, 10 mL, R_{f}=0.28). Slightly yellowish resin, Yield 57 mg (57) %).

HPLC: 95.1%, t_{R}=13.5 min

C₁₉H₂₄N₂OS (328.5).

MS (EI): m/z=328 [M⁺], 298 [M⁺-CH₂NH₂], 237 [M⁺-CH₂Ph].

IR (Film): v (cm⁻¹)=3328 (N—H), 3025 (C—H), 2919 (C—H), 1070 (C—O), 697 (C—H).

¹H-NMR (CDCl₃):

δ (ppm)=1.62 (dd, J=13.5/2.7 Hz, 1H, N(CH₂CH₂)₂), 1.77 (td, J=13.2/4.4 Hz, 1H, N(CH₂CH₂)₂), 1.95 (dd, J=14.4/2.7 Hz, 1H, N(CH₂CH₂)₂), 2.09 (td, J=13.1/4.5 Hz, 1H, N(CH₂CH₂)₂), 2.29 (td, J=12.6/2.6 Hz, 1H, N(CH₂CH₂)₂), 2.42 (td, J=12.6/2.6 Hz, 1H, N(CH₂CH₂)₂), 2.54 (dd, J=15.0/10.0 Hz, 1H, ThCH₂CHCH₂), 2.61 (dd, J=15.6/3.7 Hz, 1H, ThCH₂CHCH₂), 2.64-2.72 (m, 2H, N(CH₂CH₂)₂), 2.76-2.83 (m, 2H, ThCH₂CHCH₂), 3.50 (s, 2H, NCH₂Ph), 3.66-3.72 (m, 1H, ThCH₂CHCH₂), 6.74 (d, J=5.2 Hz, 1H, 3′-H—Th), 7.00 (d, J=5.2 Hz, 1H, 2′-H—Th), 7.18-7.27 (m, 5H, Ph-H). The signals of the 2 protons of the NH₂-group are not visible.

### Example 32

### Methyl-1-benzyl-6′,7′-dihyrospiro[piperidine-4,4′-thieno[3,2-c]pyrano]-6′-carboxylate

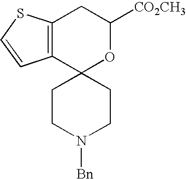

Example 22 (58 mg, 0.18 mmol) was dissolved in MeOH (3 mL), H₂SO₄ conc. (1 g) and 3 drops of H₂O were added and heated for 40 h under reflux. After finishing the reaction the mixture was neutralized with 2 M NaOH under cooling with ice (pH 7-8) and following addition of a saturated solution of NaCl repeatedly extracted with Et₂O. The organic phases were pooled and dried over Na₂SO₄. After removing the solvent under vacuum the crude product was purified using flash-chromatography (1 cm, cHex:EA 8:2, 5 mL, R_{f}=0.13). Colourless Oil, Yield 37 mg (56%).

HPLC: 96.9%, t_{R}=18.5 min.

C₂₀H₂₃NO₃S (357.5).

MS (EI): m/z=357 [M⁺], 298 [M⁺-CO₂CH₃], 266 [M⁺-CH₂Ph].

IR (Film): v (cm⁻¹)=3024 (C—H), 2923 (C—H), 1760 (C═O), 697 (C—H).

¹H-NMR (CDCl₃):

δ (ppm)=1.73 (dd, J=13.5/2.8 Hz, 1H, N(CH₂CH₂)₂), 1.78 (td, J=12.6/4.4 Hz, 1H, N(CH₂CH₂)₂), 1.90 (dd, J=14.5/2.8 Hz, 1H, N(CH₂CH₂)₂), 2.07 (td, J=13.1/4.6 Hz, 1H, N(CH₂CH₂)₂), 2.33 (td, J=12.5/2.8 Hz, 1H, N(CH₂CH₂)₂), 2.48 (td, J=12.7/2.6 Hz, 1H, N(CH₂CH₂)₂), 2.60-2.65 (m, 1H, N(CH₂CH₂)₂), 2.67-2.72 (m, 1H, N(CH₂CH₂)₂), 2.94 (dd, J=15.8/9.6 Hz, 1H, ThCH₂CH), 2.98 (dd, J=15.8/4.8 Hz, 1H, ThCH₂CH), 3.48 (d, J=13.0 Hz, 1H, NCH₂Ph), 3.52 (d, J=13.0 Hz, 1H, NCH₂Ph), 3.76 (s, 3H, CO₂CH₃), 4.32 (dd, J=9.6/4.8 Hz, 1H, ThCH₂CH), 6.74 (d, J=5.2 Hz, 1H, 3′-H—Th), 7.03 (dd, J=5.2 Hz, 1H, 2′-H—Th), 7.15-7.28 (m, 5H, Ph-H).

### Example 33

### Ethyl-1-benzyl-6′,7′-dihvrospiro[piperidine-4,4′-thieno[3,2-c]pyrano]-6′-carboxylate

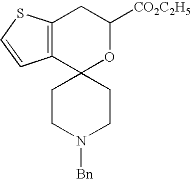

Example 22 (59 mg, 0.18 mmol) was dissolved in EtOH (3 mL), H₂SO₄ conc. (1 g) and 3 drops of H₂O added and heated for 18 h under reflux. After finishing the reaction the mixture was neutralized with 2 M NaOH under cooling with ice (pH 7-8) and following addition of saturated solution of NaCl repeatedly extracted with Et₂O. The organic phases were pooled and dried over Na₂SO₄. After removing the solvent under vacuum the crude product was purified using flash-chromatography (1 cm, cHex:EA 7:3, 5 mL, R_{f}=0.18). Colourless oil, yield 49 mg (71%).

HPLC: 98.6%, t_{R}=19.4 min.

C₂₁H₂₅NO₃S (371.5).

MS (EI): m/z=371 [M⁺], 298 [M⁺-CO₂C₂H₅], 280 [M⁺-CH₂Ph].

IR (Film): v (cm⁻¹)=3029 (C—H), 2924 (C—H), 1734 (C═O), 697 (C—H).

¹H-NMR (CDCl₃):

δ (ppm)=1.26 (t, J=7.2 Hz, 3H, CO₂CH₂CH₃), 1.72 (dd, J=13.5/2.7 Hz, 1H, N(CH₂CH₂)₂), 1.77 (td, J=12.6/4.4 Hz, 1H, N(CH₂CH₂)₂), 1.90 (dd, J=14.4/2.8 Hz, 1H, N(CH₂CH₂)₂), 2.07 (td, J=13.5/4.6 Hz, 1H, N(CH₂CH₂)₂), 2.34 (td, J=12.6/2.6 Hz, 1H, N(CH₂CH₂)₂), 2.49 (td, J=12.5/2.6 Hz, 1H N(CH₂CH₂)₂), 2.58-2.64 (m, 1H, N(CH₂CH₂)₂), 2.66-2.71 (m, 1H, N(CH₂CH₂)₂), 2.93 (dd, J=15.8/9.6 Hz, 1H, ThCH₂CH), 2.98 (dd, J=15.8/4.8 Hz, 1H, ThCH₂CH), 3.48 (d, J=13.0 Hz, 1H, NCH₂Ph), 3.52 (d, J=13.0 Hz, 1H, NCH₂Ph), 4.08-4.18 (m, 2H, CO₂CH₂CH₃), 4.29 (dd, J=9.6/4.8 Hz, 1H, ThCH₂CH), 6.74 (d, J=5.2 Hz, 1H, 3′-H—Th), 7.03 (dd, J=5.2 Hz, 1H, 2′-H—Th), 7.15-7.28 (m, 5H, Ph-H).

### Example 34

### (1-Benzyl-6′,7′-dihvrospiro[piperidine-4,4′-thieno[3,2-c]pyrano]-6′-yl)methanol

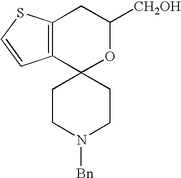

Example 30 (85 mg, 0.26 mmol) was dissolved in MeOH (3 mL) and under cooling with ice NaBH₄ (20 mg, 0.52 mmol) was added. After 15 min of stirring at 0° C. the mixture was stirred a further 45 min at ambient temperature. Following that a saturated solution of NaCl was added and the mixture was extracted 3 times with 5 mL CH₂Cl₂. The organic phases were pooled and dried over Na₂SO₄. After removing the solvent under vacuum the crude product was purified using flash-chromatography (1 cm, cHex:EA 7:3, 10 mL, R_{f}=0.08). Colourless solid, MP 108° C., Yield 60 mg (70

HPLC: 98.9%, t_{R}=16.5 min.

C₁₉H₂₃NO₂S (329.5).

MS (EI): m/z=329 [M⁺], 298 [M⁺-CH₂OH], 238 [M⁺-CH₂Ph].

IR (Film): v (cm⁻¹)=3428 (O—H), 3028 (C—H), 2849 (C—H), 1054 (C—O), 697 (C—H).

¹H-NMR (CDCl₃): δ (ppm)=1.69 (dd, J=13.6/2.8 Hz, 1H, N(CH₂CH₂)₂), 1.83 (td, J=12.8/4.4 Hz, 1H, N(CH₂CH₂)₂), 2.02 (dd, J=13.6/2.8 Hz, 1H, N(CH₂CH₂)₂), 2.15 (td, J=13.2/4.8 Hz, 1H, N(CH₂CH₂)₂), 2.30 (td, J=12.8/2.4 Hz, 1H, N(CH₂CH₂)₂), 2.44 (td, J=12.0/2.4 Hz, 1H, N(CH₂CH₂)₂), 2.64 (dd, J=16.0/4.0 Hz, 1H, ThCH₂), 2.67-2.78 (m, 3H(N(CH₂CH₂)₂) (ThCH₂)), 3.55 (s, 2H, NCH₂Ph), 3.70 (dd, J=11.0/7.3 Hz, 1H, ThCH₂CHCH₂), 3.78-3.84 (m, 1H, ThCH₂CHCH₂), 3.90-3.97 (m, 1H, ThCH₂CHCH₂), 6.81 (d, J=5.2 Hz, 1H, 3′-H—Th), 7.08 (d, J=5.2 Hz, 1H, 2′-H—Th), 7.23-7.34 (m, 5H, Ph-H). A signal for the proton of the OH-group was not detectable.

### Example 35

### (1-Benzyl-6′,7′-dihydrospiro[piperidine-4,4′-thieno[3,2-c]pyrano]-6′-yl)-N,N-dimethylmethanamine

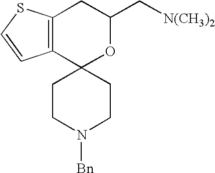

Example 30 (70 mg, 0.22 mmol) was dissolved in CH₂Cl₂ (2 mL), NaBH(OAc)₃ (70 mg, 0.34 mmol) and a solution of dimethylamine (2 M in THF, 120 μl, 0.24 mmol) added. After stirring for 2 h at ambient temperature a saturated solution of NaHCO₃ (3 mL) was added and the mixture was extracted repeatedly with CH₂Cl₂. The organic phases were pooled and dried over Na₂SO₄. After removing the solvent under vacuum the crude product was purified using flash-chromatography (1 cm, cHex:EA:MeOH 1:7:2, 10 mL, R_{f}=0.24). Colourless Oil, Yield 36 mg (46%).

HPLC: 97.8%, t_{R}=13.9 min.

C₂₁H₂₈N₂OS (356.5).

MS (EI): m/z=356 [M⁺], 298 [M⁺-CH₂N(CH₃)₂], 265 [M⁺-CH₂Ph].

IR (Film): v (cm⁻¹)=3029 (C—H), 2815 (C—H), 1068 (C—O), 697 (C—H).

¹H-NMR (CDCl₃):

δ (ppm)=1.62 (dd, J=13.6/2.8 Hz, 1H, N(CH₂CH₂)₂), 1.79 (td, J=12.8/4.8 Hz, 1H, N(CH₂CH₂)₂), 1.93 (dd, J=13.6/2.8 Hz, 1H, N(CH₂CH₂)₂), 2.09 (td, J=13.2/4.4 Hz, 1H, N(CH₂CH₂)₂), 2.29 (s, 6H, N(CH₃)₂), 2.34-2.47 (m, 2H, N(CH₂CH₂)₂), 2.52-2.58 (m, 3H, ThCH₂CHCH₂), 2.66-2.73 (m, 3H(N(CH₂CH₂)₂), (ThCH₂CHCH₂)), 3.55 (s, 2H, NCH₂Ph), 3.86-3.94 (m, 1H, ThCH₂CHCH₂), 6.75 (d, J=5.2 Hz, 1H, 3′-H—Th), 7.00 (d, J=5.2 Hz, 1H, 2′-H—Th), 7.18-7.25 (m, 5H, Ph-H).

### Example 36

### (1-Benzyl-6′,7′-dihydrospiro[piperidine-4,4′-thieno[3,2-c]pyrano]-6′-yl)-N-benzyl-methanamine

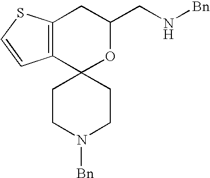

Example 34 (55 mg, 0.17 mmol) was dissolved in CH₂Cl₂ (3 mL), benzaldehyde (17.7 mg, 0.17 mmol) and NaBH(OAc)₃ (53 mg, 0.25 mmol) were added and the mixture stirred for 1 h at ambient temperature under N₂. After the reaction finished a saturated solution of NaHCO₃ (5 mL) was added and the mixture was extracted 3-times with CH₂Cl₂ (5 mL). The organic phases were pooled, dried over Na₂SO₄, filtered and the solvent removed under vacuum. The crude product was purified using flash-chromatography (1 cm, cHex:EA:MeOH 1.5:8:0.5, 3 mL, R_{f}=0.16). Colourless Oil, Yield 17.9 mg (25%).

HPLC: 96.0%, t_{R}=16.8 min.

C₂₆H₃₀N₂OS (418.6).

MS (EI): m/z=418 [M⁺], 327 [M⁺-CH₂Ph], 298 [M⁺-CH₂NHCH₂Ph].

IR (Film): v (cm⁻¹)=3331 (N—H), 3025 (C—H), 2920 (C—H), 696 (C—H).

¹H-NMR (CDCl₃):

δ (ppm)=1.75 (dd, J=14.4/2.8 Hz, 1H, N(CH₂CH₂)₂), 1.81 (td, J=14.4/4.4 Hz, 1H, N(CH₂CH₂)₂), 2.03 (dd, J=14.4/2.8 Hz, 1H, N(CH₂CH₂)₂), 2.14 (td, J=14.4/4.4 Hz, 1H, N(CH₂CH₂)₂), 2.31 (td, J=13.6/2.8 Hz, 1H, N(CH₂CH₂)₂), 2.44 (td, J=13.6/2.8 Hz, 1H, N(CH₂CH₂)₂), 2.63-2.67 (m, 2H, ThCH₂CHCH₂), 2.68-2.78 (m, 2H, N(CH₂CH₂)₂), 2.81 (dd, J=12.0/3.6 Hz, 1H, ThCH₂CH), 2.84 (dd, J=12.0/8.0 Hz, 1H, ThCH₂CH), 3.50 (d, J=12.8 Hz, 1H, NCH₂Ph), 3.55 (d, J=12.8 Hz, 1H, NCH₂Ph), 3.85 (d, J=13.6 Hz, 1H, NHCH₂Ph), 3.89 (d, J=13.6 Hz, 1H, NHCH₂Ph), 3.91-4.02 (m, 1H, ThCH₂CH), 6.79 (d, J=5.2 Hz, 1H, 3′-H—Th), 7.06 (dd, J=5.2 Hz, 1H, 2′-H—Th), 7.21-7.38 (m, 10H, Ph-H). A signal for the proton of the NH-group is not detected.

### Example K

### 4-Bromo-5-(2,2-dimethoxyethyl)thiophen-2-carbaldehyde

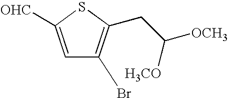

Example C (250 mg, 1 mmol) was dissolved under N₂ in THF (5 mL) and cooled to −40° C. A solution of LDA (2 M in THF, 890 μL, 1.79 mmol) was slowly added while stirring and the mixture stirred for a further 10 min. Thereafter, 1-Formylpiperidine (200 mg, 1.79 mmol), dissolved in THF (0.5 mL) was added. After 30 min the mixture was heated to ambient temperature, a saturated solution of NaCl (5 mL) added and extracted with CH₂Cl₂ (3×5 mL). The organic phase was dried over Na2SO4, filtered and the solvent removed under vacuum. The crude product was purified using flash-chromatography (3 cm, cHex:EA 9:1, 15 mL, R_{f}=0.18). Colourless Liquid, Yield 259 mg (93%).

HPLC: 99.7%, t_{R}=19.9 min.

C₉H₁₁BrO₃S (279.2).

MS (EI): m/z=280/278 [M⁺], 249/247 [M⁺-OCH₃].

IR (Film): v (cm⁻¹)=3083 (C—H), 2932 (C—H), 1668 (C═O), 1066 (C—O), 661 (C—H).

¹H-NMR (CDCl₃):

δ (ppm)=3.16 (d, J=5.6 Hz, 2H, ThCH₂), 3.40 (s, 6H, CH(OCH₃)₂), 4.58 (t, J=5.6 Hz, 1H, ThCH₂CH), 7.60 (s, 1H, 3-H—Th), 9.80 (s, 1H, ThCHO).

### Example L

### 4-Bromo-5-(2,2-dimethoxyethyl)thiophen-2-carbaldehydedimethylacetal

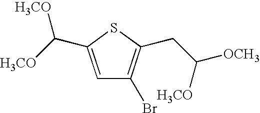

Example K (700 mg, 2.5 mmol) was dissolved in methanol (15 mL), Trimethylorthoformiate (3 mL) and p-Toluolsulfonic acid (24 mg, 125 μmol) added and heated for 3 h under reflux. For purification the mixture was made alkaline with 2 M NaOH (5 mL) and extracted with CH₂Cl₂ (3×10 mL). After drying the organic phase over Na₂SO₄ the mixture was filtered and the solvent removed under vacuum. The crude product was purified using flash-chromatography (3 cm, cHex:EA 9:1, 30 mL, R_{f}=0.41). Colourless Liquid, Yield 752 mg (92%).

HPLC: 99.6%, t_{R}=19.6 min.

C₁₁H₁₇BrO₄S (325.2).

MS (EI): m/z=326/324 [M⁺], 295/293 [M⁺-OCH₃].

IR (Film): v (cm⁻¹)=2932 (C—H), 2830 (C—H), 1049 (C—O).

¹H-NMR (CDCl₃):

δ (ppm)=3.07 (d, J=5.6 Hz, ThCH₂CH(OCH₃)₂), 3.35 (s, 6H, ThCH₂CH(OCH₃)₂), 3.38 (s, 6H, ThCH(OCH₃)₂), 4.54 (t, J=5.6 Hz, 1H, ThCH₂CH), 5.53 (s, 1H, ThCH(OCH₃)₂), 6.90 (s, 1H, 3-H—Th).

### Example M

### 2-(3-Bromo-5-chlorothiophene-2-yl)-acetaldehydedimethylacetal

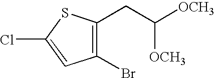

Example C (300 mg, 1.2 mmol) was dissolved in a 1:1 mixture of CH₂Cl₂ and CH₃OH (8 mL) and N-Chlorsuccinimid (240 mg, 1.8 mmol) added. Then it was stirred under N₂ for 3 h at ambient temperature. After the end of the reaction a saturated solution of NaCl (10 mL) was added and repeatedly extracted with CH₂Cl₂. The pooled organic phases were dried over Na₂SO₄, filtered and the solvent removed under vacuum. The crude product was purified using flash-chromatography (3 cm, cHex:EA 9:1, 10 mL, R_{f}=0.61). Colourless Liquid, Yield 192 mg (60%).

HPLC: 97.2%, t_{R}=23.2 min.

C₈H₁₀BrClO₂S (285.6).

MS (EI): m/z=257/255/253 [M⁺-OCH₃], 176/174 [M⁺-OCH₃, —Br].

IR (Film): v (cm⁻¹)=3096 (C—H), 2930 (C—H), 1069 (C—O).

¹H-NMR (CDCl₃):

δ (ppm)=3.03 (d, J=5.2 Hz, 2H, ThCH₂CH), 3.40 (s, 6H, CH(OCH₃)₂), 4.49 (t, J=5.2 Hz, 1H, ThCH₂CH), 6.76 (s, 1H, 4-H—Th).

### Example N

### 2-[3-Bromo-5-(α-hydroxybenzyl)thiophene-2-yl]acetaldehyde-dimethylacetal

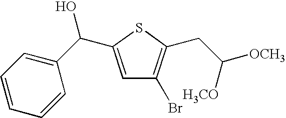

Example C (146 mg, 0.58 mmol) was dissolved under N₂ in THF (5 mL) and cooled to −40° C. A solution of LDA (2 M in THF, 520 μL, 1.04 mmol) was slowly added while stirring and the mixture was stirred for a further 10 min. Following that benzaldehyde (110 mg, 1.04 mmol), dissolved in THF (0.5 mL) was added. After 30 min the mixture was heated to ambient temperature, a saturated solution of NaCl (10 mL) added and extracted with CH₂Cl₂ (3×5 mL). The organic phase was dried over Na2SO4, filtered and the solvent removed under vacuum. The crude product was purified using flash-chromatography (2 cm, cHex:EA 8:2, 10 mL, R_{f}=0.32). Colourless Liquid, Yield 259 mg (93%).

HPLC: 99.3%, t_{R}=21.5 min.

C₁₅H₁₇BrO₃S (357.3).

MS (EI): m/z=358/356 [M⁺], 327/325 [M⁺-OCH₃], 77 [Ph⁺].

IR (Film): v (cm⁻¹)=3413 (O—H), 2926 (C—H), 1040 (C—O), 698 (C—H).

¹H-NMR (CDCl₃):

δ (ppm)=2.40 (d, J=4.0 Hz, 1H, OH), 3.03 (dd, J=15.0/5.5 Hz, 1H, ThCH₂CH), 3.07 (dd, J=15.0/5.5 Hz, 1H, ThCH₂CH), 3.37 (s, 6H, CH(OCH₃)₂), 4.53 (t, J=5.5 Hz, 1H, ThCH₂CH), 5.93 (d, J=4.0 Hz, 1H, CHOH), 6.67 (s, 1H, 4-H—Th), 7.28-7.43 (m, 5H, Ph-H).

### Example O

### 2-(5-Benzyl-3-bromothiophene-2-yl)acetaldehyde-dimethylacetal

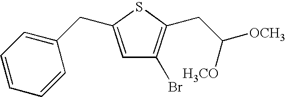

Example N (60 mg, 0.17 mmol) was dissolved in CH₂Cl₂ (2 mL) and n-butylsilane (17.8 mg, 0.2 mmol) followed by tris(pentafluorphenyl)borane (5.1 mg, 10 μmol) were added. After 8 h of stirring at ambient temperature 2 M NaOH (5 mL) were added and extracted with CH₂Cl₂ (3×5 mL). The organic phase was dried over Na₂SO₄, filtered and the solvent removed under vacuum. The crude product was purified using flash-chromatography (2 cm, cHex:EA 9:1, 5 mL, R_{f}=0.65). Colourless Oil, Yield 33.3 mg (58%).

HPLC: 96.3%, t_{R}=24.6 min.

C₁₅H₁₇BrO₂S (341.3).

MS (EI): m/z=342/340 [M⁺], 311/309 [M⁺-OCH₃], 267/265 [M⁺-CH(OCH₃)₂].

IR (Film): v (cm⁻¹)=3027 (C—H), 2930 (C—H), 1058 (C—O), 697 (C—H).

¹H-NMR (CDCl₃):

δ (ppm)=3.03 (d, J=5.6 Hz, 2H, ThCH₂CH), 3.37 (s, 6H, CH(OCH₃)₂), 4.04 (s, 2H, ThCH₂Ph), 4.52 (t, J=5.6 Hz, 1H, ThCH₂CH), 6.59 (s, 1H, 4-H—Th), 7.21-7.27 (m, 3H, Ph-H), 7.29-7.34 (m, 2H, Ph-H).

### Example P

### 4-(1-Benzyl-4-hydroxypiperidine-4-yl)-5-(2,2-dimethoxyethyl)thiophenecarb-aldehyde-dimethylacetal

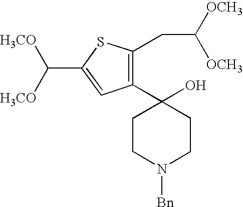

Example L (740 mg, 2.27 mmol) was dissolved in THF (20 mL) and under N₂ cooled to −84° C. A solution of n-butyllithium (1.5 M in hexane, 2.2 mL, 3.18 mmol) was added within 2-3 min while stirring, until-15 min later-1-Benzylpiperidine-4-one (430 mg, 2.27 mmol) was added. After 1 h of stirring at −90° C. the mixture was stirred a further 1 h at ambient temperature. The reaction was stopped by adding a saturated solution of NaCl (20 mL). Following that it was extracted with CH₂Cl₂ (3×5 mL). The pooled organic phases were dried over Na₂SO₄, filtered and the solvent removed under vacuum. The crude product was purified using flash-chromatography (4 cm, cHex:EA 1:1, 30 mL, R_{f}=0.14). Colourless oil, the yield was determined after cyclisation from EX P to Example 37.

MS (EI): m/z=435 [M⁺], 404 [M⁺-OCH₃], 344 [M⁺-CH₂Ph].

C₂₃H₃₃NO₅S (435.6).

IR (Film): v (cm⁻¹)=3455 (O—H), 2935 (C—H), 1050 (C—O), 698 (C—H).

¹H-NMR (CDCl₃):

δ (ppm)=1.68-1.74 (m, 2H, N(CH₂CH₂)₂), 2.08 (td, J=13.2/4.0 Hz, 2H, N(CH₂CH₂)₂), 2.46 (td, J=12.4/2.4 Hz, 2H, N(CH₂CH₂)₂), 2.68-2.74 (m, 2H, N(CH₂CH₂)₂), 3.35 (s, 12H, 2×CH(OCH₃)₂), 3.40 (d, J=5.6 Hz, 2H, ThCH₂CH), 3.55 (s, 2H, NCH₂Ph), 3.80 (s, 1H, OH), 4.49 (t, J=5.6 Hz, 1H, ThCH₂CH), 5.48 (s, 1H, ThCH), 6.88 (s, 1H, 3-H—Th), 7.20-7.37 (m, 5H, Ph-H).

### Example Q

### 2-[3-(1-Benzyl-4-hydroxypiperidine-4-yl)-5-chlorothiophene-2-yl]acetaldehyde-dimethylacetal

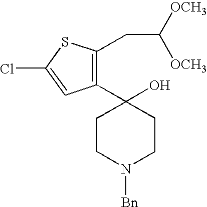

Example M (180 mg, 0.63 mmol) was dissolved in THF (15 mL) and cooled under N₂ to −78° C. A solution of n-butyllithium (1.5 M in hexane, 630 μL, 0.94 mmol) was added within 2-3 min while stirring, until-15 min later-1-Benzylpiperidine-4-one (120 mg, 0.63 mmol) was added. After 1 h of stirring at −78° C. the mixture was stirred a further 1 h at ambient temperature. The reaction was stopped by adding H₂O (20 mL). After extraction with CH₂Cl₂ (3×5 mL) the pooled organic phases were dried over Na₂SO₄, filtered and the solvent removed under vacuum. The crude product was purified using flash-chromatography (3 cm, cHex:EA 7:3, 10 mL, R₁=0.28). Colourless Oil, the yield was determined after cyclisation from EX Q to Example 38.

C₂₀H₂₆ClO₃S (395.9).

MS (EI): m/z=397/395 [M⁺], 366/364 [M⁺-OCH₃], 306/304 [M⁺-CH₂Ph].

IR (Film): v (cm⁻¹)=3444 (O—H), 3061 (C—H), 2921 (C—H), 1042 (C—O), 697 (C—H).

¹H-NMR (CDCl₃):

δ (ppm)=1.70 (dd, J=14.4/2.0 Hz, 2H, N(CH₂CH₂)₂), 2.02 (td, J=12.8/4.4 Hz, 2H, N(CH₂CH₂)₂), 2.43 (td, J=11.6/2.4 Hz, 2H, N(CH₂CH₂)₂), 2.67-2.74 (m, 2H, N(CH₂CH₂)₂), 3.35 (d, J=5.2 Hz, 2H, ThCH₂CH), 3.37 (s, 6H, CH(OCH₃)₂), 3.55 (s, 2H, NCH₂Ph), 3.69 (s, 1H, OH), 4.46 (t, J=5.2 Hz, 1H, ThCH₂CH), 6.72 (s, 1H, 4-H—Th), 7.19-7.36 (m, 5H, Ph-H).

### Example R

### 2-[5-Benzyl-3-(1-benzyl-4-hydroxypiperidine-4-yl)-5-benzylthiophene-2-yl]acetaldehyde-dimethylacetal

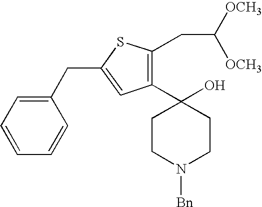

Example O (250 mg, 0.74 mmol) was dissolved in THF (15 mL) and cooled under N₂ to −78° C. A solution of n-butyllithium (1.5 M in hexane, 0.7 mL, 1.04 mmol) was added within 2-3 min while stirring, until-15 min later-1-Benzylpiperidine-4-one (140 mg, 0.74 mmol) was added. After 2 h of stirring at −78° C. the mixture was stirred a further 1 h at ambient temperature. The reaction was stopped by adding a saturated solution of NaCl (20 mL). After extraction with CH₂Cl₂ (3×5 mL) the pooled organic phases were dried over Na₂SO₄, filtered and the solvent removed under vacuum. The crude product (approx. 3.31 g) was purified using flash-chromatography (3 cm, cHex:EA 7:3, 30 mL, R_{f}=0.2). Colourless oil, the yield was determined after cyclisation from EX R to Example 39.

MS (EI): m/z=451 [M⁺], 420 [M⁺-OCH₃], 360 [M⁺-CH₂Ph].

C₂₇H₃₃NO₃S (451.6).

IR (Film): v (cm⁻¹)=3451 (O—H), 3026 (C—H), 2923 (C—H), 1039 (C—O), 697 (C—H).

¹H-NMR (CDCl₃): δ (ppm)=1.48-1.55 (m, 2H, N(CH₂CH₂)₂), 2.03 (td, J=13.2/4.4 Hz, 2H, N(CH₂CH₂)₂), 2.41-2.50 (m, 2H, N(CH₂CH₂)₂), 2.68-2.76 (m, 2H, N(CH₂CH₂)₂), 3.35 (d, J=5.2 Hz, 2H, ThCH₂CH), 3.36 (s, 6H, CH(OCH₃)₂), 3.55 (s, 2H, NCH₂Ph), 3.82 (s, 1H, OH), 4.01 (s, 2H, ThCH₂Ph), 4.47 (t, J=5.2 Hz, 1H, ThCH₂CH), 6.58 (s, 1H, 4-H—Th), 7.18-7.38 (m, 10H, Ph-H).

### Example 37

### (1-Benzyl-6′-methoxy-6′,7′-dihydrospiro[piperidine-4,4′-thieno[3.2-c]pyrano]-2-carbaldehyde-dimethylacetal

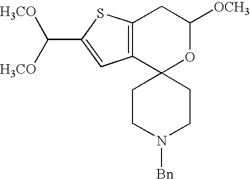

Example P (ca. 460 mg, non-purified) was dissolved in MeOH (15 mL) and trimethyl-orthoformiate (2 mL) added. After adding p-toluene-sulfonic acid (260 mg, 1.37 mmol) the mixture was stirred under N₂ for 45 min at ambient temperature. For purification the mixture was made alkaline with 2 M NaOH and extracted with CH₂Cl₂ (3×5 mL). The organic phases were pooled and dried over Na₂SO₄, filtered and the solvent removed under vacuum. The crude product was purified using flash-chromatography (3 cm, cHex:EA 7:3, 15 mL, R_{f}=0.23 (cHex:EA 1:1)). Colourless solid, MP 71° C., yield 287 mg (31.5% with 2 steps).

HPLC: 96.4%, t_{R}=16.9 min

C₂₂H₂₉NO₄S (403.5).

MS (EI): m/z=403 [M⁺], 372 [M⁺-OCH₃], 328 [M⁺-CH(OCH₃)₂], 312 [M⁺-CH₂Ph].

IR (Film): v (cm⁻¹)=2924 (C—H), 2828 (C—H), 1049 (C—O), 698 (C—H).

¹H-NMR (CDCl₃):

δ (ppm)=1.81 (dd, J=13.6/2.8 Hz, 1H, N(CH₂CH₂)₂), 1.85 (td, J=12.0/4.0 Hz, 1H, N(CH₂CH₂)₂), 1.94 (dd, J=13.6/2.8 Hz, 1H, N(CH₂CH₂)₂), 2.08 (td, J=13.6/4.8 Hz, 1H, N(CH₂CH₂)₂), 2.42 (td, J=12.0/3.2 Hz, 1H, N(CH₂CH₂)₂), 2.51 (td, J=12.0/2.8 Hz, 1H, N(CH₂CH₂)₂), 2.72-2.79 (m, 2H, N(CH₂CH₂)₂), 2.74 (dd, J=15.6/7.2 Hz, 1H, ThCH₂CH), 2.94 (dd, J=15.6/3.6 Hz, 1H, ThCH₂CH), 3.35 (s, 6H, CH(OCH₃)₂), 3.55 (s, 3H, ThCH₂CHOCH₃), 3.57 (d, J=16.0 Hz, 1H, NCH₂Ph), 3.59 (d, J=16.0 Hz, 1H, NCH₂Ph), 4.86 (dd, J=7.2/3.6 Hz, 1H, ThCH₂CH), 5.51 (s, 1H, ThCH), 6.79 (s, 1H, 3′-H—Th), 7.19-7.32 (m, 5H, Ph-H).

### Example 38

### 1-Benzyl-2′-chloro-6′-methoxy-6′,7′-dihydrospiro[piperidine-4,4′-thieno[3.2-c]-pyrano]

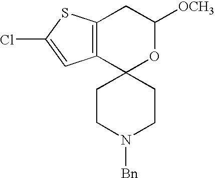

Example Q (ca. 120 mg, non-purified) was dissolved in MeOH (6 mL) and trimethylorthoformiate (0.5 mL) added. After adding p-toluene-sulfonic acid (120 mg, 0.63 mmol) the mixture was stirred under N₂ for 3 h at ambient temperature. For purification the mixture was made alkaline with 2 M NaOH and H₂O (10 mL) added. Following that it was extracted with CH₂Cl₂ (3×5 mL). The organic phases were pooled and dried over Na₂SO₄, filtered and the solvent removed under vacuum. The crude product was purified using flash-chromatography (2 cm, cHex:EA 7:3, 10 mL.

R_{f}=0.36). Colourless Solid, MP 117° C., Yield 77 mg (33% in 2 steps).

HPLC: 98.0%, t_{R}=19.8 min.

C₁₉H₂₂ClNO₂S (363.6).

MS (EI): m/z=365/363 [M⁺], 334/332 [M⁺-OCH₃], 272/274 [M⁺-CH₂Ph].

IR (Film): v (cm⁻¹)=3069 (C—H), 2917 (C—H), 1059 (C—O), 700 (C—H).

¹H-NMR (CDCl₃):

δ (ppm)=1.81 (dd, J=13.2/2.8 Hz, 1H, N(CH₂CH₂)₂), 1.83 (td, J=13.6/4.4 Hz, 1H, N(CH₂CH₂)₂), 1.93 (dd, J=14.0/2.8 Hz, 1H, N(CH₂CH₂)₂), 2.01 (td, J=13.6/4.4 Hz, 1H, N(CH₂CH₂)₂), 2.41 (td, J=11.6/2.8 Hz, 1H, N(CH₂CH₂)₂), 2.50 (td, J=11.6/2.8 Hz, 1H, N(CH₂CH₂)₂), 2.72-2.79 (m, 2H, N(CH₂CH₂)₂), 2.74 (dd, J=15.6/7.2 Hz, 1H, ThCH₂CH), 2.86 (dd, J=15.6/3.2 Hz, 1H, ThCH₂CH), 3.54 (s, 3H, OCH₃), 3.54 (d, J=12.8 Hz, 1H, NCH₂Ph), 3.58 (d, J=12.8 Hz, 1H, NCH₂Ph), 4.86 (dd, J=7.2/3.2 Hz, 1H, ThCH₂CH), 6.62 (s, 1H, 3′-H —Th), 7.20-7.35 (m, 5H, Ph-H).

### Example 39

### 1,2′-Dibenzyl-6′-methoxy-6′,7′-dihydrospiro[piperidine-4,4′-thieno[3.2-c]pyrano]

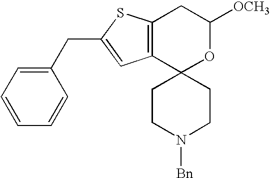

Example R (ca. 185 mg, non-purified) was dissolved in MeOH (6 mL) and trimethylorthoformiate (3 mL) added. After adding p-toluene-sulfonic acid (120 mg, 0.61 mmol) the mixture was stirred under N₂ for 2.5 h at ambient temperature. For purification the mixture was made alkaline with 2 M NaOH and H₂O (10 mL) was added. Following that it was extracted with CH₂Cl₂ (3×5 mL). The organic phases were dried over Na₂SO₄, filtered and the solvent removed under vacuum. The crude product was purified using flash-chromatography (2 cm, cHex:EA 7:3, 15 mL, R_{f}=0.22). Colourless, oily substance, yield 82 mg (26% in 2 steps).

HPLC: 98.3%, t_{R}=21.8 min.

C₂₆H₂₉NO₂S (419.6).

MS (EI): m/z=419 [M⁺], 388 [M⁺-OCH₃], 328 [M⁺-CH₂Ph].

IR (Film): v (cm⁻¹)=3027 (C—H), 2922 (C—H), 1060 (C—O), 696 (C—H).

¹H-NMR (CDCl₃):

δ (ppm)=1.79 (dd, J=13.6/2.8 Hz, 1H, N(CH₂CH₂)₂), 1.82 (td, J=14.0/4.4 Hz, 1H, N(CH₂CH₂)₂), 1.93 (dd, J=14.0/2.8 Hz, 1H, N(CH₂CH₂)₂), 2.03 (td, J=13.6/4.4 Hz, 1H, N(CH₂CH₂)₂), 2.40 (td, J=11.6/2.8 Hz, 1H, N(CH₂CH₂)₂), 2.49 (td, J=11.6/2.4 Hz, 1H, N(CH₂CH₂)₂), 2.70-2.78 (m, 2H, N(CH₂CH₂)₂), 2.75 (dd, J=15.6/7.2 Hz, 1H, ThCH₂CH), 2.86 (dd, J=15.6/3.2 Hz, 1H, ThCH₂CH), 3.54 (d, J=16.4 Hz, 1H, NCH₂Ph), 3.54 (s, 3H, OCH₃), 3.56 (d, J=16.4 Hz, 1H, NCH₂Ph), 4.04 (s, 2H, ThCH₂Ph), 4.83 (dd, J=7.2/3.2 Hz, 1H, ThCH₂CH), 6.45 (s, 1H, 3′-H—Th), 7.19-7.35 (m, 10H, Ph-H).

### Example 40

### (1-Benzyl-6′-methoxy-6′,7′-dihydrospiro[piperidine-4,4′-thieno[3.2-c]pyrano]-2-carbaldehyde

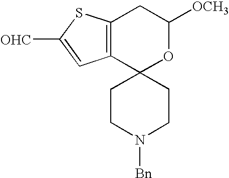

Example 37 (287 mg, 0.71 mmol) was dissolved in CH₃CN (15 mL) and HCl (1 M, 1 mL) added. After 15 min the mixture was made alkaline with 2 M NaOH and the aqueous phase repeatedly extracted with CH₂Cl₂. Following that the pooled organic phases were dried over Na2SO4, filtered and the solvent removed under vacuum. The purification of the crude product was done using flash-chromatography (2 cm, cHex:EA 1:1, 15 mL, R_{f}=0.14). Colourless viscous oil, 244 mg (96%).

HPLC: 98.0%, t_{R}=17.1 min.

C₂₀H₂₃NO₃S (357.5).

MS (EI): m/z=357 [M⁺], 326 [M⁺-OCH₃], 265 [M⁺-CH₂Ph].

IR (Film): v (cm⁻¹)=3027 (C—H), 2910 (C—H), 1665 (C═O), 1059 (C—O), 698 (C—H).

¹H-NMR (CDCl₃):

δ (ppm)=1.84-2.14 (m, 3H, N(CH₂CH₂)₂), 2.01 (td, J=13.6/4.8 Hz, 1H, N(CH₂CH₂)₂), 2.43 (td, J=11.6/3.6 Hz, 1H, N(CH₂CH₂)₂), 2.53 (td, J=12.0/2.8 Hz, 1H, N(CH₂CH₂)₂), 2.65-2.73 (m, 2H, N(CH₂CH₂)₂), 2.91 (dd, J=16.4/6.4 Hz, 1H, ThCH₂CH, 3.06 (dd, J=16.4/3.2 Hz, 1H, ThCH₂CH), 3.55 (s, 3H, OCH₃), 3.58 (s, 2H, NCH₂Ph), 4.92 (dd, J=6.4/3.2 Hz, 1H, ThCH₂CH), 7.18-7.37 (m, 5H, Ph-H), 7.49 (s, 1H, 3′-H—Th), 9.80 (s, 1H, ThCHO).

### Example 41

### (1-Benzyl-6′-methoxy-6′,7′-dihydrospiro[piperidine-4,4′-thieno[3.2-c]pyrano]-2′-yl)-methanol

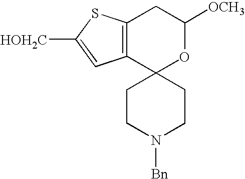

The aldehyde 40 (60 mg, 0.16 mmol) was dissolved under N₂ in THF (3 mL) and cooled to −40° C. A solution of LiAlH₄ (1 M in THF, 84 μL, 0.084 mmol) was slowly added and the mixture stirred for 30 min. After adding a saturated solution of NaCl (10 mL) it was extracted repeatedly with CH₂Cl₂. The pooled organic phases were dried over Na2SO4, filtered and the solvent removed under vacuum. The crude product was purified using flash-chromatography (1 cm, cHex:EA 1:9, 10 mL, R_{f}=0.18). Colourless viscous oil, yield 30 mg (57%).

HPLC: 98.0%, t_{R}=16.0 min.

C₂₀H₂₅NO₃S (359.5).

MS (EI): m/z=359 [M⁺], 328 [M⁺-OCH₃] 268 [M⁺-CH₂Ph].

IR (Film): v (cm⁻¹)=3411 (O—H), 3024 (C—H), 2923 (C—H), 1027 (C—O), 698 (C—H).

¹H-NMR (CDCl₃):

δ (ppm)=1.80 (dd, J=13.6/2.4 Hz, 1H, N(CH₂CH₂)₂), 1.84-1.95 (m, 2H, N(CH₂CH₂)₂), 1.99 (s, 1H, OH), 2.07 (td, J=13.4/4.4 Hz, 1H, N(CH₂CH₂)₂), 2.43 (td, J=11.6/3.6 Hz, 1H, N(CH₂CH₂)₂), 2.52 (td, J=11.6/2.4 Hz, 1H, N(CH₂CH₂)₂), 2.74-2.79 (m, 2H, N(CH₂CH₂)₂), 2.81 (dd, J=16.0/7.2 Hz, 1H, ThCH₂CH), 2.94 (dd, J=16.0/3.6 Hz, 1H, ThCH₂CH), 3.54 (s, 3H, OCH₃), 3.55 (d, J=13.2 Hz, 1H, NCH₂Ph), 3.59 (d, J=13.2 Hz, 1H, NCH₂Ph), 4.72 (s, 2H, ThCH₂OH), 4.86 (dd, J=7.2/3.6 Hz, 1H, ThCH₂CH), 6.71 (s, 1H, 3′-H—Th), 7.18-7.36 (m, 5H, Ph-H).

### Example 42

### (1-Benzyl-6′-methoxy-6′,7′-dihydrospiro[piperidine-4,4′-thieno[3.2-c]pyrano]-2′-yl)-N,N-dimethylmethanamine

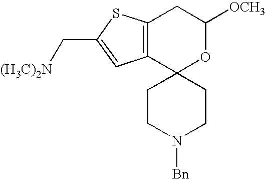

Example 40 (51 mg, 0.14 mmol) was dissolved in CH₂Cl₂ (4 mL), a solution of dimethylamine (2 M in THF, 280 μL, 0.56 mmol) and NaBH(OAc)₃ (55 mg, 0.27 mmol) added and stirred for 1.5 h at ambient temperature. After the reaction finished a saturated solution of NaCl (10 mL) was added and extracted with CH₂Cl₂ (3×5 mL). The organic phases were dried over Na2SO4, filtered and the solvent removed under vacuum. The crude product was purified using flash-chromatography (1 cm, cHex:EA:MeOH 2:6.5:1.5, 5 mL, R_{f}=0.10). Colourless oil, yield 41 mg (77%).

HPLC: 98.3%, t_{R}=13.9 min.

C₂₂H₃₀N₂O₂S (386.6).

MS (EI): m/z=386 [M⁺], 355 [M⁺-OCH₃], 342 [M⁺-N(CH₃)₂].

IR (Film): v (cm⁻¹)=2921 (C—H), 2813 (C—H), 1061 (C—O), 697 (C—H).

¹H-NMR (CDCl₃):

δ (ppm)=1.80 (dd, J=13.6/2.8 Hz, 1H, N(CH₂CH₂)₂), 1.89 (td, J=12.4/4.4 Hz, 1H, N(CH₂CH₂)₂), 1.94 (dd, J=14.0/2.8 Hz, 1H, N(CH₂CH₂)₂), 2.08 (td, J=14.0/4.4 Hz, 1H, N(CH₂CH₂)₂), 2.26 (s, 6H, N(CH₃)₂), 2.44 (td, J=11.6/3.2 Hz, 1H, N(CH₂CH₂)₂), 2.54 (td, J=11.6/2.4 Hz, 1H, N(CH₂CH₂)₂), 3.74-3.81 (m, 2H, N(CH₂CH₂)₂), 3.79 (dd, J=15.6/7.6 Hz, 1H, ThCH₂CH), 2.92 (dd, J=15.6/3.6 Hz, 1H, ThCH₂CH), 3.52 (d, J=13.6 Hz, 1H, ThCH₂N(CH₃)₂), 3.54 (s, 3H, OCH₃), 3.55 (d, J=13.6 Hz, 1H, ThCH₂N(CH₃)₂), 3.57 (d, J=13.2 Hz, 1H, NCH₂Ph), 3.60 (d, J=13.2 Hz, 1H, NCH₂Ph), 4.86 (dd, J=7.6/3.6 Hz, 1H, ThCH₂CH), 6.59 (s, 1H, 3′-H—Th), 7.20-7.37 (m, 5H, Ph-H).

### Example 43

### (E/Z)-1-Benzyl-6′-methoxy-6′,7′-dihydrospiro[piperidine-4,4′-thieno[3,2-c]pyrano]-2-carbaldehydroxim

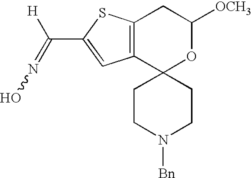

Example 40 (43 mg, 0.12 mmol) was dissolved in pyridine (2 mL) and Hydroxylamine-hydrochloride (20 mg, 0.3 mmol) added. After stirring for 30 min at 60° C. the pyridine was removed under vacuum and the oxime precipitated as colourless solid. Superfluous hydroxylamine hydrochloride and impurities were removed by repeated washing with CH₃OH. Colourless solid, MP 233° C., yield 31 mg (70%).

HPLC: 99.7%, t_{R}=17.0 min.

C₂₀H₂₄N₂O₂S (372.5).

MS (EI): m/z=372 [M⁺], 355 [M⁺-OH], 281 [M⁺-CH₂Ph].

IR (Film): v (cm⁻¹)=3028 (C—H), 2961 (C—H), 1622 (C═N), 1053 (C—O).

¹H-NMR (DMSO₆):

δ (ppm)=1.60-1.68 (m, 1H, N(CH₂CH₂)₂), 1.67-1.80 (m, 1H, N(CH₂CH₂)₂), 1.88-2.07 (m, 2H, N(CH₂CH₂)₂), 2.34-2.45 (m, 2H, N(CH₂CH₂)₂), 2.66 (td, J=16.4/6.8 Hz, 1H, N(CH₂CH₂)₂), 2.62-2.73 (m, 2H, ThCH₂CH), 2.95 (ddd, J=19.2/16.4/3.2 Hz, 1H, N(CH₂CH₂)₂), 3.42 (s, 3H, OCH₃), 3.52 (s (breit), 2H, NCH₂Ph), 4.89-4.95 (m, 1H, ThCH₂CH), 7.14 (s, 0.46H, (E) 3′-H—Th), 7.30 (s, 0.54H, (Z) 3′-H—Th), 7.32-7.40 (m, 5H, Ph-H), 7.70 (s, 0.54H, (Z) CH═NOH), 8.20 (s, 0.46H, (E) CH═NOH), 11.12 (s, 0.46H, (E) CH═NOH), 11.76 (s, 0.54H, (Z) CH═NOH). The exact identification of the 3′-thiophene-protons and the protons of CH— and OH of the oxim was done with gHSQC- and gHMBC-spectra and analogously to similar oxims. The ratio (E):(Z) of 46:54 was calculated by integrating the signals of the (E)/(Z)-OH-protons.

### Example 44

### 1-Benzyl-6′-methoxy-6′,7′-dihydrospiro[piperidine-4,4′-thieno[3,2-c]pyrano]-2-carbonitrile

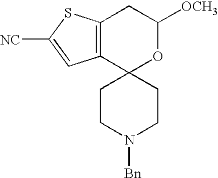

Example 40 (150 mg, 0.42 mmol) was dissolved in pyridine (4 mL) and hydroxylamine hydrochloride (50 mg, 0.72 mmol) added. After heating for 30 min at 60° C. phthalic acid anhydride (240 mg, 1.6 mmol) was added and the mixture was heated for a further 60 min at 90° C. For purification a saturated solution of NaCl was added and extracted with CH₂Cl₂ (3×5 mL). The pooled organic phases were dried over Na2SO4, filtered and the solvent removed under vacuum. The crude product was purified using flash-chromatography (3 cm, cHex:EA 4:6, 10 mL. R_{f}=0.37). Colourless solid, MP 154° C., yield 68 mg (67%).

Analysis of elements: calc.: C, 67.77; H, 6.26; N, 7.73; found.: C, 67.38; H, 6.34; N, 7.73.

HPLC: 99.7%, t_{R}=18.0 min.

C₂₀H₂₂N₂O₂S (354.4).

MS (EI): m/z=354 [M⁺], 323 [M⁺-OCH₃], 232 [M⁺-OCH₃, —CH₂Ph].

IR (Film): v (cm⁻¹)=3061 (C—H), 2936 (C—H), 2211 (C≡N), 1058 (C—O), 697 (C—H).

¹H-NMR (CDCl₃):

δ (ppm)=1.80-1.88 (m, 2H, N(CH₂CH₂)₂), 1.92 (dd, J=14.0/2.8 Hz, 1H, N(CH₂CH₂)₂), 2.03 (td, J=12.8/4.8 Hz, 1H, N(CH₂CH₂)₂), 2.42 (td, J=11.6/2.8 Hz, 1H, N(CH₂CH₂)₂), 2.51 (td, J=11.8/2.8 Hz, 1H, N(CH₂CH₂)₂), 2.74-2.83 (m, 2H, N(CH₂CH₂)₂), 2.87 (dd, J=16.4/6.4 Hz, 1H, ThCH₂CH), 3.10 (dd, J=16.4/3.6 Hz, 1H, ThCH₂CH), 3.56 (s, 3H, OCH₃), 3.57 (d, J=13.2 Hz, 1H, NCH₂Ph), 3.59 (d, J=13.2 Hz, 1H, NCH₂Ph), 4.92 (dd, J=6.4/3.6 Hz, 1H, ThCH₂CH), 7.33 (s, 1H, 3′-H—Th), 7.20-7.38 (m, 5H, Ph-H).

¹³C-NMR (CDCl₃):

δ (ppm)=31.8 (1C, ThCH₂CH), 36.3 (1C, N(CH₂CH₂)₂), 38.7 (1C, N(CH₂CH₂)₂), 49.1 (1C, N(CH₂CH₂)₂), 49.2 (1C, N(CH₂CH₂)₂), 56.8 (1C, OCH₃), 63.5 (1C, NCH₂Ph), 74.3 (1C, ThCO), 96.4 (1C, ThCH₂CH), 107.7 (1C, C-2′-Th), 114.6 (1C, CN), 127.3 (1C, para-C-Ph), 128.5 (2C, meta-C-Ph), 129.4 (2C, ortho-C-Ph-CH), 134.4 (1C, C-3′-Th), 138.5 (1C, Ph-C), 139.0 (1C, C-7a′-Th), 141.7 (1C, C-3a′-Th). The exact identification of the signals in the ¹³C-NMR-spectra was done with the help of gHSQC- and gHMBC-spectra.

### Example 45

### Methyl-1-benzyl-6′-methoxy-6′,7′-dihydrospiro[piperidine-4,4′-thieno[3,2-c]pyrano]-2-carboxylate

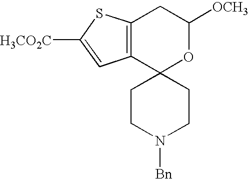

Example 44 (26 mg, 0.073 mmol) was dissolved in CH₃OH, H₂SO₄ conc. (0.5 mL) and 3 drops of H₂O added and heated for 22 h under reflux. After alkalinisation with NaOH (2 M, 7 mL) under cooling with ice the mixture was extracted with CH₂Cl₂ (3×5 mL). The organic phases were pooled dried over Na2SO4, filtered and the solvent removed under vacuum. The crude product was purified using flash-chromatography (1 cm, cHex:EA 7:3, 5 mL, R_{f}=0.17). Colourless solid, MP 136-138° C., yield 17.1 mg (60%).

HPLC: 98.6%, t_{R}=18.1 min.

C₂₁H₂₅NO₄S (387.5).

MS (EI): m/z=387 [M⁺], 356 [M⁺-OCH₃], 296 [M⁺-CH₂Ph].

IR (Film): v (cm⁻¹)=3023 (C—H), 2944 (C—H), 1701 (C═O), 1070 (C—O), 697 (C—H).

¹H-NMR (CDCl₃):

δ (ppm)=1.82 (dd, J=13.2/2.8 Hz, 1H, N(CH₂CH₂)₂), 1.86-1.95 (m, 2H, N(CH₂CH₂)₂), 2.08 (td, J=13.2/4.4 Hz, 1H, N(CH₂CH₂)₂), 2.42 (td, J=11.6/3.6 Hz, 1H, N(CH₂CH₂)₂), 2.51 (td, J=13.2/2.8 Hz, 1H, N(CH₂CH₂)₂), 2.73-2.79 (m, 2H, N(CH₂CH₂)₂), 2.86 (dd, J=16.4/7.2 Hz, 1H, ThCH₂CH), 3.00 (dd, J=16.0/3.2 Hz, 1H, ThCH₂CH), 3.55 (s, 3H, OCH₃), 3.55 (d, J=13.2 Hz, 1H, NCH₂Ph), 3.58 (d, J=13.2 Hz, 1H, NCH₂Ph), 3.85 (s, 3H, CO₂CH₃), 4.90 (dd, J=7.2/3.2 Hz, 1H, ThCH₂CH), 7.21-7.36 (m, 5H, Ph-H), 7.53 (s, 1H, 3′-H—Th).

### Example 46

### 1-(1-Benzyl-6′-methoxy-6′,7′-dihydrospiro[piperidine-4,4′-thieno[3.2-c]pyrano]-2-yl)-1-phenylmethanol

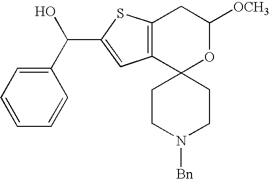

Example 40 (125 mg, 0.35 mmol) was dissolved under N₂ in THF (15 mL) and cooled on ice to 0° C. A solution of phenylmagnesiumbromide (1 M in THF, 700 μL, 0.7 mmol) was slowly added while stirring and the mixture stirred for a further 15 min. Following that a saturated solution of NaCl (10 mL) was added and extracted with CH₂Cl₂ (3×5 mL). The organic phase was dried over Na₂SO₄, filtered and the solvent removed under vacuum. The crude product was purified using flash-chromatography (2 cm, cHex:EA 1:1, 20 mL, R_{f}=0.15). Colourless solid, MP 78-80° C., yield 150 mg (98%).

HPLC: 99.4%, t_{R}=19.3 min.

C₂₆H₂₉NO₃S (435.6).

MS (EI): m/z=435 [M⁺], 313 [M⁺-OCH₃, —CH₂Ph], 91 [⁺CH₂Ph].

IR (Film): v (cm⁻¹)=3428 (O—H), 3083 (C—H), 1060 (C—O), 697 (C—H).

¹H-NMR (CDCl₃):

δ (ppm)=1.75-1.85 (m, 2H, N(CH₂CH₂)₂), 1.86-1.93 (m, 1H, N(CH₂CH₂)₂), 2.00 (td, J=13.2/4.4 Hz, 1H, N(CH₂CH₂)₂), 2.38 (td, J=11.8/2.8 Hz, 1H, N(CH₂CH₂)₂), 2.39 (td, J=11.8/2.8 Hz, 1H, N(CH₂CH₂)₂), 2.43-2.52 (m, 2H, N(CH₂CH₂)₂), 2.76 (dd, J=15.6/7.2 Hz, 0.5H, ThCH₂CH), 2.78 (dd, J=15.6/7.2 Hz, 0.5H, ThCH₂CH), 2.89 (dd, J=15.6/3.2 Hz, 0.5H, ThCH₂CH), 2.90 (dd, J=15.6/3.2 Hz, 0.5H, ThCH₂CH), 3.53 (s, 3H, OCH₃) 3.54 (s, 2H, NCH₂Ph), 4.82-4.86 (m, 1H, ThCH₂CH), 5.93 (s, 1H, ThCHPh), 6.54 (s, 0.5H, 3′-H—Th), 6.57 (s, 0.5H, 3′-H—Th), 7.22-7.45 (m, 10H, Ph-H). A signal for the proton of the OH-group can not be seen. The diastereomers are present in a ratio of 54:46.

### Example 47

### 1-(1-Benzyl-6′-methoxy-6′,7′-dihydrospiro[piperidine-4,4′-thieno[3.2-c]pyrano]-2-yl)-1-phenylmethanone

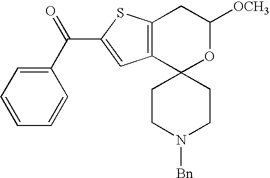

Example 46 (50 mg, 0.11 mmol) was dissolved in CH₂Cl₂ (4 mL) and “Molsieb 4 Å” (molecular sieve) (100 mg) added. After adding 1-methylmorpholine-N-oxide (24 mg, 0.2 mmol) and tetra-propylammoniumperruthenate (2.4 mg, 0.07 μmol) it was stirred for 20 min at ambient temperature. For purification a saturated solution of NaCl was added (10 mL) and extracted with CH₂Cl₂ (3×5 mL). The organic phase was dried over Na₂SO₄ and after filtration the solvent was removed under vacuum. The crude product was purified using flash-chromatography (2 cm, cHex:EA 7:3, 10 mL, R_{f}=0.2). Colourless solid, MP 91° C., yield 36 mg (72%).

HPLC: 98.0%, ambient temperature=20.2 min.

C₁₆H₂₇BrO₃S (433.6).

MS (EI): m/z=433 [M⁺], 402 [M⁺-OCH₃], 342 [M⁺-CH₂Ph].

IR (Film): v (cm⁻¹)=2921 (C—H), 1631 (C═O), 1054 (C—O), 697 (C—H).

¹H-NMR (CDCl₃):

δ (ppm)=1.81-1.91 (m, 2H, N(CH₂CH₂)₂), 1.97 (dd, J=14.4/2.8 Hz, 1H, N(CH₂CH₂)₂), 2.04 (td, J=13.6/4.4 Hz, 1H, N(CH₂CH₂)₂), 2.43 (td, J=12.4/2.8 Hz, 1H, N(CH₂CH₂)₂), 2.52 (td, J=12.4/2.8 Hz, 1H, N(CH₂CH₂)₂), 2.75-2.82 (m, 2H, N(CH₂CH₂)₂), 2.92 (dd, J=16.4/6.8 Hz, 1H, ThCH₂CH), 3.07 (dd, J=16.4/3.2 Hz, 1H, ThCH₂CH), 3.54 (d, J=14.0 Hz, 1H, NCH₂Ph), 3.56 (s, 3H, OCH₃), 3.58 (d, J=14.0 Hz, 1H, NCH₂Ph), 4.93 (dd, J=6.8/3.2 Hz, 1H, ThCH₂CH), 7.19-7.33 (m, 5H, CH₂Ph-H), 7.33 (s, 1H, 3′-H—Th), 7.49 (t, J=7.8 Hz, 2H, meta-H-PhCO), 7.59 (t, J=7.8 Hz, 1H, para-H-PhCO), 7.79 (d, J=7.8 Hz, 2H, ortho-H-PhCO).

### Biological Activity

### A) In-Vitro

Some representative compounds of the invention were tested for their activity as sigma (sigma-1 and sigma-2) inhibitors. The following protocols were followed:

### Sigma-1 (Version A)

Brain membrane preparation and binding assays for the σ1-receptor were performed as described (DeHaven-Hudkins et al., 1992) with some modifications. In brief, guinea pig brains were homogenized in 10 vols. (w/v) of Tris-HCl 50 mM 0.32 M sucrose, pH 7.4, with a Kinematica Polytron PT 3000 at 15000 r.p.m. for 30 s. The homogenate was centrifuged at 1000 g for 10 min at 4° C. and the supernatants collected and centrifuged again at 48000 g for 15 min at 4° C. The pellet was resuspended in 10 volumes of Tris-HCl buffer (50 mM, pH 7.4), incubated at 37° C. for 30 min, and centrifuged at 48000 g for 20 min at 4° C. Following this, the pellet was resuspended in fresh Tris-HCl buffer (50 mM, pH 7.4) and stored on ice until use.

The radioligand used was [³H]-(+)-pentazocine at 3.0 nM and the final volume was 200 μl. The incubation was initiated with the addition of 100 μl of membrane at a final tissue concentration of approximately 5 mg tissue net weight/mL and the incubation time was 150 min. at 37° C. After incubation, the membranes were collected onto pretreated glass fiber filterplate (MultiScreen-FC, Millipore), with polyethylenimine 0.1%. The filters were washed two times with 200 μl of washing buffer (50 mM Tris Cl, pH=7.4) and then 25 μl of Ecoscint H liquid scintillation cocktail were added.

Microplates were allowed to set for several hours and then quantified by liquid scintillation spectrophotometry (1450 Microbeta, Wallac). Nonspecific binding was determined with 1 μM haloperidol.

The Microbeta reader gave the counts per minute (cpm) per each well that were processed in Excel work sheet to obtain means of duplicates. The Specific Binding value was obtained substracting Non-Specific Binding (NSB) from Total Binding (TB).

Percentages of Specific Bound for each different compound concentration were calculated from duplicates cpm means as follow:

$$\frac{Compound - NSB}{Specific Bound} \times 100 \qquad [ Equation 1 ]$$

The values were used for non-linear IC₅₀ (nM) calculation and graph representation. Inhibition constant (K₁) was calculated from IC₅₀ according the Cheng-Prussof Equation:

$${K}_{i} = \frac{{IC}_{50}}{1 + \frac{[ L ]}{{K}_{d}}} \qquad [ Equation 2 ]$$

In which [L] means the radioligand concentration, determinated from the experimental total counts (dpm) using the Specific Activity of the radioligand, and K_{d} the disotiation constant of the radioligand.

The historical value of the saturation constant K_{d} of [³H]-(+)pentazocine was 3.1 nM.

### Sigma 1 (Version B)

In brief the σ₁-receptor preparation was prepared from guinea pig brain. The brains were homogenized in 5 to 6 times of volume sucrose solution (0.32M) and homogenized. The homogenate was centrifuged at 2900 rpm, 4° C., 10 min). the supernatant was centrifuged again (23500×g, 4° C., 20 min). The pellet was resuspended in Tris-buffer, incubated for 30 min at room temperature and centrifuged f (23500×g, 4° C., 20 min). The pellet was resuspended in cold TRIS-buffer and homogenized. Then the protein content was measured (approx. 1.5 mg/mL) and the homogenate frozen at −80° C. for later use.

The radioligand used was [³H]-(+)-Pentazocin in TRIS-buffer. In a volume of 200 μL 50 μL TRIS-buffer, 50 μL compound solution of varying concentration, 50 μL radioligand-solution (8 nM; resulting in 2 nM in the assay) and finally 50 μL of receptor preparation (approx. 1.5 mg/mL) were given into a well of a microplate equipped with a filter. The plate was closed and stirred for 2.5 h at 37° C. and 500 rpm. Following that the solvents were removed by a harvester through the filter. After rinsing with H₂O the filter was measured in a scintillation counter ([³H]-protocol).

### Sigma-2 (Version A)

Brain membrane preparation and binding assays for the σ2-receptor were performed as described (Ronsisvalle et al., 2001) with some modifications. Brain from 6 male Guinea pigs (400 g aprox) were carefully dissected on ice and weighed after discarding cerebellum. Homogenates were prepared in 10 volumes of cold 10 mM Tris-HCl, 0.35 M sucrose, pH 7.4 buffer with Polytron PT3000 and the suspension was centrifuged at 1,000×g for 10 minutes. Supernatants were reserved on ice and each pellet was resuspended in 4 ml of 10 mM Tris-HCl, 0.35 M sucrose, pH 7.4 buffer, homogenized and centrifuged again at 1,000×g for 10 minutes. All supernatants were mixed and the obtained pool was centrifuged at 33,000×g for 15 min. Obtained pellets were resuspended in 1.3 ml of 10 mM Tris-HCl, pH 7.4 buffer and incubated at 25° C. during 15 min and centrifuged again at 33,000×g for 15 min. Obtained pellets were resuspended in 1 ml of 10 mM Tris-HCl, pH 7.4 buffer, pooled and homogenized using Polytron PT3000 at 13500 rpm during 30 sec.

For each experiment, sigma 2 enriched membrane pellets were brought to room temperature and gently resuspended in binding buffer containing 50 mM Tris-HCl, pH 8.0 to obtain 200 μg protein/100 μl.

The radioligand used was [³H]-DTG at 10 nM and the final incubation volume was 200 μl. The incubation was initiated with the addition of 100 μl of membrane (200 μg) and the incubation time was 2 hours. at 25° C. NSB (non specific binding) was achieved with 5 μM Di-o-tolylguanidine (DTG) added in 40 μl volume. Sigma 1 receptors were blocked with the sigma 1 ligand (+)-SKF10047 at 400 nM. After incubation, the membranes were collected onto pretreated glass fiber filterplate (MultiScreen-FC, Millipore), with polyethylenimine 0.5%. The filters were washed two times with 200 μl of washing buffer (10 mM Tris-HCl, pH 8.0) and then 25 μl of Ecoscint H liquid scintillation cocktail were added. Microplates were allowed to set for several hours and then quantified by liquid scintillation spectrophotometry (1450 Microbeta, Wallac).

The Microbeta reader gave the counts per minute (cpm) per each well that were processed in Excel work sheet to obtain means of duplicates. The Specific Binding value was obtained substracting Non-Specific Binding (NSB) from Total Binding (TB).

Percentages of Specific Bound for each different compound concentration were calculated from duplicates cpm means as follow:

$$\frac{Compound - NSB}{Specific Bound} \times 100 \qquad [ Equation 1 ]$$

The values were used for non-linear IC₅₀ (nM) calculation and graph representation. Inhibition constant (Kᵢ) was calculated from IC₅₀ according the Cheng-Prussof Equation:

$${K}_{i} = \frac{{IC}_{50}}{1 + \frac{[ L ]}{{K}_{d}}} \qquad [ Equation 2 ]$$

In which [L] means the radioligand concentration, determinated from the experimental total counts (dpm) using the Specific Activity of the radioligand, and K_{d} the disotiation constant of the radioligand.

The historical value of the saturation constant K_{d} of [³H]-DTG was 23.3 nM.

### REFERENCES

DeHaven-Hudkins, D. L., L. C. Fleissner, and F. Y. Ford-Rice, 1992, “Characterization of the binding of [³H](+)pentazocine to σ recognition sites in guinea pig brain”, Eur. J. Pharmacol. 227, 371-378.

Ronsisvalle G, Prezzavento O, Marrazzo A, Vittorio F, Bousquet E, Di Toro R, Spampinato S. 2001 Synthesis and binding affinity of cis-(−)- and cis-(+)-N-ethyleneamino-N-nordeoxymetazocine and cis-(−)-N-normetazocine analogues at sigma1, sigma2 and kappa opioid receptors. Eur J Pharm Sci. 12(3), 277-84.

### Sigma 2 (Version B)

In brief the σ₂-Receptorpreparation was prepared from rat liver. The livers were homogenized in 5 to 6 times of volume sucrose solution (0.32M) and homogenized. The homogenate was centrifuged at 2900 rpm, 4° C., 10 min). the supernatant was centrifuged again (31000×g, 4° C., 20 min). The pellet was resuspended in TRIS-buffer, incubated for 30 min at room temperature while stirring and centrifuged f (31000×g, 4° C., 20 min). The pellet was resuspended in cold TRIS-buffer pH 8 and homogenized. Then the protein content was measured (approx. 2 mg/mL) and the homogenate frozen at −80° C. for later use.

The radioligand used was [³H]-Ditolylguanidin in TRIS-buffer pH 8. The σ₁-receptor binding sites were masked through (+)-Pentazocin-solution in TRIS-Puffer pH 8.

In a volume of 200 μL 50 μL compound solution of varying concentration, 50 μL (+)-Pentazocin-solution (2 NM; resulting in 500 nM in the assay), 50 μL radioligand-solution (12 nM; resulting in 3 nM in the assay) and finally 50 μL of receptor preparation (approx. 2 mg/mL) were given into a well of a microplate equipped with a filter. The plate was closed and stirred for 2 h at room temperature and 500 rpm. Following that the solvents were removed by a harvester through the filter. After rinsing with H₂O the filter was measured in a scintillation counter ([³H]-protocol).

Some of the results obtained (through the version B) are shown in table (I).

TABLE (I)

| | | | Binding σ2 |
|---|---|---|---|
| | | Binding σ1 | Ki [nM] or |
| Example | Formula | Ki [nM] | *[Inhibition at 1 μM] |
| 2 | 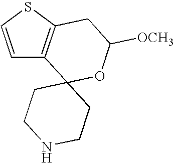 | 602 | 17%* |
| 3 | 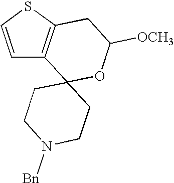 | 0.32 ± 0.16 | 1260 |
| 4 | 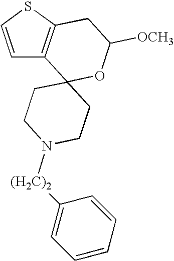 | 5.89 ± 5.10 | 191 |
| 5 | 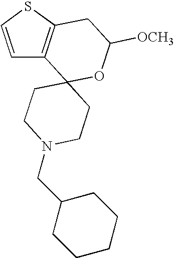 | 0.29 ± 0.079 | 25.4 ± 13.5 |
| 6 | 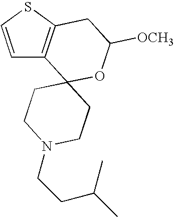 | 0.96 ± 0.53 | 285 |
| 7 | 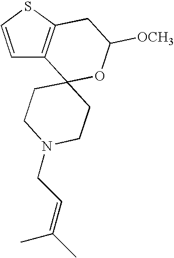 | 0.96 ± 0.25 | 78.1 ± 74.0 |
| 8 | 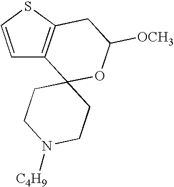 | 4.30 ± 0.44 | 497 ± 294 |
| 9 | 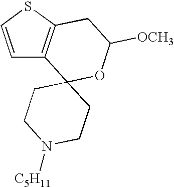 | 1.64 ± 0.53 | 460 |
| 10 | 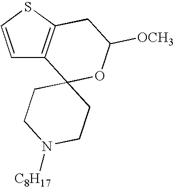 | 4.5 ± 2.0 | 1200 |
| 11 | 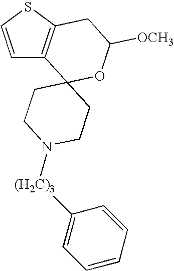 | 2.42 ± 1.66 | 203 |
| 12 | 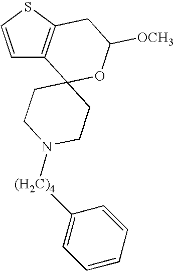 | 7.67 ± 2.15 | 99.2 ± 21.9 |
| 13 | 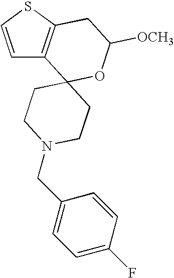 | 3.39 ± 4.54 | 425 |
| 14 | 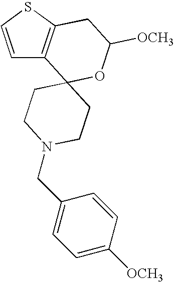 | 2.24 ± 0.99 | 2970 |
| 15 | 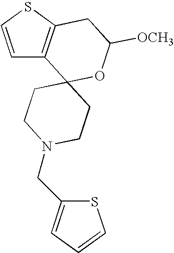 | 2.02 ± 0.61 | 1040 |
| 16 | 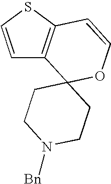 | 1.98 ± 0.89 | 41.9 ± 43.4 |
| 17 | 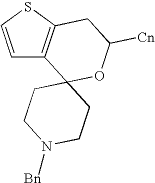 | 1.40 ± 0.32 | 783 |
| 18 | 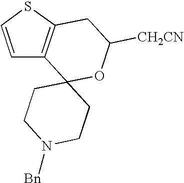 | 3.14 ± 3.33 | 6%* |
| 19 | 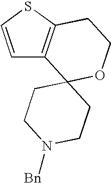 | 0.31 ± 0.11 | 9.6 ± 8.7 |
| 20 | 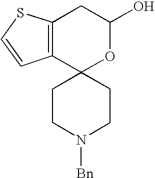 | 3.6 ± 1.16 | 391 |
| 21 | 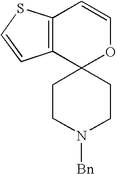 | 1.98 ± 0.89 | 60.8 ± 30.0 |
| 21a | 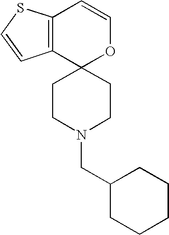 | 1.65 ± 0.15 | 7.32 |
| 22 | 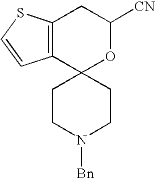 | 1.40 ± 0.32 | 783 |
| 22a | 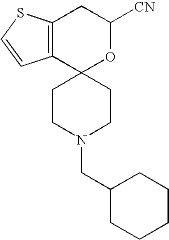 | 0.46 ± 0.07 | 25.8 ± 6.9 |
| 23 | 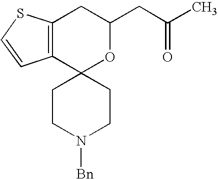 | 9.42 ± 8.0 | 1610 |
| 23a | 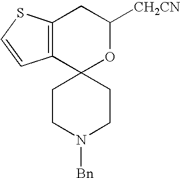 | 3.14 ± 3.33 | 6% |
| 24 | 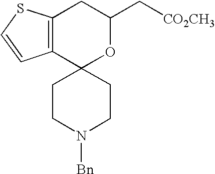 | 12.4 ± 1.2 | 5000 |
| 25 | 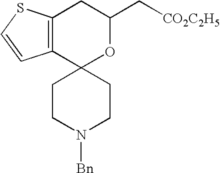 | 20.2 ± 4.9 | 2850 |
| 26 | 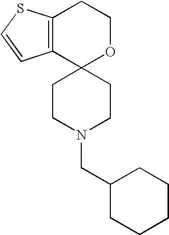 | 0.66 ± 0.27 | 3.27 ± 0.53 |
| 26a | 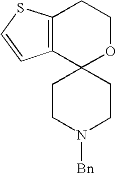 | 0.31 ± 0.11 | 9.6 ± 8.7 |
| 27 | 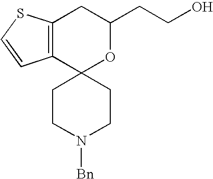 | 27.1 ± 7.5 | 11% |
| 28 | 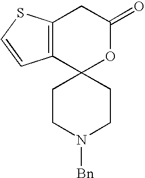 | 296 | 0% |
| 29 | 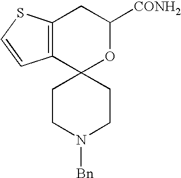 | 350 | 63% |
| 30 | 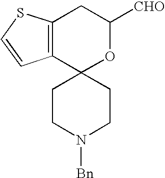 | | |
| 31 | 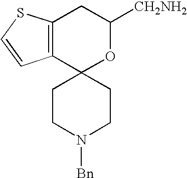 | 98.0 | 7400 |
| 32 | 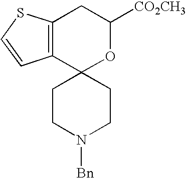 | 29.3 ± 5.5 | 6600 |
| 33 | 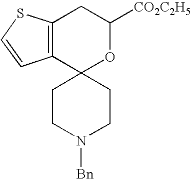 | 144 | 0% |
| 34 | 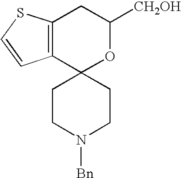 | 5.4 ± 2.55 | 4300 |
| 35 | 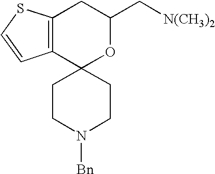 | 62.5 | 8100 |
| 36 | 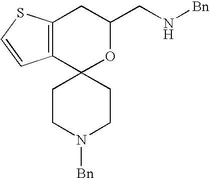 | 196 ± 41.2 | 621 |
| 37 | 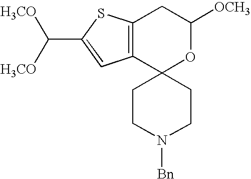 | 2.33 ± 0.65 | 4280 |
| 38 | 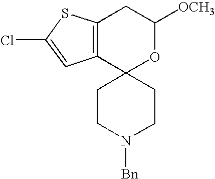 | 1.25 ± 0.11 | 49% |
| 39 | 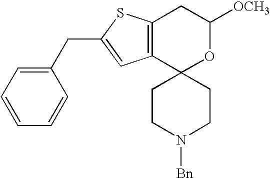 | 3.26 ± 1.15 | 411 |
| 40 | 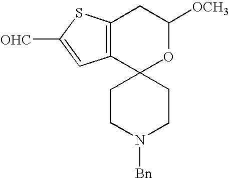 | | |
| 41 | 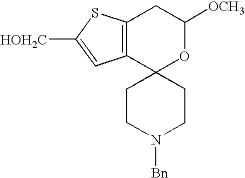 | 5.84 ± 2.03 | 4650 |
| 42 | 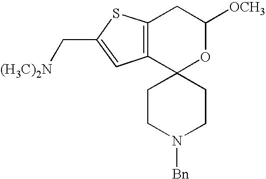 | 16.8 ± 1.10 | 898 |
| 43 | 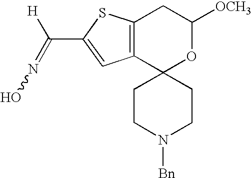 | 38.5 ± 4.6 | 2% |
| 44 | 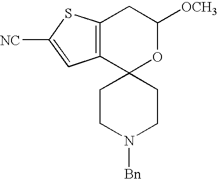 | 1.10 ± 0.15 | 16% |
| 45 | 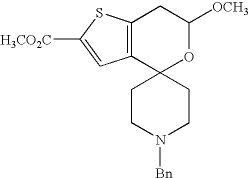 | 10.9 ± 2.2 | 45% |
| 46 | 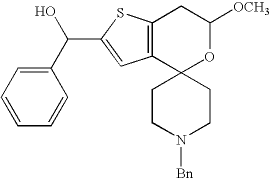 | 3.59 ± 2.51 | 882 ± 17 |
| 47 | 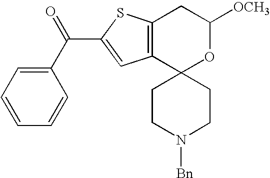 | 3.74 ± 2.37 | 427 ± 163 |

Some other results obtained for Sigma-1 (by version A) are shown in table (II).

TABLE II

| Example | Binding σ1; Ki [nM] |
|---|---|
| 3 | 2.4 ± 0.3 |
| 5 | 2.2 ± 0.3 |
| 6 | 1.3 ± 0.5 |
| 8 | 5.5 ± 1.2 |
| 9 | 2.9 ± 0.5 |
| 13 | 2.3 ± 0.4 |
| 17 | 4.5 ± 0.5 |
| 19 | 2.1 ± 0.4 |

### B) In-Vivo

### Effect on Capsaicin in Development of Mechanical Allodynia

This model uses the von-Frey Filaments and is a model to test the effects or symptoms of neuropathic pain, allodynia etc.

Interest of the model:
The injection of 1 μg of capsaicin to experimental animals produces acute pain followed by hyperalgesia/allodynia
The mechanisms involved in capsaicin-induced acute pain and hyperalgesia are relatively well known (mainly activation of peripheral nociceptors and sensitization of spinal cord neurons, respectively)

The test protocol for all tests with of Frey filaments: After habituation mice were first treated with the test-compound (or solvent in controls). Then 1 μg capsaicin (1% DMSO) is injected into their paw resulting in developing pain in the effected paw. The effected paw is then treated with a mechanical stimulus and the latency time before the paw is withdrawn is measured.

Using this pharmacological test the compounds according to example 22 and example 25 showed a clear antiallodynic effect. They were tested at 16 mg/kg i.p. in 8 animals each and respectively achieved 45.67±9.62% and 44.28±9.49% of pain relief.

1. Compound of general formula (I) or (Ia), 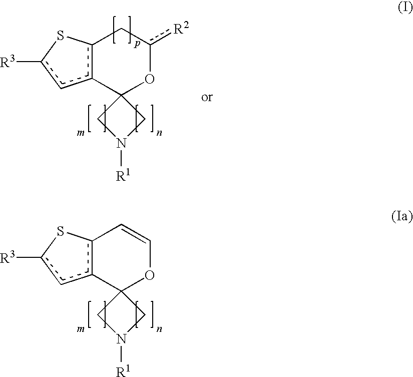
wherein
m is 1, 2 or 3, n is 1, 2 or 3, and m+n is either 3, 4 or 5;
p from 0 or 1; [symbol]
represents either a double or a single bond;
R¹ is hydrogen; an unsubstituted or at least monosubstituted, linear or branched C₁₋₁₈-aliphatic group; an unsubstituted or at least monosubstituted aryl; an unsubstituted or at least monosubstituted heterocyclyl; an unsubstituted or at least monosubstituted cycloalkyl; an unsubstituted or at least monosubstituted alkyl-aryl; an unsubstituted or at least monosubstituted alkyl-heterocyclyl; or an unsubstituted or at least monosubstituted alkyl-cycloalkyl;
R² is hydrogen; an unsubstituted or at least monosubstituted, linear or branched C₁₋₆-aliphatic group; (CH₂)_{q}CN with q being 0, 1, 2, 3 or 4; O—R with R being H or an optionally unsubstituted or at least monosubstituted, linear or branched C₁₋₆-aliphatic group; (CH₂)ₛCOR*; (CH₂)ₛNR*R**; CONR*R**; (CH₂)ₛCO₂R*; or CH═NOR*; (CH₂)ₛOR*;
with
R* being H; or an unsubstituted or at least monosubstituted, linear or branched C₁₋₆-aliphatic group;
R** being an unsubstituted at least monosubstituted, linear or branched C₁₋₆-aliphatic group; or an unsubstituted at least monosubstituted aryl or alkyl-aryl; and
s being 0, 1, or 2;
R³ is hydrogen; halogen; an unsubstituted or at least monosubstituted, linear or branched C₁₋₁₈-aliphatic group; CN; CH(═NOH); NO₂; SO₃H; COR″′; (CH₂)ᵣNR′R″; CH(OR′)(OR″); CO₂R′; an optionally unsubstituted or at least monosubstituted alkyl-aryl with alkyl being unsubstituted or substituted by OH; an unsubstituted or at least monosubstituted alkyl-heterocyclyl with alkyl being unsubstituted or substituted by OH or forming a C(O)-keto-group; or an optionally at least monosubstituted alkyl-cycloalkyl with alkyl being unsubstituted or substituted by OH;
with
R′ being H; or an unsubstituted or at least monosubstituted, linear or branched C₁₋₆-aliphatic group;
R″ being an unsubstituted or at least monosubstituted, linear or branched C₁₋₆-aliphatic group;
R″′ being H; or an unsubstituted or at least monosubstituted, linear or branched C₁₋₆-aliphatic group; or an unsubstituted or at least monosubstituted aryl, an unsubstituted or at least monosubstituted heterocyclyl, or an unsubstituted or at least monosubstituted cycloalkyl; and
r being 0, 1, or 2;
or a stereoisomer, enantiomer or diastereomer, or a racemate thereof or a mixture of at least two stereoisomers, enantiomers and/or diastereomers, in any mixing ratio, or a corresponding salt thereof, or a corresponding solvate thereof;
with the proviso that if R² and R³ are both hydrogen, R¹ may not be methyl.

2. Compound according to claim 1, having a general formula Ic, 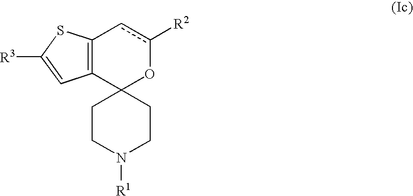
wherein
m is 1, 2 or 3, n is 1, 2 or 3, and m+n is 3, 4 or 5;
p is 0 or 1; [symbol]
represents either a double or a single bond;
R¹ is hydrogen; an unsubstituted or at least monosubstituted, linear or branched C₁₋₁₈-aliphatic group; an unsubstituted or at least monosubstituted aryl; an unsubstituted or at least monosubstituted heterocyclyl; an unsubstituted or at least monosubstituted cycloalkyl; an unsubstituted or at least monosubstituted alkyl-aryl; an unsubstituted or at least monosubstituted alkyl-heterocyclyl; or an unsubstituted or at least monosubstituted alkyl-cycloalkyl;
R² from hydrogen; an unsubstituted or at least monosubstituted, linear or branched C₁₋₆-aliphatic group; (CH₂)_{q}CN with q being 0, 1, 2, 3 or 4; O—R with R being H or an unsubstituted or at least monosubstituted, linear or branched C₁₋₆-aliphatic group; (CH₂)ₛCOR*; (CH₂)ₛNR*R**; CONR*R**; (CH₂)ₛCO₂R*; CH═NOR*; (CH₂)ₛOR*;
with
R* being H; or an unsubstituted or at least monosubstituted, linear or branched C₁₋₆-aliphatic group;
R** being an unsubstituted or at least monosubstituted, linear or branched C₁₋₆-aliphatic group;
or an unsubstituted or at least monosubstituted aryl or alkyl-aryl; and
s being 0, 1, or 2;
R³ is hydrogen; halogen; an unsubstituted or at least monosubstituted, linear or branched C₁₋₁₈-aliphatic group; CN; CH(═NOH); NO₂; SO₃H; COR″′; (CH₂)ᵣNR′R″; CH(OR′)(OR″); CO₂R′; an unsubstituted or at least monosubstituted alkyl-aryl with alkyl being unsubstituted or substituted by OH; an unsubstituted or at least monosubstituted alkyl-heterocyclyl with alkyl being unsubstituted or substituted by OH or forming a C(O)-keto-group; or an unsubstituted or at least monosubstituted alkyl-cycloalkyl with alkyl being unsubstituted or substituted by OH;
with
R′ being H; or an unsubstituted or at least monosubstituted, linear or branched C₁₋₆-aliphatic group;
R″ being an unsubstituted or at least monosubstituted, linear or branched C₁₋₆-aliphatic group;
R″′ being H; an unsubstituted or at least monosubstituted, linear or branched C₁₋₆-aliphatic group;
or an unsubstituted or at least monosubstituted aryl, an unsubstituted or at least monosubstituted heterocyclyl, or an unsubstituted or at least monosubstituted cycloalkyl; and
r being 0, 1, or 2;
or a stereoisomer, enantiomer or diastereomer, a race mate or a mixture of at least two stereoisomers, enantiomers and/or diastereomers, in any mixing ratio, or a corresponding salt thereof, or a corresponding solvate thereof;
with the proviso that if R² and R³ are both hydrogen, R¹ may not be methyl.

3. Compound according to claim 1, characterized in that R¹ is H; an unsubstituted or at least monosubstituted, linear or branched C₁₋₁₈-aliphatic group; an unsubstituted or at least monosubstituted alkyl-aryl; an unsubstituted or at least monosubstituted alkyl-heterocyclyl; or an unsubstituted or at least monosubstituted alkyl-cycloalkyl.

4. Compound according to claim 1, characterized in that R² is hydrogen; an unsubstituted or at least monosubstituted, linear or branched C₁₋₆-aliphatic group; or OR with R being H or an unsubstituted or at least monosubstituted, linear or branched C₁₋₆-aliphatic group; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O) with q being 0 or 1 and R²² being H; or an unsubstituted or at least monosubstituted, linear or branched C₁₋₆-aliphatic group.

5. Compound according to claim 1, characterized in that R³ is hydrogen; halogen; CN; CH(═NOH); an unsubstituted or at least monosubstituted, linear or branched C₁₋₁₀-aliphatic group; CH₂—R³³; C(O)—R³³; C(O)O—R³³; CHOH—R³³; CH₂N(R³³)₂ with R³³ being H; or an unsubstituted or at least monosubstituted, linear or branched C₁₋₆-aliphatic group; or an unsubstituted or at least monosubstituted aryl.

6. Compound according to claim 1, characterized in that each of m and n is independently 1 or 2 and m+n is 3 or 4.

7. Compound according to claim 1, having a general formula Id, 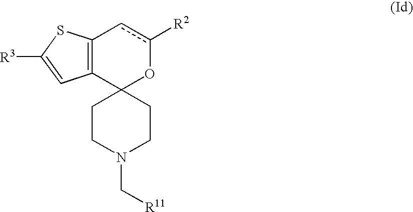
wherein [symbol]
represents either a double or a single bond;
R¹¹ is an unsubstituted or at least monosubstituted, linear or branched C₁₋₉-aliphatic group; an unsubstituted or at least monosubstituted aryl or alkyl-aryl; an unsubstituted or at least monosubstituted heterocyclyl or alkyl-heterocyclyl; an unsubstituted or at least monosubstituted cycloalkyl or alkyl-cycloalkyl;
R² is selected from hydrogen; an unsubstituted or at least monosubstituted, linear or branched C₁₋₆-aliphatic group; or OR with R being H or an unsubstituted or at least monosubstituted, linear or branched C₁₋₆-aliphatic group; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O) with q being 0, or 1 and R²² being H; or an unsubstituted or at least monosubstituted, linear or branched C₁₋₆-aliphatic group;
R³ hydrogen; halogen; CN; CH(═NOH); an unsubstituted at least monosubstituted, linear or branched C₁₋₁₀-aliphatic group; CH₂—R³³; C(O)—R³³; C(O)O—R³³; CHOH—R³³; CH₂N(R³³)₂ with R³³ being H; an unsubstituted or at least monosubstituted, linear or branched C₁₋₆-aliphatic group; or an unsubstituted or at least monosubstituted aryl;
or a stereoisomers, enantiomer or diastereomer, a racemate or a mixture of at least two stereoisomers, enantiomers and/or diastereomers, in any mixing ratio, or a corresponding salt thereof, or a corresponding solvate thereof.

8. Compound according to claim 7, characterized in that R¹¹ is an unsubstituted or at least monosubstituted, linear or branched C₁₋₉-aliphatic group; an unsubstituted or at least monosubstituted aryl or alkyl-aryl; an unsubstituted or at least monosubstituted heterocyclyl; or an unsubstituted or at least monosubstituted cycloalkyl; phenyl, p-methoy-phenyl, cyclohexyl, thiophene, benzyl; branched or linear C₃₋₇-alkyl; or branched or linear C₃₋₇-alkenyl.

9. Compound according to claim 7, characterized in that R² hydrogen; unsubstituted or substituted by at least one of OH, F, or Cl linear or branched C₁₋₆-aliphatic group; or OR with R being H or CH3; (CH₂)_{q}—CN, (CH₂)_{q}—NH-benzyl; (CH₂)_{q}—N(R²²)₂; (CH₂)_{q}—C(O)—N(R²²)₂; (CH₂)_{q}—C(O)—R²² or (CH₂)_{q}—C(O)—O—R²² with q being 0, or 1 and R²² being H, CH₃, or C₂H₅.

10. Compound according to claim 7, characterized in that R³ is hydrogen; halogen; CN; CH(═NOH); a linear or branched, unsubstituted or substituted by at least one of OH, Cl, or F C₁₋₆-aliphatic group; CH₂—R³³; C(O)—R³³; C(O)O—R³³; CHOH—R³³; CH₂N(R³³)₂ with R³³ being H; CH₃; C₂H₅; or phenyl.

11. Compound according to claim 1, characterized in that the compound is selected from the group consisting of:
6′-Methoxy-6′,7′-dihydrospiro[piperidin-4,4′-thieno[3,2-c]pyran;
1-Benzyl-6′-methoxy-6′,7′-dihydrospiro[piperidin-4,4′-thieno[3.2-c]pyran];
6′-Methoxy-1-(2-phenylethyl)-6′,7′-dihydrospiro[piperidin-4,4′-thieno[3.2-c]pyran];
1-(Cyclohexylmethyl)-6′-Methoxy-6′,7′-dihydrospiro[piperidin-4,4′-thieno[3.2-c]pyran];
6′-Methoxy-1-(3-methylbutyl)-6′,7′-dihydrospiro[piperidin-4,4′-thieno[3.2-c]pyran];
6′-Methoxy-1-(3-methylbut-2-enyl)-6′,7′-dihydrospiro[piperidin-4,4′-thieno[3.2-c]pyran];
1-Butyl-6′-methoxy-6′,7′-dihydrospiro[piperidin-4,4′-thieno[3.2-c]pyran];
6′-Methoxy-1-pentyl-6′,7′-dihydrospiro[piperidin-4,4′-thieno[3.2-c]pyran];
6′-Methoxy-1-octyl-6′,7′-dihydrospiro[piperidin-4,4′-thieno[3.2-c]pyran];
3′-Methoxy-1-(3-phenylpropyl)-6′,7′-dihydrospiro[piperidin-4,4′-thieno[3.2-c]pyran];
3′-Methoxy-1-(4-phenylbutyl)-6′,7′-dihydrospiro[piperidin-4,4′-thieno[3.2-c]pyran];
1-(4-Fluorbenzyl)-6′-Methoxy-6′,7′-dihydrospiro[piperidin-4,4′-thieno[3.2-c]pyran];
6′-Methoxy-1-(4-methoxybenzyl)-6′,7′-dihydrospiro[piperidin-4,4′-thieno[3.2-c]pyran];
6′-Methoxy-1-(thien-2-ylmethyl)-6′,7′-dihydrospiro[piperidin-4,4′-thieno[3.2-c]pyran];
1-Benzylspiro[piperidin-4,4′-thieno[3,2-c]pyran];
1-Benzyl-6′,7′-dihydrospiro[piperidin-4,4′-thieno[3,2-c]pyran]-6′-carbonitril;
{1-Benzyl-6′,7′-dihydrospiro[piperidin-4,4′-thieno[3,2-c]pyran]-6′-yl}acetonitril;
1-Benzyl-6′,7′-dihydrospiro[piperidin-4,4′-thieno[3,2-c]pyran];
1-Benzyl-6′,7′-dihydrospiro[piperidin-4,4′-thieno[3,2-c]pyran]-6′-ol
1-(Cyclohexylmethyl)spiro[piperidine-4,4′-thieno[3,2-c]pyrano]
1-(Cyclohexylmethyl)-6′,7′-dihydrospiro[piperidine-4,4′-thieno[3,2-c]pyrano]-6′-carbonitrile
1-(1-Benzyl-6′,7′-dihydrospiro[piperidine-4,4′-thieno[3,2-c]pyrano]-6′-yl)acetone
Methyl-2-(1-benzyl-6′,7′-dihydrospiro[piperidin-4,4′-thieno[3,2-c]pyrano]-6′-yl)-acetate
Ethyl-2-(1-benzyl-6′,7′-dihydrospiro[piperidine-4,4′-thieno[3,2-c]pyrano]-6′-yl)acetate
1-(Cyclohexylmethyl)-6′,7′-dihydrospiro[piperidine-4,4′-thieno[3,2-c]pyrano]
2-(1-Benzyl-6′,7′-dihydrospiro[piperidine-4,4′-thieno[3,2-c]pyrano]-6′-yl)ethanol
1-Benzyl-6′,7′-dihydrospiro[piperidine-4,4′-thieno[3,2-c]pyrano]-6′-carboxamide
1-Benzyl-6′,7′-dihydrospiro[piperidine-4,4′-thieno[3,2-c]pyrano]-6′-carbaldehyde
(1-Benzyl-6′,7′-dihydrospiro[piperidine-4,4′-thieno[3,2-c]pyrano]-6′-yl)methanamine
Methyl-1-benzyl-6′,7′-dihydrospiro[piperidine-4,4′-thieno[3,2-c]pyrano]-6′-carboxylate
Ethyl-1-benzyl-6′,7′-dihydrospiro[piperidine-4,4′-thieno[3,2-c]pyrano]-6′-carboxylate
(1-Benzyl-6′,7′-dihydrospiro[piperidine-4,4′-thieno[3,2-c]pyrano]-6′-yl)methanol
(1-Benzyl-6′,7′-dihydrospiro[piperidine-4,4′-thieno[3,2-c]pyrano]-6′-yl)-N,N-dimethylmethanamine
(1-Benzyl-6′,7′-dihydrospiro[piperidine-4,4′-thieno[3,2-c]pyrano]-6′-yl)-N-benzyl-methanamine
(1-Benzyl-6′-methoxy-6′,7′-dihydrospiro[piperidine-4,4′-thieno[3.2-c]pyrano]-2-carbaldehyde-dimethylacetal
1-Benzyl-2′-chloro-6′-methoxy-6′,7′-dihydrospiro[piperidine-4,4′-thieno[3.2-c]-pyrano]
1,2′-Dibenzyl-6′-methoxy-6′,7′-dihydrospiro[piperidine-4,4′-thieno[3.2-c]pyrano]
(1-Benzyl-6′-methoxy-6′,7′-dihydrospiro[piperidine-4,4′-thieno[3.2-c]pyrano]-2-carbaldehyde
(1-Benzyl-6′-methoxy-6′,7′-dihydrospiro[piperidine-4,4′-thieno[3.2-c]pyrano]-2′-yl)-methanol
(1-Benzyl-6′-methoxy-6′,7′-dihydrospiro[piperidine-4,4′-thieno[3.2-c]pyrano]-2′-yl)-N,N-dimethylmethanamine
(E/Z)-1-Benzyl-6′-methoxy-6′,7′-dihydrospiro[piperidine-4,4′-thieno[3,2-c]pyrano]-2-carbaldehydroxim
1-Benzyl-6′-methoxy-6′,7′-dihydrospiro[piperidine-4,4′-thieno[3,2-c]pyrano]-2-carbonitrile
Methyl-1-benzyl-6′-methoxy-6′,7′-dihydrospiro[piperidine-4,4′-thieno[3,2-c]pyrano]-2-carboxylate
1-(1-Benzyl-6′-methoxy-6′,7′-dihydrospiro[piperidine-4,4′-thieno[3.2-c]pyrano]-2-yl)-1-phenylmethanol; and
1-(1-Benzyl-6′-methoxy-6′,7′-dihydrospiro[piperidine-4,4′-thieno[3.2-c]pyrano]-2-yl)-1-phenylmethanone;
or a stereoisomers, enantiomer or diastereomer, a racemate or a mixture of at least two stereoisomers, enantiomers and/or diastereomers, in any mixing ratio, or a corresponding salt thereof, or a corresponding solvate thereof.

12. Compound according to claim 1, having a general formula Ib, 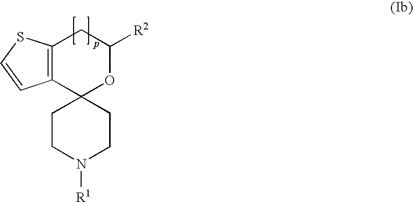
wherein
p is 0 or 1;
R¹ hydrogen; C₁₋₁₀-alkyl, linear or branched; C₂₋₈-alkenyl, linear or branched; unsubstituted or at least mono-substituted C₁₋₆-alkyl-aryl; unsubstituted or at least mono-substituted C₁₋₆-alkyl-heterocyclyl; or unsubstituted or at least mono-substituted C₁₋₆-alkyl-C₄₋₈-cycloalkyl;
R² is H; CN; CH₂CN; OH or a linear or branched OC₁₋₄-alkyl group;
or a stereoisomers, enantiomers or diastereomers, a racemate or a mixture of at least two stereoisomers, enantiomers and/or diastereomers, in any mixing ratio, or a corresponding salt thereof, or a corresponding solvate thereof.

13. Process for the production of a compound of structure 1 of claim 1, wherein a compound of formula (III) 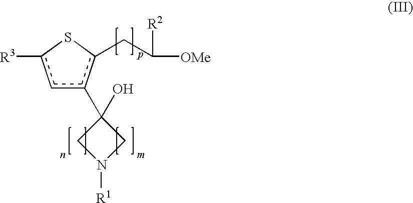
, wherein R¹, R², R³, m, n and p are as defined in claim 1 is reacted with p-toluol sulphonic acid to form the compound

14. Process according to claim 13, wherein
R² is OCH₃; and/or
R³ is H and/or
p is H.

15. Process for the production of a compound according to claim 1, wherein a compound of formula (IIIb) 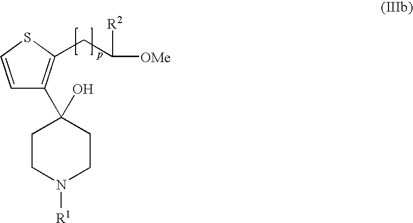
, wherein R¹, R² and p are as defined in claim 1 is reacted with p-toluol sulphonic acid to form the compound.

16. Process according to claim 15, wherein
R² is OCH₃ and/or
p is 1.

17. Process according to claim 12, wherein as a next step a compound according to formula (I) in which R³ is C(O)OR′ is reacted with a alkaline solution in water to form the compound with R¹ being H.

18. A pharmaceutical composition which comprises a compound of claim 1 or a pharmaceutically acceptable salt or solvate thereof, and a pharmaceutically acceptable carrier, adjuvant or vehicle.

19. (canceled)

20. A method for the treatment or prophylaxis of a sigma receptor mediated disease or condition in a subject which comprises administering to the subject a compound of claim 1.

21. A method according to claim 20, wherein the disease is diarrhoea, lipoprotein disorders, metabolic syndrome, treatment of elevated triglyceride levels, chylomicronemia, hyperlipoproteinemia; by perlipidemia, especially mixed hyperlipidemia; hypercholesterolemia, dysbetalipoproteinemia, hypertriglyceridemia including both the sporadic and familial disorder (inherited hypertriglyceridemia), migraine, obesity, arthritis, hypertension, arrhythmia, ulcer, learning, memory and attention deficits, cognition disorders, neurodegenerative diseases, demyelinating diseases, addiction to drugs and chemical substances including cocaine, amphetamine, ethanol and nicotine, tardive diskinesia, ischemic stroke, epilepsy, stroke, depression, stress, psychotic condition, schizophrenia; inflammation, autoimmune diseases or cancer.

22. A method according to claim 20, wherein the disease is pain, especially neuropathic pain, inflammatory pain or other pain conditions, allodynia and/or hyperalgesia, especially mechanical allodynia.

23. A method of claim 20, wherein the disease or condition is anxiety or a disease or condition associated with an undesirable immune response.
